(19) United States
(12) Patent Application Publication
(10) Pub. No.: US 2013/0310276 A1
(43) Pub. Date: Nov. 21, 2013

(54) MICRORNA FOR DIAGNOSIS OF PANCREATIC CANCER

(71) Applicants: Julia Sidenius Johansen, Frederiksberg (DK); Nicolai Aagaard Schultz, Frederiksberg C (DK); Jens Werner, Dossenheim (DE); Morten Wøjdemann, Valby (DK)

(72) Inventors: Julia Sidenius Johansen, Frederiksberg (DK); Nicolai Aagaard Schultz, Frederiksberg C (DK); Jens Werner, Dossenheim (DE); Morten Wøjdemann, Valby (DK)

(73) Assignees: RUPRECHT-KARLS UNIVERSITY OF HEIDELBERG, Heidelberg (DE); HERLEV HOSPITAL, Herlev (DK)

(21) Appl. No.: 13/995,620

(22) PCT Filed: Dec. 21, 2011
(86) PCT No.: PCT/DK11/50509

§ 371 Date: Aug. 6, 2013

Related U.S. Application Data
(60) Provisional application No. 61/425,900, filed on Dec. 22, 2010.

(30) Foreign Application Priority Data
Dec. 22, 2010 (DK) PA 2010 70574

(51) Int. Cl. C12Q 1/68

(52) U.S. Cl. CPC C12Q 1/6886
USPC 506/9; 435/6.14; 435/6.12; 435/6.11; 536/23.1; 536/24.31; 506/16

(57) ABSTRACT

The present invention relates to methods for improving the diagnosis of pancreatic and ampullary adenocarcinomas by making use of specific mi RNA biomarkers and/or mi RNA classifiers.

All patent and non-patent references cited in the application are hereby incorporated by reference in their entirety.

### FIELD OF INVENTION

The present invention relates to a method for improving the diagnosis of pancreatic cancer. MicroRNA (miRNA) biomarkers and classifiers based on a specific miRNA expression pattern are disclosed herein, which distinguishes pancreatic cancer from normal pancreas and/or chronic pancreatitis. This can prove as a valuable diagnostic tool to make possible an early diagnosis of pancreatic cancer thus expediting surgery for individuals with a malignancy of the pancreas in order to reduce the mortality associated therewith.

### BACKGROUND OF INVENTION

Pancreatic cancer (PC) is the 4^{th} most common cause of cancer death in United States and Europe. The prognosis of patients with pancreatic cancer is dismal with a 5-year survival rate of less than 5%.

Early diagnosis of pancreatic cancer is difficult, and most patients therefore have locally advanced or metastatic pancreatic cancer at the time of diagnosis. Thus, novel strategies for early diagnosis of patients with pancreatic cancer are urgently needed.

Diagnosis of pancreatic cancer to date may be performed using one or a combination of the below:
Assessment of symptoms, such as pain in the upper abdomen that typically radiates to the back, loss of appetite and/or nausea and vomiting, significant weight loss, painless jaundice, pale-colored stool, steatorrhea, Trousseau sign, Courvoisier sign, Diabetes mellitus.
Liver function tests
Imaging studies, such as computed tomography (CT scan) and endoscopic ultrasound (EUS)
Endoscopic needle biopsy or surgical excision of the radiologically suspicious tissue.
Cytology
Assessment of risk factors.

MicroRNAs (miRNA or miR) are small, non-coding single-stranded RNA gene products that regulate mRNA translation. The expression of RNA species, such as miRNAs is often deregulated in malignant cells and shows a highly tissue-specific pattern. miRNA biomarkers whose expression is associated with a certain condition, and classifiers based on a mRNA expression profile or signature, may prove to be an ideal diagnostic tool to diagnose pancreatic cancer.

It has been demonstrated that pancreatic cancer has a miRNA expression pattern that differs from normal pancreas and chronic pancreatitis tissue. miRNA profiles therefore offer the potential of improving early diagnosis of pancreas cancer.

In a study by Szafranska et al. (Oncogene 2007; 26:4442-52), miRNA expression alterations were shown to be linked to tumourigenesis and non-neoplastic processes in pancreatic ductal adenocarcinoma. A total of 26 miRNAs were identified, namely miR-205, miR-29c, miR-216, miR-217, miR-375, miR-143, miR-145, miR-146a, miR-148a, miR-196b, miR-93, miR-96, miR-31, miR-210, miR-148b, miR-196a, miR-141, miR-18a, miR-203, miR-150, miR-155, miR-130b, miR-221, miR-222, miR-223 and miR-224. This study used surgical pancreatic resection specimens which were immediately placed on ice, and subsequently snap-frozen and stored at −80° C.

The results by Szafranska et al. have made use of a combination of two miRNAs (miR-196a and miR-217) which has recently been commercialized for diagnosis of pancreas cancer by AsuraGen (see also WO 2008/036765).

WO2008136971, WO2007081680 and WO2008036765 also disclose methods for diagnosing pancreatic cancer by measuring the expression level of at least one miRNA gene product.

A study by Bloomston et al. (JAMA 2007; 297:1901-8) also showed that miRNA expression patterns could differentiate pancreatic adenocarcinoma from normal pancreas and chronic pancreatitis; including miR-93, using micro-dissected pancreas cancer tissue from FFPE tumour blocks.

Further individual miRNAs have been shown to be deregulated in pancreas cancer, such as miR-21 (Dillhoff et al., J Gastrointest Surg 2008; 12:2171-6) and miR-155 (Habbe et al., Cancer Biol Ther 2009; 8:340-6).

While the literature has addressed the miRNA expression pattern of various pancreatic conditions, the present inventors aimed to improve and further develop diagnostic tools for the early diagnosis of pancreatic cancer.

### SUMMARY OF INVENTION

Efforts to make possible an early diagnosis of pancreas cancer are urgently needed, in order to improve the outcome of existing therapies.

The present inventors have further investigated the miRNA expression profile in pancreatic cancer (PC) (comprising pancreatic adenocarcinoma, PAC and ampullary adenocarcinoma, AAC), chronic pancreatitis (CP) and normal pancreas (NP) in order to identify specific miRNAs associated with each condition.

This has lead to the identification of a deregulated subset of miRNAs associated with each of the above-mentioned conditions; including miRNAs which have not previously been identified by others. These miRNAs are potentially useful in diagnosing a condition of the pancreas, such as pancreas cancer.

The present invention thus discloses a sensitive and specific means of separating pancreatic cancer from normal pancreas and/or chronic pancreatitis. The inventors have found that a subset of specific miRNAs are differentially expressed in and associated with each of the above-mentioned conditions, efficiently separating the above-mentioned conditions of the pancreas by employing miRNA classifiers or biomarkers (‘simple combinations’) capable of predicting which of the above categories or classes a certain sample obtained from an individual belongs to.

The present invention is in one aspect directed to the development of a two-way miRNA classifier that distinguishes the combined class of pancreatic carcinoma and ampullary adenocarcinoma from the combined class of normal pancreas and chronic pancreatitis, and comprises or consists of one or more miRNAs selected from the group consisting of miR-198, miR-34c-5p, miR-614, miR-492, miR-10a, miR-622, miR-196b, miR-210, miR-939, miR-649, miR-801, miR-135b*, miR-148a, miR-194*, miR-21, miR-708, miR-222, miR-30a* and miR-323-3p.

The present invention is in another aspect directed to the development of a two-way miRNA classifier that distinguishes the combined class of pancreatic carcinoma and ampullary adenocarcinoma from the combined class of normal pancreas and chronic pancreatitis, and comprises or consists of one or more miRNAs selected from the group consisting of miR-122, miR-135b, miR-135b*, miR-136*, miR-186, miR-196b, miR-198, miR-203, miR-222, miR-23a, miR-34c-5p, miR-451, miR-490-3p, miR-492, miR-509-5p, miR-571, miR-614, miR-622 and miR-939.

The present invention is in another aspect directed to the identification of miRNA biomarkers whose expression level (a) distinguishes between the classes pancreatic carcinoma and normal pancreas, and comprises or consists of miR-411 and/or miR-198; (b) distinguishes the combined class of pancreatic carcinoma and ampullary adenocarcinoma from the combined class of normal pancreas and chronic pancreatitis, and comprises or consists of miR-411 and/or miR-198; (c) distinguishes between the classes pancreatic carcinoma and chronic pancreatitis, and comprises or consists of miR-614 and/or miR-122; (d) distinguishes the combined class of pancreatic carcinoma and ampullary adenocarcinoma from the combined class of normal pancreas and chronic pancreatitis, and comprises or consists of miR-614 and/or miR-122; (e) distinguishes between the classes pancreatic carcinoma and chronic pancreatitis, and comprises or consists of miR-614 and/or miR-93*; (f) distinguishes between the classes pancreatic carcinoma and normal pancreas, and comprises or consists of miR-614 and/or miR-93*; (g) distinguishes the combined class of pancreatic carcinoma and ampullary adenocarcinoma from the combined class of normal pancreas and chronic pancreatitis, and comprises or consists of miR-614 and/or miR-93*; and (h) distinguishes the combined class of pancreatic carcinoma and ampullary adenocarcinoma from the combined class of normal pancreas and chronic pancreatitis, and comprises or consists of two or more of miR-198, miR-34c-5p, miR-614, miR-492, miR-10a, miR-622, miR-196b, miR-210, miR-939, miR-649, miR-801, miR-135b*, miR-148a, miR-194*, miR-21, miR-708, miR-222, miR-30a* and miR-323-3p.

Further potential miRNA biomarkers deregulated in specific conditions of the pancreas are also disclosed herein, which are potentially useful for diagnosis of conditions of the pancreas.

The miRNA classifiers and/or biomarkers may be applied ex vivo to a sample obtained from an individual, in order to facilitate an early and accurate diagnosis of said individual. Said sample may be a tissue sample from the pancreas, or a blood sample, obtained from an individual.

Accordingly, provided herein are methods for diagnosing whether a subject has, or is at risk of developing, pancreatic cancer, comprising the steps of measuring the miRNA expression level in a sample obtained from an individual, and determining whether or not said sample is indicative of the individual of having, or being at risk of developing, pancreatic carcinoma.

The use of the herein disclosed miRNA classifiers and biomarkers can potentially drastically improve the diagnosis of pancreas cancer and allow for an earlier diagnosis, and is as such useful as a stand-alone or an ‘add-on’ method to the existing diagnostic methods currently used for diagnosing pancreas cancer. Early diagnosis of a malignant condition of the pancreas is urgently needed in order to present pancreas cancer patient to surgery at a less advanced stage.

The present invention is also directed to a device comprising probes for at least one miRNA according to the present invention; suitable for measuring the expression level of said at least one miRNA, wherein said device may be used for classifying a sample obtained from an individual and making a diagnosis.

Also provided is a system for performing a diagnosis on an individual, comprising means for analysing the miRNA expression profile of a biological sample, and means for determining if said individual has a condition selected from pancreatic cancer, chronic pancreatitis and normal pancreas.

The present invention is also directed to a computer program product having a computer readable medium, said computer program product providing a system for predicting the diagnosis of an individual, said computer program product comprising means for carrying out any of the steps of any of the methods as disclosed herein.

The results obtained by the present inventors have several advantages. First, the miRNA classifiers identified herein perform better that the commercially available AsuraGen test. This may be partly due to the high number of physical samples included in the present analysis.

Second, for specimens used in the Asuragen test it is recommended to use formalin fixed paraffin embedded tissue (FFPE) containing ≧60% abnormal area content (cancer tissue).

The present invention is based on samples, having the advantage of (a) providing a diagnostic tool which may be used on a sample with a lower cancer tissue content—without e.g. microdissection or otherwise up-concentrating the cancer tissue content; thus omitting a rather complex step of the analysis and allowing diagnosis of a sample obtained by a more straight forward method e.g. a simple biopsy, and (b) including the stroma or desmoplasia of the pancreas in the sample thereby reflecting the actual environment of the tumour and thus not loosing valuable information; which may cause the diagnosis to be more accurate. Furthermore, the present invention may be performed on a sample having a relatively low proportion of tumour cells, such that it may be performed of a fine-needle biopsy.

### DESCRIPTION OF THE DRAWINGS

FIG. 1: Tissue comparison sorted by F-test p-value. Strip charts showing tissue comparison sorted by F-test p-values. Ampullary adenocarcinoma: A-AC; chronic pancreatitis: CH; normal pancreas: NP, Pancreatic cancer: PC.

FIG. 2: Lasso classifier for separating PC and A-AC from normal pancreas and chronic pancreatitis, showing model complexity, sensitivity, positive predictive value and accuracy.

FIG. 3: Combinations of two miRs given as differences between the miRs expressions in the same sample (unnormalized Ct-values). Horizontal lines are showing best cut-off values for separating neoplastic samples from non-neoplastic samples. Colour spots showing tumour % in the tissue samples. The P-values given in 3A, 3C and 3D are for differences in miR expression in PC and chronic pancreatitis. The p-value in 3B is for the differences in miR expression differences in PC and A-AC compared to normal pancreas and chronic pancreatitis.

FIG. 4: Venn-diagram showing overlap of miRs expressed in at least 90% of each class' samples.

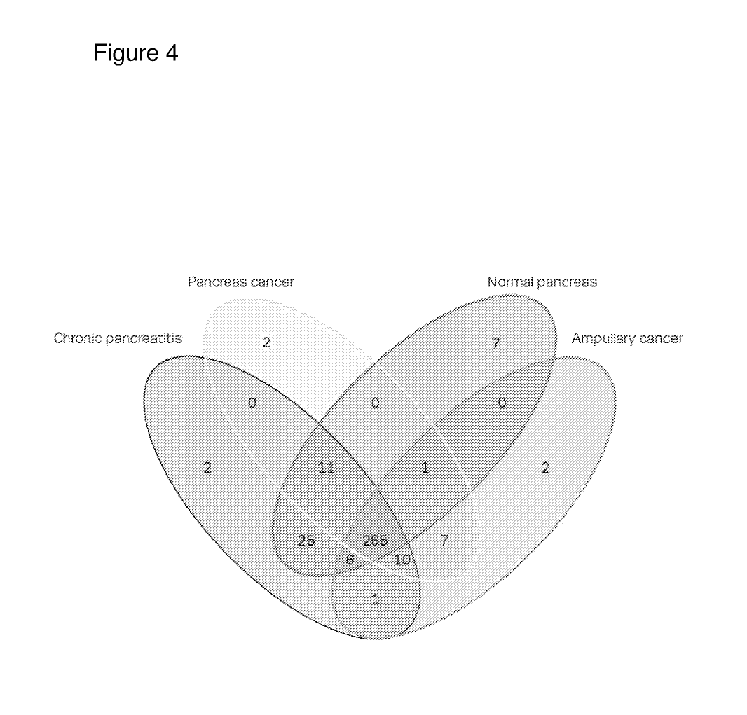

FIG. 5: Hierarchial cluster analysis. PC: green; A-AC: orange; normal pancreas: purple; chronic pancreatitis: pink.

FIG. 6: Heat map of sample clustering for our 19 miR-classifier.

FIG. 7: Scatter plots comparing each tissue sample mean to another tissue sample mean.

FIG. 8: Tissue comparison sorted by ‘normal vs. cancer’ p-value. Tissue comparison density plots for selected miRs.

### DEFINITIONS

Statistical classification is a procedure in which individual items are placed into groups based on quantitative information on one or more characteristics inherent in the items (referred to as traits, variables, characters, etc) and based on a training set of previously labeled items.

A classifier is a prediction model which may distinguish between or characterize samples by classifying a given sample into a predetermined class based on certain characteristics of said sample. A two-way classifier classifies a given sample into one of two predetermined classes, and a three-way classifier classifies a given sample into one of three predetermined classes.

The terms distinction, differentiation, separation, classification and characterisation of a sample are used herein as being capable of predicting with a relatively high sensitivity and specificity if a given sample of unknown diagnosis belongs to the class of pancreas cancer, chronic pancreatitis and/or normal pancreas. The output may be given as a probability of belonging to either class of between 0-1 (for classifiers), or may be estimated directly based on differences in expression levels (for biomarkers).

A ‘biomarker’ may be defined as a biological molecule found in blood, other body fluids, or tissues that is an indicator of a normal or abnormal process, or of a condition or disease. A biomarker may be used to foresee how well the body responds to a treatment for a disease or condition, or may be used to associate a certain disease or condition to a certain value of said biomarker found in e.g. a tissue sample. Biomarkers are also called molecular marker and signature molecule.

‘Collection media’ as used herein denotes any solution suitable for collecting, storing or extracting of a sample for immediate or later retrieval of RNA from said sample.

‘Deregulated’ means that the expression of a gene or a gene product is altered from its normal baseline levels; comprising both up- and down-regulated.

The term “Individual” refers to vertebrates, particular members of the mammalian species, preferably primates including humans. As used herein, ‘subject’ and ‘individual’ may be used interchangeably.

The term “Kit of parts” as used herein provides a device for measuring the expression level of at least one miRNA as identified herein, and at least one additional component. The additional component may be used simultaneously, sequentially or separately with the device. The additional component may in one embodiment be means for extracting RNA, such as miRNA, from a sample; reagents for performing microarray analysis, reagents for performing QPCR analysis and/or instructions for use of the device and/or additional components.

The term “natural nucleotide” or “nucleotide” refers to any of the four deoxyribonucleotides, dA, dG, dT, and dC (constituents of DNA), and the four ribonucleotides, A, G, U, and C (constituents of RNA). Each natural nucleotide comprises or essentially consists of a sugar moiety (ribose or deoxyribose), a phosphate moiety, and a natural/standard base moiety. Natural nucleotides bind to complementary nucleotides according to well-known rules of base pairing (Watson and Crick), where adenine (A) pairs with thymine (T) or uracil (U); and where guanine (G) pairs with cytosine (C), wherein corresponding base-pairs are part of complementary, anti-parallel nucleotide strands. The base pairing results in a specific hybridization between predetermined and complementary nucleotides. The base pairing is the basis by which enzymes are able to catalyze the synthesis of an oligonucleotide complementary to the template oligonucleotide. In this synthesis, building blocks (normally the triphosphates of ribo or deoxyribo derivatives of A, T, U, C, or G) are directed by a template oligonucleotide to form a complementary oligonucleotide with the correct, complementary sequence. The recognition of an oligonucleotide sequence by its complementary sequence is mediated by corresponding and interacting bases forming base pairs. In nature, the specific interactions leading to base pairing are governed by the size of the bases and the pattern of hydrogen bond donors and acceptors of the bases. A large purine base (A or G) pairs with a small pyrimidine base (T, U or C). Additionally, base pair recognition between bases is influenced by hydrogen bonds formed between the bases. In the geometry of the Watson-Crick base pair, a six membered ring (a pyrimidine in natural oligonucleotides) is juxtaposed to a ring system composed of a fused, six membered ring and a five membered ring (a purine in natural oligonucleotides), with a middle hydrogen bond linking two ring atoms, and hydrogen bonds on either side joining functional groups appended to each of the rings, with donor groups paired with acceptor groups.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR), and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g. alpha-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes e.g. so-called “peptide nucleic acids,” which comprise naturally-occurring or modified nucleic acid bases attached to a polyamide backbone. Nucleic acids can be either single stranded or double stranded. In an aspect of the present invention, ‘nucleic acid’ is meant to comprise antisense oligonucleotides (ASO), small inhibitory RNAs (sRNA), short hairpin RNA (shRNA) and microRNA (miRNA).

A “polypeptide” or “protein” is a polymer of amino acid residues preferably joined exclusively by peptide bonds, whether produced naturally or synthetically. The term “polypeptide” as used herein covers proteins, peptides and polypeptides, wherein said proteins, peptides or polypeptides may or may not have been post-translationally modified. Post-translational modification may for example be phosphorylation, methylation and glycosylation.

A ‘probe’ as used herein refers to a hybridization probe. A hybridization probe is a (single-stranded) fragment of DNA or RNA of variable length (usually 100-1000 bases long), which is used in DNA or RNA samples to detect the presence of nucleotide sequences (the DNA target) that are complementary to the sequence in the probe. The probe thereby hybridizes to single-stranded nucleic acid (DNA or RNA) whose base sequence allows probe-target base pairing due to complementarity between the probe and target. To detect hybridization of the probe to its target sequence, the probe is tagged (or labelled) with a molecular marker of either radioactive or fluorescent molecules. DNA sequences or RNA transcripts that have moderate to high sequence similarity to the probe are then detected by visualizing the hybridized probe. Hybridization probes used in DNA microarrays refer to DNA covalently attached to an inert surface, such as coated glass slides or gene chips, and to which a mobile cDNA target is hybridized.

Due to the imprecision of standard analytical methods, molecular weights and lengths of polymers are understood to be approximate values. When such a value is expressed as “about” X or “approximately” X, the stated value of X will be understood to be accurate to +/−20%, such as +/−10%, for example +/−5%.

### DETAILED DESCRIPTION OF THE INVENTION

### The Pancreas

The pancreas is a gland organ in the digestive and endocrine system of vertebrates. It is both an endocrine gland producing several important hormones, including insulin, glucagon, and somatostatin, as well as an exocrine gland, secreting pancreatic juice containing digestive enzymes that pass to the small intestine. These enzymes help to further break down the carbohydrates, proteins, and fats in the chyme.

Microscopically, stained sections of the pancreas reveal two different types of parenchymal tissue. Lightly staining clusters of cells are called islets of Langerhans, which produce hormones that underlie the endocrine functions of the pancreas. Darker staining cells form acini connected to ducts. Acinar cells belong to the exocrine pancreas and secrete digestive enzymes into the gut via a system of ducts.

Four main cell types exist in the islets of Langerhans that can be classified by their secretion: α (alpha) cells secrete glucagon (increase glucose in blood), β (beta) cells secrete insulin (decrease glucose in blood), δ (delta) cells secrete somatostatin (regulates α and β cells), and PP cells secrete pancreatic polypeptide.

The pancreas receives regulatory innervation via hormones in the blood and through the autonomic nervous system. These two inputs regulate the secretory activity of the pancreas.

The pancreas lies in the epigastrium and left hypochondrium areas of the abdomen. The head lies within the concavity of the duodenum. The uncinate process emerges from the lower part of head, and lies deep to superior mesenteric vessels. The neck is the constricted part between the head and the body. The body lies behind the stomach. The tail is the left end of the pancreas. It lies in contact with the spleen and runs in the lienorenal ligament.

### Pancreatic Cancer

Neoplasia or cancer is the abnormal proliferation of cells, resulting in a structure known as a neoplasm. The growth of this clone of cells exceeds, and is uncoordinated with, that of the normal tissues around it. It usually causes a lump or tumour. Neoplasias may be benign (adenoma) or malignant (carcinoma).

Pancreatic or pancreas neoplasia, pancreatic or pancreas cancer (PC), pancreatic or pancreas carcinoma may be used interchangeably throughout the present application. Normal pancreas is abbreviated NP.

Pancreatic cancer is a malignant neoplasm of the pancreas. Patients diagnosed with pancreatic cancer have a poor prognosis, partly because the cancer usually causes no symptoms early on, leading to locally advanced or metastatic disease at the time of diagnosis. Median survival from diagnosis is around 3 to 6 months; 5-year survival is less than 5%. Pancreatic cancer has one of the highest fatality rates of all cancers, and is the fourth-highest cancer killer in the US and Europe.

The vast majority; about 95% of exocrine pancreatic cancers are pancreatic adenocarcinomas; PAC (also known as pancreatic ductal adenocarcinoma, PDAC). Accordingly, PC and PAC are often used as synonyms. The remaining 5% include adenosquamous carcinomas, signet ring cell carcinomas, hepatoid carcinomas, colloid carcinomas, undifferentiated carcinomas, and undifferentiated carcinomas with osteoclast-like giant cells. Exocrine pancreatic tumours are far more common than pancreatic endocrine tumours, which make up about 1% of total cases.

Desmoplasia is the growth of fibrous or connective tissue. It is also called desmoplastic reaction to emphasize that it is secondary to a neoplasm, causing dense fibrosis around the tumour. Desmoplasia is usually only associated with malignant neoplasms, such as plancreas cancer which can evoke a fibrosis response by invading healthy tissue.

Treatment of pancreatic cancer depends on the stage of the cancer. The Whipple procedure is the most common surgical treatment for cancers involving the head of the pancreas. This procedure involves removing the pancreatic head and the curve of the duodenum together (pancreato-duodenectomy), making a bypass for food from stomach to jejunum (gastro-jejunostomy) and attaching a loop of jejunum to the cystic duct to drain bile (cholecysto-jejunostomy). It can be performed only if the patient is likely to survive major surgery and if the cancer is localized without invading local structures or metastasizing. It can, therefore, be performed in only the minority of cases.

Cancers of the tail of the pancreas can be resected using a procedure known as a distal pancreatectomy. Recently, localized cancers of the pancreas have been resected using minimally invasive (laparoscopic) approaches.

Surgery can be performed for palliation, if the malignancy is invading or compressing the duodenum or colon. In that case, bypass surgery might overcome the obstruction and improve quality of life, but it is not intended as a cure.

After surgery, adjuvant chemotherapy has been shown to significantly increase the 5-year survival, and should be offered if the patient is fit after surgery. Addition of radiation therapy is a hotly debated topic, due to the lack of any large randomized studies to show any survival benefit of this strategy.

In patients not suitable for resection with curative intent, palliative chemotherapy may be used to improve quality of life and gain a modest survival benefit.

### Ampullary Adenocarcinoma

Ampullary adenocarcinomas (A-AC or AAC); also known as adenocarcinoma of the Ampulla of Vater, is a malignant tumour arising in the last centimeter of the common bile duct, where it passes through the wall of the duodenum and ampullary papilla. The pancreatic duct (of Wirsung) and common bile duct merge and exit by way of the ampulla into the duodenum. The ductal epithelium in these areas is columnar and resembles that of the lower common bile duct.

AAC is relatively uncommon, accounting for approximately 0.2% of gastrointestinal tract malignancies and approximately 7% of all periampullary carcinomas

The prognosis of AAC is better than for PAC with a 5-years survival after surgery of 40%. One of the reasons is that even small A-AC cause jaundice so more patients are operated at an early tumour stage and without lymph node metastasis.

### Pancreatitis

Chronic pancreatitis (CP) is commonly defined as a continuing, chronic inflammatory process of the pancreas, characterized by irreversible morphological changes. This chronic inflammation can lead to chronic abdominal pain and/or impairment of endocrine and exocrine function of the pancreas. Chronic pancreatitis usually is envisioned as an atrophic fibrotic gland with dilated ducts and calcifications. However, findings on conventional diagnostic studies may be normal in the early stages of chronic pancreatitis, as the inflammatory changes can be seen only by histologic examination.

By definition, chronic pancreatitis is a completely different process from acute pancreatitis. In acute pancreatitis, the patient presents with acute and severe abdominal pain, nausea, and vomiting. The pancreas is acutely inflamed (neutrophils and oedema), and the serum levels of pancreatic enzymes (amylase and lipase) are elevated. Full recovery is observed in most patients with acute pancreatitis, whereas in chronic pancreatitis, the primary process is a chronic, irreversible inflammation (monocyte and lymphocyte) that leads to fibrosis with calcification. The patient with chronic pancreatitis clinically presents with chronic abdominal pain and normal or mildly elevated pancreatic enzyme levels; when the pancreas loses its endocrine and exocrine function, the patient presents with diabetes mellitus and steatorrhea.

### Diagnosing Pancreatic Cancer at Present

Pancreatic cancer is sometimes called a “silent killer” because early pancreatic cancer often does not cause symptoms, and the later symptoms are usually nonspecific and varied. Therefore, pancreatic cancer is often not diagnosed until it is advanced.

The clinical and histological similarity between pancreatic cancer and chronic pancreatitis adds another dimension to the diagnostic challenge.

Common symptoms of PC include:
Pain in the upper abdomen that typically radiates to the back (seen in carcinoma of the body or tail of the pancreas)
Loss of appetite and/or nausea and vomiting
Significant weight loss
Painless jaundice (yellow skin/eyes, dark urine) when a cancer of the head of the pancreas (about 60% of cases) obstructs the common bile duct as it runs through the pancreas. This may also cause pale-colored stool and steatorrhea.
Trousseau sign, in which blood clots form spontaneously in the portal blood vessels, the deep veins of the extremities, or the superficial veins anywhere on the body, is sometimes associated with pancreatic cancer.
Diabetes mellitus, or elevated blood sugar levels. Many patients with pancreatic cancer develop diabetes months to even years before they are diagnosed with pancreatic cancer, suggesting new onset diabetes in an elderly individual may be an early warning sign of pancreatic cancer.

The initial presentation varies according to location of the cancer. Malignancies in the pancreatic body or tail usually present with pain and weight loss, while those in the head of the gland typically present with steatorrhea, weight loss, and jaundice. The recent onset of atypical diabetes mellitus, a history of recent but unexplained thrombophlebitis (Trousseau sign), or a previous attack of pancreatitis are sometimes noted. Courvoisier sign defines the presence of jaundice and a painlessly distended gallbladder as strongly indicative of pancreatic cancer, and may be used to distinguish pancreatic cancer from gallstones. Tiredness, irritability and difficulty eating because of pain also exist. Pancreatic cancer is often discovered during the course of the evaluation of aforementioned symptoms.

Liver function tests can show a combination of results indicative of bile duct obstruction (raised conjugated bilirubin, γ-glutamyl transpeptidase and alkaline phosphatase levels).

Imaging studies, such as computed tomography (CT scan) and endoscopic ultrasound (EUS) can be used to identify the location and form of the cancer.

An assessment of risk factors may also help make a diagnosis, comprising the occurrence of pancreatic cancer in the family, age above 60 years, male gender, smoking, obesity, diabetes mellitus, chronic pancreatitis, Helicobacter pylori infection, gingivitis or periodontal disease, diets low in vegetables and fruits, high in red meat, and/or high in sugar-sweetened drinks.

A definitive diagnosis is made by an endoscopic needle biopsy or surgical excision of the radiologically suspicious tissue. Endoscopic ultrasound is often used to visually guide the needle biopsy procedure.

The most common form of pancreatic cancer (ductal adenocarcinoma) is typically characterized by moderately to poorly differentiated glandular structures on microscopic examination. Pancreatic cancer has an immunohistochemical profile that is similar to hepatobiliary cancers (e.g. cholangiocarcinoma) and some stomach cancers; thus, it may not always be possible to be certain that a tumour found in the pancreas arose from it.

CA 19-9 (carbohydrate antigen 19.9) is a tumour marker or biomarker that is frequently elevated in pancreatic cancer (detectable in the serum). It is used mainly for monitoring and early detection of recurrence after treatment of patients with known PC. However, it lacks sensitivity and specificity. CA 19-9 might be normal early in the course, and could also be elevated because of benign causes of biliary obstruction. Further 10% of patients with PC are unable to produce CA 19-9.

Thus, novel strategies for early diagnosis of patients with pancreatic cancer are urgently needed. The use of miRNA expression levels as biomarkers in blood samples or tissue samples is an emerging research field aimed to improve the diagnostic tools for pancreas cancer.

The methods disclosed herein provide a tool for improving the early diagnosis of pancreas cancer, thus improving prognosis of affected individuals.

The miRNA classifiers and/or biomarkers as disclosed herein may in one embodiment be used in the clinic alone (stand alone diagnostic); i.e. without employing further diagnostic methods.

In another embodiment, the miRNA classifiers and/or biomarkers as disclosed herein may be used in the clinic as an add-on or supplementary diagnostic tool or method, which improves the diagnosis of pancreas cancer by combining the output of said miRNA classifier and/or biomarker level with the output of one or more of the above-mentioned conventional diagnostic techniques to improve the accuracy of said diagnosis of pancreas cancer.

### Nucleic Acids

A nucleic acid is a biopolymeric macromolecule composed of chains of monomeric nucleotides. In biochemistry these molecules carry genetic information or form structures within cells. The most common nucleic acids are deoxyribonucleic acid (DNA) and ribonucleic acid (RNA). Each nucleotide consists of three components: a nitrogenous heterocyclic base (the nucleobase component), which is either a purine or a pyrimidine; a pentose sugar (backbone residues); and a phosphate group (internucleoside linkers). A nucleoside consists of a nucleobase (often simply referred to as a base) and a sugar residue in the absence of a phosphate linker. Nucleic acid types differ in the structure of the sugar in their nucleotides—DNA contains 2-deoxyriboses while RNA contains ribose (where the only difference is the presence of a hydroxyl group). Also, the nitrogenous bases found in the two nucleic acid types are different: adenine, cytosine, and guanine are found in both RNA and DNA, while thymine only occurs in DNA and uracil only occurs in RNA. Other rare nucleic acid bases can occur, for example inosine in strands of mature transfer RNA. Nucleobases are complementary, and when forming base pairs, must always join accordingly: cytosine-guanine, adenine-thymine (adenine-uracil when RNA). The strength of the interaction between cytosine and guanine is stronger than between adenine and thymine because the former pair has three hydrogen bonds joining them while the latter pair has only two. Thus, the higher the GC content of double-stranded DNA, the more stable the molecule and the higher the melting temperature.

Nucleic acids are usually either single-stranded or double-stranded, though structures with three or more strands can form. A double-stranded nucleic acid consists of two single-stranded nucleic acids held together by hydrogen bonds, such as in the DNA double helix. In contrast, RNA is usually single-stranded, but any given strand may fold back upon itself to form secondary structure as in tRNA and rRNA.

The sugars and phosphates in nucleic acids are connected to each other in an alternating chain, linked by shared oxygens, forming a phosphodiester bond. In conventional nomenclature, the carbons to which the phosphate groups attach are the 3′ end and the 5′ end carbons of the sugar. This gives nucleic acids polarity. The bases extend from a glycosidic linkage to the 1′ carbon of the pentose sugar ring. Bases are joined through N-1 of pyrimidines and N-9 of purines to 1′ carbon of ribose through N-13 glycosyl bond.

microRNA

MicroRNAs (miRNA) are single-stranded RNA molecules of about 19-25 nucleotides in length, which regulate gene expression. miRNAs are either expressed from non-protein-coding transcripts or mostly expressed from protein coding transcripts. They are processed from primary transcripts known as pri-miRNA to shorter stem-loop structures called pre-miRNA and finally to functional mature miRNA. Mature miRNA molecules are partially complementary to one or more messenger RNA (mRNA) molecules, and their main function is to inhibit gene expression. This may occur by preventing mRNA translation or increasing mRNA turnover/degradation.

The transcripts encoding miRNAs are much longer than the processed mature miRNA molecule; miRNAs are first transcribed as primary transcripts or pri-miRNA with a cap and poly-A tail by RNA polymerase II and processed to short, 70-nucleotide stem-loop structures known as pre-miRNA in the cell nucleus. This processing is performed in animals (including humans) by a protein complex known as the Microprocessor complex, consisting of the ribonuclease III Drosha and the double-stranded RNA binding protein Pasha. These pre-miRNAs are then exported to the cytoplasm by Exportin-5/Ran-GTP and processed to mature miRNAs by interaction with the ribonuclease III Dicer and separation of the miRNA duplexes. The mature single-stranded miRNA is incorporated into a RNA-induced silencing complex (RISC)-like ribonucleoprotein particle (miRNP). The RISC complex is responsible for the gene silencing observed due to miRNA expression and RNA interference. The pathway is different for miRNAs derived from intronic stem-loops; these are processed by Dicer but not by Drosha.

When Dicer cleaves the pre-miRNA stem-loop, two complementary short RNA molecules are formed, but only one is integrated into the RISC complex. This strand is known as the guide strand and is selected by the argonaute protein, the catalytically active RNase in the RISC complex, on the basis of the stability of the 5′ end. The remaining strand, known as the anti-guide or passenger strand, is degraded as a RISC complex substrate. After integration into the active RISC complex, miRNAs base pair with their complementary mRNA molecules. This may induce mRNA degradation by argonaute proteins, the catalytically active members of the RISC complex, or it may inhibit mRNA translation into proteins without mRNA degradation.

The function of miRNAs appears to be mainly in gene regulation. For that purpose, an miRNA is (partly) complementary to a part of one or more mRNAs. Animal (including human) miRNAs are usually complementary to a site in the 3′ UTR. The annealing of the miRNA to the mRNA then inhibits protein translation, and sometimes facilitates cleavage of the mRNA (depending on the degree of complementarity). In such cases, the formation of the double-stranded RNA through the binding of the miRNA to mRNA inhibits the mRNA transcript through a process similar to RNA interference (RNAi). Further, miRNAs may regulate gene expression post-transcriptionally at the level of translational inhibition at P-bodies. These are regions within the cytoplasm consisting of many enzymes involved in mRNA turnover; P bodies are likely the site of miRNA action, as miRNA-targeted mRNAs are recruited to P bodies and degraded or sequestered from the translational machinery. In other cases it is believed that the miRNA complex blocks the protein translation machinery or otherwise prevents protein translation without causing the mRNA to be degraded. miRNAs may also target methylation of genomic sites which correspond to targeted mRNAs. miRNAs function in association with a complement of proteins collectively termed the miRNP (miRNA ribonucleoprotein complex).

Under a standard nomenclature system, miRNA names are assigned to experimentally confirmed miRNAs before publication of their discovery. The prefix “mir” is followed by a dash and a number, the latter often indicating order of naming. For example, mir-123 was named and likely discovered prior to mir-456. The uncapitalized “mir-” refers to the pre-miRNA, while a capitalized “miR-” refers to the mature form. miRNAs with nearly identical sequences bar one or two nucleotides are annotated with an additional lower case letter. For example, miR-123a would be closely related to miR-123b. miRNAs that are 100% identical but are encoded at different places in the genome are indicated with additional dash-number suffix: miR-123-1 and miR-123-2 are identical but are produced from different pre-miRNAs. Species of origin is designated with a three-letter prefix, e.g., hsa-miR-123 would be from human (Homo sapiens) and oar-miR-123 would be a sheep (Ovis aries) miRNA. Other common prefixes include ‘v’ for viral (miRNA encoded by a viral genome) and ‘d’ for Drosophila miRNA. microRNAs originating from the 3′ or 5′ end of a pre-miRNA are denoted with a −3p or -5p suffix. (In the past, this distinction was also made with ‘s’ (sense) and ‘as’ (antisense)). An asterisk following the name indicates that the miRNA is an anti-miRNA to the miRNA without an asterisk (e.g. miR-123* is an anti-miRNA to miR-123). When relative expression levels are known, an asterisk following the name indicates a miRNA expressed at low levels relative to the miRNA in the opposite arm of a hairpin. For example, miR-123 and miR-123* would share a pre-miRNA hairpin, but relatively more miR-123 would be found in the cell.

As used herein, it is understood that ‘miR-’ and ‘hsa-miR’ is used interchangeably; the results of the present invention are obtained from human samples and human miRNAs are examined.

miRBase is the central online repository for microRNA (miRNA) nomenclature, sequence data, annotation and target prediction, and may be accessed via http://www.mirbase.org/. The miRNA names used herein throughout can be accessed via this link, and specifics retrieved. See also Griffiths-Jones et al, “miRBase: tools for microRNA genomics”, Nucleic Acids Research, 2008, Vol. 36, Database issue D154-D158.

### Biomarker

A biomarker, or biological marker, is in general a substance used as an indicator of a biological state. It is a characteristic that is objectively measured and evaluated as an indicator of normal biological processes, pathogenic processes, or pharmacologic responses to a therapeutic intervention.

More specifically, a biomarker indicates a change in expression or state of a protein or miRNA that correlates with the risk or progression of a disease, or with the susceptibility of the disease to a given treatment.

A biomarker, such as a miRNA biomarker, may be correlated to a certain condition based on differences in miRNA expression levels between a sample and a control. If a certain miRNA biomarker is found to be deregulated in a sample as compared to a (normal) control level, the sample has a certain probability of being associated with a certain condition.

According to the present invention, the miRNA biomarkers identified herein are able to correlate a deregulated expression level of said miRNA to a diagnosis selected from pancreas cancer (such as PAC and/or AAC), chronic pancreatitis or normal pancreas.

It follows that the expression of one biomarker may in itself be deregulated in a condition (e.g. cancer) as compared to another condition (e.g. control); or it may be the relationship between the expression levels of two or more biomarkers that is telling of a particular condition; i.e. the relative difference in expression levels between two biomarkers.

miRNA Biomarkers of the Present Invention

The present invention is in one aspect directed to the identification of miRNA biomarkers that may be used to
a) distinguish between the classes pancreatic carcinoma and normal pancreas, and comprises or consists of miR-411 and/or miR-198; or
b) distinguish the combined class of pancreatic carcinoma and ampullary adenocarcinoma from the combined class of normal pancreas and chronic pancreatitis, and comprises or consists of miR-411 and/or miR-198; or
c) distinguish between the classes pancreatic carcinoma and chronic pancreatitis, and comprises or consists of miR-614 and/or miR-122; or
d) distinguish the combined class of pancreatic carcinoma and ampullary adenocarcinoma from the combined class of normal pancreas and chronic pancreatitis, and comprises or consists of miR-614 and/or miR-122; or
e) distinguish between the classes pancreatic carcinoma and chronic pancreatitis, and comprises or consists of miR-614 and/or miR-93*; or
f) distinguish between the classes pancreatic carcinoma and normal pancreas, and comprises or consists of miR-614 and/or miR-93*; or
g) distinguish the combined class of pancreatic carcinoma and ampullary adenocarcinoma from the combined class of normal pancreas and chronic pancreatitis, and comprises or consists of miR-614 and/or miR-93*; or
h) distinguish the combined class of pancreatic carcinoma and ampullary adenocarcinoma from the combined class of normal pancreas and chronic pancreatitis, and comprises or consists of two or more of miR-198, miR-34c-5p, miR-614, miR-492, miR-10a, miR-622, miR-196b, miR-210, miR-939, miR-649, miR-801, miR-135b*, miR-148a, miR-194*, miR-21, miR-708, miR-222, miR-30a* and miR-323-3p.

It is contemplated that the expression level of at least one of said miRNAs in one embodiment is measured in a sample from an individual, and said miRNA expression level as compared to a control or baseline level is then associated with a specific condition.

In a particular embodiment, the difference between the expression levels of two miRNAs is calculated; wherein said difference in expression levels between said two miRNAs may be used to correlate said difference in miRNA expression level to a certain condition of the pancreas. Said difference may thus be a relative difference.

In one embodiment, said biomarkers are used in combination (‘simple combination’); i.e. the expression level of at least the two miRNAs according to a) to g) immediately herein above are both used in combination to distinguish or separate the potential conditions of the pancreas.

In one embodiment, the combination of miR-411 and miR-198 is used to separate PC from NP. This specific combination is shown herein to separate PC from NP with a p-value of 5.17e-43.

In another embodiment, the combination of miR-411 and miR-198 is used to separate the combined group of PC and AAC from the combined group of NP and CP. This specific combination is shown herein to separate PC/AAC from NP/CP with a p-value of 4.64e-49.

In one embodiment, the combination of miR-614 and miR-122 is used to separate PC from CP. This specific combination is shown herein to separate PC from CP with a p-value of 7.76e-18.

In another embodiment, the combination of miR-614 and miR-122 is used to separate the combined group of PC and AAC from the combined group of NP and CP. This specific combination is shown herein to separate PC/AAC from NP/CP with a p-value of 8.64e-38.

In one embodiment, the combination of miR-614 and miR-93* is used to separate PC from CP. This specific combination is shown herein to separate PC from CP with a p-value of 9.01e-18.

In another embodiment, the combination of miR-614 and miR-93* is used to separate PC from NP. This specific combination is shown herein to separate PC from NP with a p-value of 5.56e-24.

In yet another embodiment, the combination of miR-614 and miR-93* is used to separate the combined group of PC and AAC from the combined group of NP and CP. This specific combination is shown herein to separate PC/AAC from NP/CP with a p-value of 2.64e-42.

In a particular embodiment, the expression level of miR-198 is up-regulated in PC versus NP and in PC versus CP.

In a particular embodiment, the expression level of miR-614 is up-regulated in PC versus NP and in PC versus CP.

In a particular embodiment, the expression level of miR-122 is up-regulated in PC versus CP.

In one embodiment, miR-93* as a biomarker is claimed only in combination with another miR as a biomarker, such as miR-614.

In one embodiment, miR-122 as a biomarker is claimed only in combination with another miR as a biomarker, such as miR-614.

In one embodiment, miR-198 as a biomarker is claimed only in combination with another miR as a biomarker, such as miR-411.

In a further aspect, the present invention discloses miRNA biomarkers that are significantly differentially expressed between two conditions of the pancreas.

In one embodiment, said miRNA biomarkers may be used to distinguish between normal pancreas and pancreatic carcinoma, and comprises one or more miRNAs selected from the group of hsa-miR-198, hsa-miR-34c-5p, hsa-miR-21, hsa-miR-708, hsa-miR-614, hsa-miR-196b, hsa-miR-939, hsa-miR-148a, hsa-miR-801, hsa-miR-886-5p, hsa-miR-210, hsa-miR-190b, hsa-miR-142-3p, hsa-miR-130b*, hsa-miR-649, hsa-miR-30a*, hsa-miR-650, hsa-miR-492, hsa-miR-922, hsa-miR-31, hsa-miR-219-1-3p, hsa-miR-432*, hsa-miR-130b, hsa-miR-100*, hsa-miR-222*, hsa-miR-375, hsa-miR-135b*, hsa-miR-592, hsa-miR-494, hsa-miR-148a*, hsa-miR-635, hsa-miR-598, hsa-miR-622, hsa-miR-877, hsa-miR-875-5p, hsa-miR-451, hsa-miR-891a, hsa-miR-509-5p, hsa-miR-518d-3p, hsa-miR-648, hsa-miR-449b, hsa-miR-141*, hsa-miR-643, hsa-miR-575, hsa-miR-193b*, hsa-miR-217, hsa-miR-154*, hsa-miR-34b*, hsa-miR-7-2*, hsa-miR-147b, hsa-miR-584, hsa-miR-449a, hsa-miR-411*, hsa-miR-589*, hsa-miR-216b, hsa-miR-379*, hsa-miR-216a, hsa-miR-219-5p, hsa-miR-486-3p, hsa-miR-153, hsa-miR-143*, hsa-miR-542-5p, hsa-miR-644, hsa-miR-944, hsa-miR-129-5p, hsa-miR-19a*, hsa-miR-377*, hsa-miR-640, hsa-miR-383, hsa-miR-208, hsa-miR-566, hsa-miR-200c*, hsa-miR-147, hsa-miR-374a*, hsa-miR-92b*, hsa-miR-888, hsa-miR-205, hsa-miR-129-3p, hsa-miR-499-5p, hsa-miR-194*, hsa-miR-543 and hsa-miR-554.

In one embodiment, said miRNA biomarkers may be used to distinguish between chronic pancreatitis and pancreas cancer, and comprises one or more miRNAs selected from the group of hsa-miR-614, hsa-miR-492, hsa-miR-622, hsa-miR-135b*, hsa-miR-196b, hsa-miR-198, hsa-miR-516a-3p, hsa-miR-122, hsa-miR-509-5p, hsa-miR-147b, hsa-miR-148a, hsa-miR-648, hsa-miR-643, hsa-miR-125b-2*, hsa-miR-432*, hsa-miR-575, hsa-miR-520c-3p, hsa-miR-584, hsa-miR-377*, hsa-miR-148a*, hsa-miR-891a, hsa-miR-337-3p, hsa-miR-154*, hsa-miR-379*, hsa-miR-411*, hsa-miR-205, hsa-miR-208, hsa-miR-493*, hsa-miR-7-2*, hsa-miR-512-3p, hsa-miR-193b* and hsa-miR-374a.

In one embodiment, said miRNA biomarkers may be used to distinguish between ampullary adenocarcinoma and pancreas cancer, and comprises one or more miRNAs selected from the group of hsa-miR-194*, hsa-miR-187, hsa-miR-654-5p, hsa-miR-552 and hsa-miR-205.

In one embodiment, said miRNA biomarkers may be used to distinguish between normal pancreas and ampullary adenocarcinoma, and comprises one or more miRNAs selected from the group of hsa-miR-198, hsa-miR-10a, hsa-miR-650, hsa-miR-34c-5p, hsa-miR-30a*, hsa-miR-492, hsa-miR-148a, hsa-miR-30e*, hsa-miR-801, hsa-miR-614, hsa-miR-649, hsa-miR-143, hsa-miR-323-3p, hsa-miR-939, hsa-miR-130b*, hsa-miR-335, hsa-miR-30c, hsa-miR-31, hsa-miR-147b, hsa-miR-130b, hsa-miR-210, hsa-miR-922, hsa-miR-622, hsa-miR-548b-5p, hsa-miR-142-3p, hsa-miR-891a, hsa-miR-196b, hsa-miR-135b*, hsa-miR-133b, hsa-miR-590-5p, hsa-miR-494, hsa-miR-432*, hsa-miR-133a, hsa-miR-190b, hsa-miR-135b, hsa-miR-548d-5p, hsa-miR-598, hsa-miR-923, hsa-miR-143*, hsa-miR-604, hsa-miR-148a*, hsa-miR-411*, hsa-miR-7-2*, hsa-miR-551b*, hsa-miR-644, hsa-miR-379*, hsa-miR-639, hsa-miR-643, hsa-miR-487b, hsa-miR-575, hsa-miR-375, hsa-miR-635, hsa-miR-187, hsa-miR-875-5p, hsa-miR-154*, hsa-miR-888, hsa-miR-937, hsa-miR-203, hsa-miR-449b, hsa-miR-640, hsa-miR-147, hsa-miR-518d-3p, hsa-miR-648, hsa-miR-33a*, hsa-miR-656, hsa-miR-129-3p, hsa-miR-217, hsa-miR-153, hsa-miR-654-5p, hsa-miR-193b*, hsa-miR-451, hsa-miR-219-1-3p, hsa-miR-616*, hsa-miR-490-3p, hsa-miR-584, hsa-miR-889, hsa-miR-589*, hsa-miR-628-3p, hsa-miR-509-5p, hsa-miR-216a, hsa-miR-216b, hsa-miR-449a, hsa-miR-208, hsa-miR-129-5p, hsa-miR-377*, hsa-miR-486-3p, hsa-miR-455-3p, hsa-miR-184, hsa-miR-672, hsa-miR-19a*, hsa-miR-219-5p, hsa-miR-154, hsa-miR-518e, hsa-miR-374a*, hsa-miR-373, hsa-miR-582-3p, hsa-miR-124, hsa-let-7a*, hsa-miR-551b, hsa-miR-122, hsa-miR-543, hsa-miR-337-3p, hsa-miR-493*, hsa-miR-944, hsa-miR-552, hsa-miR-497*, hsa-miR-513-3p, hsa-miR-554 and hsa-miR-330-5p.

In one embodiment, said miRNA biomarkers may be used to distinguish between chronic pancreatitis and ampullary adenocarcinoma, and comprises one or more miRNAs selected from the group of hsa-miR-492, hsa-miR-622, hsa-miR-614, hsa-miR-147b, hsa-miR-135b*, hsa-miR-215, hsa-miR-194*, hsa-miR-135b, hsa-miR-203, hsa-miR-194, hsa-miR-192, hsa-miR-516a-3p, hsa-miR-133a, hsa-miR-196b, hsa-miR-891a, hsa-miR-133b, hsa-miR-649, hsa-miR-654-5p, hsa-miR-122, hsa-miR-411*, hsa-miR-125b-2*, hsa-miR-490-3p, hsa-miR-379*, hsa-miR-187, hsa-miR-450b-5p, hsa-miR-7-2*, hsa-miR-656, hsa-miR-337-3p, hsa-miR-575, hsa-miR-432*, hsa-miR-493*, hsa-miR-937, hsa-miR-888, hsa-miR-376b, hsa-miR-520c-3p, hsa-miR-497*, hsa-miR-518e, hsa-miR-129-3p, hsa-miR-512-3p, hsa-miR-648, hsa-miR-639, hsa-miR-377*, hsa-miR-154*, hsa-miR-208, hsa-miR-143*, hsa-miR-635, hsa-miR-644, hsa-miR-147, hsa-miR-509-5p, hsa-miR-518f, hsa-miR-922, hsa-miR-584, hsa-miR-148a*, hsa-miR-552, hsa-miR-154 and hsa-miR-543.

In one embodiment, said miRNA biomarkers may be used to distinguish between normal pancreas and chronic pancreatitis, and comprises one or more miRNAs selected from the group of hsa-miR-194*, hsa-miR-141*, hsa-miR-198, hsa-miR-130b*, hsa-miR-650, hsa-miR-219-1-3p and hsa-miR-766.

The miRNA biomarkers as disclosed herein may in one embodiment be used (or measured; correlated) alone.

The miRNA biomarkers as disclosed herein may in another embodiment be used in combination, comprising at least two miRNA biomarkers.

It follows, that the combination of miRNA biomarkers as disclosed herein may in one embodiment consist of 2 miRNAs, such as 3 miRNAs, for example 4 miRNAs, such as miRNAs, for example 6 miRNAs, such as 7 miRNAs, for example 8 miRNAs, such as 9 miRNAs, for example 10 miRNAs, such as 11 miRNAs, for example 12 miRNAs, such as 13 miRNAs, for example 14 miRNAs, such as 15 miRNAs, for example 16 miRNAs, such as 17 miRNAs, for example 18 miRNAs, such as 19 miRNAs, for example 20 miRNAs, as selected from the deregulated miRNA biomarkers disclosed herein.

The combination of miRNA biomarkers as disclosed may in another embodiment consist of less than 10 miRNAs, such as less than 9 miRNAs, for example less than 8 miRNAs, such as less than 7 miRNAs, for example less than 6 miRNAs, such as less than 5 miRNAs, for example less than 4 miRNAs, such as less than 3 miRNAs.

In a particular embodiment, the miRNA biomarker according to the present invention is not selected from the group consisting of miR-121, miR-93, miR-93*, miR-196b, miR-196a, miR-217, miR-21, miR-155, and miRNA selected from miR-205, miR-29c, miR-miR-216, miR-217, miR-375, miR-143, miR-145, miR-146a, miR-148a, miR-196b, miR-96, miR-31, miR-210, miR-148b, miR-196a, miR-141, miR-18a, miR-203, miR-150, miR-155, miR-130b, miR-221, miR-222, miR-223 and miR-224.

### Classifier

Classifiers are relationships between sets of input variables, usually known as features, and discrete output variables, known as classes. Classes are often centered on the key questions of who, what, where and when. A classifier can intuitively be thought of as offering an opinion about whether, for instance, an individual associated with a given feature set is a member of a given class.

In other words, a classifier is a predictive model that attempts to describe one column (the label) in terms of others (the attributes). A classifier is constructed from data where the label is known, and may be later applied to predict label values for new data where the label is unknown. Internally, a classifier is an algorithm or mathematical formula that predicts one discrete value for each input row. For example, a classifier built from a dataset of iris flowers could predict the type of a presented iris given the length and width of its petals and stamen. Classifiers may also produce probability estimates for each value of the label. For example, a classifier built from a dataset of cars could predict the probability that a specific car was built in the United States.

### Sensitivity and Specificity

Sensitivity and specificity are statistical measures of the performance of a binary classification test. The sensitivity (also called recall rate in some fields) measures the proportion of actual positives which are correctly identified as such (i.e. the percentage of sick people who are identified as having the condition); and the specificity measures the proportion of negatives which are correctly identified (i.e. the percentage of well people who are identified as not having the condition). They are closely related to the concepts of type I and type II errors.

For any test, there is usually a trade-off between each measure. For example in a manufacturing setting in which one is testing for faults, one may be willing to risk discarding functioning components (low specificity), in order to increase the chance of identifying nearly all faulty components (high sensitivity). This trade-off can be represented graphically using a ROC curve.

$$sensitivity = \frac{number of True Positives}{number of True Positives + number of False Negatives}$$

A sensitivity of 100% means that the test recognizes all sick people as such. Thus in a high sensitivity test, a negative result is used to rule out the disease.

Sensitivity alone does not tell us how well the test predicts other classes (that is, about the negative cases). In the binary classification, as illustrated above, this is the corresponding specificity test, or equivalently, the sensitivity for the other classes. Sensitivity is not the same as the positive predictive value (ratio of true positives to combined true and false positives), which is as much a statement about the proportion of actual positives in the population being tested as it is about the test.

The calculation of sensitivity does not take into account indeterminate test results. If a test cannot be repeated, the options are to exclude indeterminate samples from analyses (but the number of exclusions should be stated when quoting sensitivity), or, alternatively, indeterminate samples can be treated as false negatives (which gives the worst-case value for sensitivity and may therefore underestimate it).

$$specificity = \frac{number of True Negatives}{number of True Negatives + number of False Positives}$$

A specificity of 100% means that the test recognizes all healthy people as healthy. Thus a positive result in a high specificity test is used to confirm the disease. The maximum is trivially achieved by a test that claims everybody healthy regardless of the true condition. Therefore, the specificity alone does not tell us how well the test recognizes positive cases. We also need to know the sensitivity of the test to the class, or equivalently, the specificities to the other classes. A test with a high specificity has a low Type I error rate.

Specificity is sometimes confused with the precision or the positive predictive value, both of which refer to the fraction of returned positives that are true positives. The distinction is critical when the classes are different sizes. A test with very high specificity can have very low precision if there are far more true negatives than true positives, and vice versa.

The accuracy of a measurement system is the degree of closeness of measurements of a quantity to its actual (true) value. The precision of a measurement system, also called reproducibility or repeatability, is the degree to which repeated measurements under unchanged conditions show the same results.

Accuracy is also used as a statistical measure of how well a binary classification test correctly identifies or excludes a condition. That is, the accuracy is the proportion of true results (both true positives and true negatives) in the population. It is a parameter of the test:

$$accuracy = \frac{number of true positives + number of true negatives}{\begin{matrix}numbers of true positives + false positives + \\ false negatives + true negatives\end{matrix}}$$

An accuracy of 100% means that the measured values are exactly the same as the given values.

On the other hand, precision is defined as the proportion of the true positives against all the positive results (both true positives and false positives)

$$precision = \frac{number of true positives}{number of true positives + false positives}$$

miRNA Classifier of the Present Invention

The miRNA classifiers according to the present invention are the relationships between sets of input variables, i.e. the miRNA expression in a sample of an individual, and discrete output variables, i.e. distinction between e.g. a cancerous and non-cancerous condition of the pancreas. Thus, the classifier assigns a given sample to a given class with a given probability.

Distinction, differentiation or characterisation of a sample is used herein as being capable of predicting with a high sensitivity and specificity if a given sample of unknown diagnosis belongs to one of two classes (two-way classifier).

In one aspect, the miRNA classifier is a two-way classifier capable of predicting with an adequate sensitivity and specificity if a given sample of unknown diagnosis belongs to the combined class of pancreatic carcinoma and ampullary adenocarcinoma or the combined class of normal pancreas and chronic pancreatitis, wherein said miRNA classifier comprises or consists of one or more miRNAs selected from the group consisting of miR-198, miR-34c-5p, miR-614, miR-492, miR-10a, miR-622, miR-196b, miR-210, miR-939, miR-649, miR-801, miR-135b*, miR-148a, miR-194*, miR-21, miR-708, miR-222, miR-30a* and miR-323-3p.

In one embodiment, said two-way classifier according to the present invention does not comprise miR-801. In one embodiment, said two-way classifier according to the present invention does not comprise miR-21.

In another aspect, the miRNA classifier is a two-way classifier capable of predicting with an adequate sensitivity and specificity if a given sample of unknown diagnosis belongs to the combined class of pancreatic carcinoma and ampullary adenocarcinoma or the combined class of normal pancreas and chronic pancreatitis, wherein said miRNA classifier comprises or consists of one or more miRNAs selected from the group consisting of miR-122, miR-135b, miR-135b*, miR-136*, miR-186, miR-196b, miR-198, miR-203, miR-222, miR-23a, miR-34c-5p, miR-451, miR-490-3p, miR-492, miR-509-5p, miR-571, miR-614, miR-622 and miR-939.

This particular miRNA classifier separates samples containing PC or A-AC cells from non-neoplastic tissue samples (NP or CP) with a sensitivity of 0.985, a positive predictive value of 0.978 and an accuracy of 0.969 (See example).

In a particular embodiment, the miRNA classifier is a two-way classifier capable of predicting with an adequate sensitivity and specificity if a given sample of unknown diagnosis belongs to the combined class of either pancreatic carcinoma and ampullary adenocarcinoma or to the combined class of normal pancreas and chronic pancreatitis.

Platt's probabilistic outputs for Support Vector Machines (Platt, J. in Smola, A. J, et al. (eds.) Advances in large margin classifiers. Cambridge, 2000; incorporated herein by reference) is useful for applications that require posterior class probabilities. Also incorporated by reference herein is Platt J. Advances in Large Classifiers. Cambridge, Mass.: MIT Press, 1999.

The output of the two-way miRNA classifier is given as a probability of belonging to either class of between 0-1 (prediction probability). If the value for a sample is 0.5, no prediction is made. A number or value of between 0.51 to 1.0 for a given sample means that the sample is predicted to belong to the class in question, e.g. NP; and the corresponding value of 0.0 to 0.49 for the second class in question, e.g. PC, means that the sample is predicted not to belong to the class in question.

In one embodiment, the prediction probabilities for a sample to belong to a certain class is a number falling in the range of from 0 to 1, such as from 0.0 to 0.1, for example 0.1 to 0.2, such as 0.2 to 0.3, for example 0.3 to 0.4, such as 0.4 to 0.49, for example 0.5, such as 0.51 to 0.6, for example 0.6 to 0.7, such as 0.7 to 0.8, for example 0.8 to 0.9, such as 0.9 to 1.0.

In one embodiment, the prediction probability for a sample to belong to the NP class is a number falling in the range of from 0 to 0.49, 0.5 or from 0.51 to 1.0. In another embodiment, the prediction probability for a sample to belong to the PC class is a number between from 0 to 0.49, 0.5 or between from 0.51 to 1.0.

The classifier according to the present invention may in one embodiment consist of 2 miRNAs, such as 3 miRNAs, for example 4 miRNAs, such as 5 miRNAs, for example 6 miRNAs, such as 7 miRNAs, for example 8 miRNAs, such as 9 miRNAs, for example 10 miRNAs, such as 11 miRNAs, for example 12 miRNAs, such as 13 miRNAs, for example 14 miRNAs, such as 15 miRNAs, for example 16 miRNAs, such as 17 miRNAs, for example 18 miRNAs, such as 19 miRNAs selected from the group consisting of miR-198, miR-34c-5p, miR-614, miR-492, miR-10a, miR-622, miR-196b, miR-210, miR-939, miR-649, miR-801, miR-135b*, miR-148a, miR-194*, miR-21, miR-708, miR-222, miR-30a* and miR-323-3p.

The classifier according to the present invention may in another embodiment consist of 2 miRNAs, such as 3 miRNAs, for example 4 miRNAs, such as 5 miRNAs, for example 6 miRNAs, such as 7 miRNAs, for example 8 miRNAs, such as 9 miRNAs, for example 10 miRNAs, such as 11 miRNAs, for example 12 miRNAs, such as 13 miRNAs, for example 14 miRNAs, such as 15 miRNAs, for example 16 miRNAs, such as 17 miRNAs, for example 18 miRNAs, such as 19 miRNAs selected from the group consisting of hsa-miR-122, hsa-miR-135b, hsa-miR-135b*, hsa-miR-136*, hsa-miR-186, hsa-miR-196b, hsa-miR-198, hsa-miR-203, hsa-miR-222, hsa-miR-23a, hsa-miR-34c-5p, hsa-miR-451, hsa-miR-490-3p, hsa-miR-492, hsa-miR-509-5p, hsa-miR-571, hsa-miR-614, hsa-miR-622 and hsa-miR-939.

In one aspect, the present invention relates to a two-way miRNA classifier for characterising a sample obtained from an individual, wherein said miRNA classifier comprises or consists of one or more miRNAs selected from the group consisting of miR-198, miR-34c-5p, miR-614, miR-492, miR-10a, miR-622, miR-196b, miR-210, miR-939, miR-649, miR-801, miR-135b*, miR-148a, miR-194*, miR-21, miR-708, miR-222, miR-30a* and miR-323-3p, and distinguishes the combined class of pancreatic carcinoma and ampullary adenocarcinoma from the combined class of normal pancreas and chronic pancreatitis, wherein said distinction is given as a prediction probability for said sample of belonging to either class, said probability being a number falling in the range of from 0 to 1.

In one embodiment, the two-way miRNA classifier comprises miR-614.

In another aspect, the present invention relates to a two-way miRNA classifier for characterising a sample obtained from an individual, wherein said miRNA classifier comprises or consists of one or more miRNAs selected from the group consisting of miR-122, miR-135b, miR-135b*, miR-136*, miR-186, miR-196b, miR-198, miR-203, miR-222, miR-23a, miR-34c-5p, miR-451, miR-490-3p, miR-492, miR-509-5p, miR-571, miR-614, miR-622 and miR-939, and distinguishes the combined class of pancreatic carcinoma and ampullary adenocarcinoma from the combined class of normal pancreas and chronic pancreatitis, wherein said distinction is given as a prediction probability for said sample of belonging to either class, said probability being a number falling in the range of from 0 to 1.

The latter two-way miRNA classifier according to the present invention, when using all 19 miRNAs and with a model complexity of around 3.5, has a sensitivity of 0.985, a positive predictive value 0.978 and an accuracy of 0.969.

In one embodiment, the two-way miRNA classifier further comprises one or more additional miRNAs selected from the deregulated miRNA biomarkers as disclosed herein above.

In one embodiment, the two-way miRNA classifiers further comprises one or more additional miRNAs, such as 1 additional miRNA, for example 2 additional miRNAs, such as 3 additional miRNA, for example 4 additional miRNAs, such as 5 additional miRNA, for example 6 additional miRNAs, such as 7 additional miRNA, for example 8 additional miRNAs, such as 9 additional miRNA, for example 10 additional miRNAs, such as 11 additional miRNA, for example 12 additional miRNAs, such as 13 additional miRNA, for example 14 additional miRNAs, such as 15 additional miRNAs, for example 16 additional miRNAs, such as 17 additional miRNA, for example 18 additional miRNAs, such as 19 additional miRNAs, for example 20 additional miRNAs selected from the deregulated miRNA biomarkers as disclosed herein above.

In a particular embodiment, the two-way miRNA classifier does not comprise one or more of the miRNAs selected from the group consisting of mir-121, miR-93, miR-93*, miR-196b, miR-196a, miR-217, miR-21, miR-155, and miRNA selected from miR-205, miR-29c, miR-216, miR-217, miR-375, miR-143, miR-145, miR-146a, miR-148a, miR-miR-196b, miR-96, miR-31, miR-210, miR-148b, miR-196a, miR-141, miR-18a, miR-203, miR-150, miR-155, miR-130b, miR-221, miR-222, miR-223 and miR-224.

In an embodiment, an alteration of the expression profile or signature of one or more of the miRNAs of the two-way miRNA classifier according to the present invention is associated with the sample being classified as pancreatic cancer and/or AAC. In an embodiment, an alteration of the expression profile or signature of one or more of the miRNAs of the two-way miRNA classifier is associated with the sample being classified as normal pancreas and/or chronic pancreatitis.

In one embodiment, the present invention relates to a two-way miRNA classifier for characterising a sample obtained from an individual, wherein said miRNA classifier comprises or consists of one or more miRNAs selected from the group consisting of miR-198, miR-34c-5p, miR-614, miR-492, miR-10a, miR-622, miR-196b, miR-210, miR-939, miR-649, miR-801, miR-135b*, miR-148a, miR-194*, miR-21, miR-708, miR-222, miR-30a* and miR-323-3p, and distinguishes the combined class of pancreatic carcinoma and ampullary adenocarcinoma from the combined class of normal pancreas and chronic pancreatitis.

In another embodiment, the present invention relates to a two-way miRNA classifier for characterising a sample obtained from an individual, wherein said miRNA classifier comprises or consists of one or more miRNAs selected from the group consisting of miR-122, miR-135b, miR-135b*, miR-136*, miR-186, miR-196b, miR-198, miR-203, miR-222, miR-23a, miR-34c-5p, miR-451, miR-490-3p, miR-492, miR-509-5p, miR-571, miR-614, miR-622 and miR-939, and distinguishes the combined class of pancreatic carcinoma and ampullary adenocarcinoma from the combined class of normal pancreas and chronic pancreatitis.

The miRNA classifiers disclosed herein in a particular embodiment has a sensitivity of at least 80%, such as at least 81%, for example at least 82%, such as at least 83%, for example at least 84%, such as at least 85%, for example at least 86%, such as at least 87%, for example at least 88%, such as at least 89%, for example at least 90%, such as at least 91%, for example at least 92%, such as at least 93%, for example at least 94%, such as at least 95%.

The miRNA classifiers disclosed herein in a particular embodiment has an accuracy of at least 80%, such as at least 81%, for example at least 82%, such as at least 83%, for example at least 84%, such as at least 85%, for example at least 86%, such as at least 87%, for example at least 88%, such as at least 89%, for example at least 90%, such as at least 91%, for example at least 92%, such as at least 93%, for example at least 94%, such as at least 95%.

The miRNA classifiers disclosed herein in a particular embodiment has a specificity of at least 80%, such as at least 81%, for example at least 82%, such as at least 83%, for example at least 84%, such as at least 85%, for example at least 86%, such as at least 87%, for example at least 88%, such as at least 89%, for example at least 90%, such as at least 91%, for example at least 92%, such as at least 93%, for example at least 94%, such as at least 95%.

The miRNA classifiers disclosed herein in a particular embodiment has a negative predictive value for malignancies of at least 80%, such as at least 81%, for example at least 82%, such as at least 83%, for example at least 84%, such as at least 85%, for example at least 86%, such as at least 87%, for example at least 88%, such as at least 89%, for example at least 90%, such as at least 91%, for example at least 92%, such as at least 93%, for example at least 94%, such as at least 95%.

The miRNA classifiers disclosed herein in a particular embodiment has a positive predictive value for malignancies of at least 80%, such as at least 81%, for example at least 82%, such as at least 83%, for example at least 84%, such as at least 85%, for example at least 86%, such as at least 87%, for example at least 88%, such as at least 89%, for example at least 90%, such as at least 91%, for example at least 92%, such as at least 93%, for example at least 94%, such as at least 95%.

The miRNA classifiers disclosed herein in a particular embodiment has a positive predictive value or a negative predictive value for malignancies of between 80-85%, such as 85-90%, for example 90-95%, such as 95-96%, for example 96-97%, such as 97-98%, for example 98-99%, such as 99-100%.

Methods for Diagnosis Employing the miRNA Classifier and/or Biomarkers of the Present Invention

The invention in one aspect relates to a method for diagnosing if an individual has, or is at risk of developing, pancreatic carcinoma, comprising measuring the expression level of at least one miRNA in a sample obtained from said individual, wherein the at least one miRNA is selected from the group consisting of
i. miR-411 and miR-198; or
ii. miR-614 and miR-122; or
iii. miR-614 and miR-93*; or
iv. miR-198, miR-34c-5p, miR-614, miR-492, miR-10a, miR-622, miR-196b, miR-210, miR-939, miR-649, miR-801, miR-135b*, miR-148a, miR-194*, miR-21, miR-708, miR-222, miR-30a* and miR-323-3p; or
v. miR-122, miR-135b, miR-135b*, miR-136*, miR-186, miR-196b, miR-198, miR-203, miR-222, miR-23a, miR-34c-5p, miR-451, miR-490-3p, miR-492, miR-509-5p, miR-571, miR-614, miR-622 and miR-939,
   wherein the miRNA expression level, and/or the difference in the miRNA expression level, of at least one of said miRNAs is indicative of said individual having, or being at risk of developing, pancreatic carcinoma.

In is understood that said difference in miRNA expression level in a preferred embodiment is a relative difference between said miRNA's expression levels.

In one embodiment, said method further comprises the step of extracting RNA from a sample collected from an individual, by any means as disclosed herein elsewhere.

In one embodiment, said method further comprises the step of correlating the miRNA expression level of at least one of said miRNAs to a predetermined control level.

In one embodiment, said method further comprises the step of determining if said individual has, or is at risk of developing, pancreatic carcinoma.

In one embodiment, said method further comprises the step of obtaining a sample from an individual, by any means as disclosed herein elsewhere.

Said sample is in one particular embodiment a tissue sample from the pancreas of said individual. In another embodiment, said sample is a blood sample from said individual.

In one embodiment, said miRNA expression level is altered as compared to the expression level in a control sample. Said control sample may in one embodiment be normal pancreas and/or chronic pancreatitis.

In one embodiment, said pancreatic carcinoma is pancreatic adenocarcinoma. In another embodiment, said pancreatic carcinoma is ampullary adenocarcinoma. In a further embodiment, said pancreatic carcinoma comprises both pancreatic adenocarcinoma and ampullary adenocarcinoma.

In one embodiment, the at least one miRNA comprises or consists miR-411 and miR-198. In one embodiment, the at least one miRNA comprises or consists miR-614 and miR-122. In one embodiment, the at least one miRNA comprises or consists miR-614 and miR-93*.

The invention in one embodiment relates to a method for diagnosing if an individual has, or is at risk of developing, pancreatic carcinoma, comprising measuring the expression level of miR-411 and miR-198.

In one embodiment, the difference in the expression levels of miR-411 and miR-198 is calculated; and the difference in said expression levels of miR-411 and miR-198 is correlated to a condition of the pancreas. In one embodiment, this difference is altered in pancreatic cancer compared to normal pancreas and/or chronic pancreatitis.

In one embodiment the expression levels of miR-411 and of miR-198 are measured by QPCR and the difference in expression is calculated; wherein miR-198 is up-regulated in cancer (PC and A-AC) vs. control (NP and CP), and if the difference in the Ct level between miR-411 and miR-198 is between 0 to −5 the patient is diagnosed as having pancreatic cancer (PAC and/or AAC).

### Example

All individuals (having normal pancreas or pancreas cancer) has a very similar expression of miR-411; for example Ct=25. A PC patient has a high expression of miR-198 i.e. a low Ct-value of e.g. Ct=28. A healthy individual has a low expression of miR-198 i.e. a high Ct-value of e.g. Ct=34.

In one embodiment, the formula for diagnosing a PC patient is thus: 25 minus 28=−3, and the formula for a healthy individual is thus: 25−34=−9

The invention in one embodiment relates to a method for diagnosing if an individual has, or is at risk of developing, pancreatic carcinoma, comprising measuring the expression level of miR-614 and miR-122.

In one embodiment, the difference in the expression levels of miR-614 and miR-122 is calculated; and the difference in said expression levels of miR-614 and miR-122 is correlated to a condition of the pancreas. In one embodiment, this difference is altered in pancreatic cancer compared to normal pancreas and/or chronic pancreatitis.

In one embodiment the expression levels of miR-614 and of miR-122 are measured by QPCR and the difference in expression is calculated; wherein miR-614 is up-regulated in cancer (PC and A-AC) vs. control (NP and CP) and miR-122 is down-regulated in cancer (PC and A-AC) vs. control (NP and CP), and if the difference in the Ct level between miR-614 and miR-122 is between −2 to −12 the patient is diagnosed as having pancreatic cancer (PAC and/or AAC).

### Example

A PC patient (AAC and PAC) has a high expression of miR-614 i.e. a low Ct-value of e.g. Ct=28. A CP patient has a low expression of miR-614 i.e. a high Ct-value of e.g. Ct=32. A PC patient (AAC and PAC) has a low expression of miR-122 i.e. a low Ct-value of e.g. Ct=34. A CP patient has a high expression of miR-122 i.e. a low Ct-value of e.g. Ct=28.

In one embodiment, the formula for diagnosing a PC patient is thus: 25 minus 34=−6, and the formula for a CP patient is thus: 32−28=4.

The invention in one embodiment relates to a method for diagnosing if an individual has, or is at risk of developing, pancreatic carcinoma, comprising measuring the expression level of miR-614 and miR-93*

In one embodiment, the difference in the expression levels of miR-614 and miR-93* is calculated; and the difference in said expression levels of miR-614 and miR-93* is correlated to a condition of the pancreas. In one embodiment, this difference is altered in pancreatic cancer compared to normal pancreas and/or chronic pancreatitis.

In one embodiment the expression levels of miR-614 and of miR-93* are measured by QPCR and the difference in expression is calculated; wherein miR-614 is up-regulated in cancer (PC and A-AC) vs. control (NP and CP), and if the difference in the Ct level between miR-614 and miR-93* is between 0 to 6 the patient is diagnosed as having pancreatic cancer (PAC and/or AAC).

### Example

A PC patient (AAC and PAC) has a high expression of miR-614 i.e. a low Ct-value of e.g. Ct=28. A CP patient has a low expression of miR-614 i.e. a high Ct-value of e.g. Ct=34. All individuals (having normal pancreas or pancreas cancer) has a very similar expression of miR-93*; for example Ct=25.

In one embodiment, the formula for diagnosing a PC patient is thus: 28 minus 25=3, and the formula for a CP patient is thus: 34−25=9.

The invention in one embodiment relates to a method for diagnosing if an individual has, or is at risk of developing, pancreatic carcinoma, comprising measuring the expression level of at least one miRNA in a sample obtained from an individual, wherein said at least one miRNA is selected from the group consisting of miR-198, miR-34c-5p, miR-614, miR-492, miR-10a, miR-622, miR-196b, miR-210, miR-939, miR-649, miR-801, miR-135b*, miR-148a, miR-194*, miR-21, miR-708, miR-222, miR-30a* and miR-323-3p. In one embodiment, all of said miRNAs are measured.

In one embodiment, the expression level of one or more of miR-198, miR-34c-5p, miR-614, miR-492, miR-10a, miR-622, miR-196b, miR-210, miR-939, miR-649, miR-801, miR-135b*, miR-148a, miR-194*, miR-21, miR-708, miR-222, miR-30a* and miR-323-3p is altered in pancreatic cancer (PAC and/or AAC) compared to normal pancreas and/or chronic pancreatitis.

In another embodiment, the difference in expression level of one or more of miR-198, miR-34c-5p, miR-614, miR-492, miR-10a, miR-622, miR-196b, miR-210, miR-939, miR-649, miR-801, miR-135b*, miR-148a, miR-194*, miR-21, miR-708, miR-222, miR-30a* and miR-323-3p is altered in pancreatic cancer (PAC and/or AAC) compared to normal pancreas and/or chronic pancreatitis.

The invention in another embodiment relates to a method for diagnosing if an individual has, or is at risk of developing, pancreatic carcinoma, comprising measuring the expression level of at least one miRNA in a sample obtained from an individual, wherein said at least one miRNA is selected from the group consisting of miR-122, miR-135b, miR-135b*, miR-136*, miR-186, miR-196b, miR-198, miR-203, miR-222, miR-23a, miR-34c-5p, miR-451, miR-490-3p, miR-492, miR-509-5p, miR-571, miR-614, miR-622 and miR-939. In one embodiment, all of said miRNAs are measured.

In one embodiment, the expression level of one or more of miR-122, miR-135b, miR-135b*, miR-136*, miR-186, miR-196b, miR-198, miR-203, miR-222, miR-23a, miR-34c-5p, miR-451, miR-490-3p, miR-492, miR-509-5p, miR-571, miR-614, miR-622 and miR-939 is altered in pancreatic cancer (PAC and/or AAC) compared to normal pancreas and/or chronic pancreatitis.

In another embodiment, the difference in expression level of one or more of miR-122, miR-135b, miR-135b*, miR-136*, miR-186, miR-196b, miR-198, miR-203, miR-222, miR-23a, miR-34c-5p, miR-451, miR-490-3p, miR-492, miR-509-5p, miR-571, miR-614, miR-622 and miR-939 is altered in pancreatic cancer (PAC and/or AAC) compared to normal pancreas and/or chronic pancreatitis.

It follows, that any of the above-mentioned methods may further comprise the step of obtaining prediction probabilities of between 0-1.

In one embodiment, said method of diagnosing an individual comprises measuring the expression level of at least 2 miRNAs; for example 2 miRNAs, such as 3 miRNAs, for example 4 miRNAs, such as 5 miRNAs, for example 6 miRNAs, such as 7 miRNAs, for example 8 miRNAs, such as 9 miRNAs, for example 10 miRNAs, such as 11 miRNAs, for example 12 miRNAs, such as 13 miRNAs, for example 14 miRNAs, such as 15 miRNAs, for example 16 miRNAs, such as 17 miRNAs, for example 18 miRNAs, such as 19 miRNAs, as selected from the deregulated miRNAs disclosed herein.

In one embodiment, said method of diagnosing an individual further comprises measuring the expression level of one or more additional miRNAs, said miRNA being selected from the group consisting of hsa-miR-93, hsa-miR-93*, hsa-miR-411, hsa-miR-198, hsa-miR-34c-5p, hsa-miR-21, hsa-miR-708, hsa-miR-614, hsa-miR-196b, hsa-miR-939, hsa-miR-148a, hsa-miR-801, hsa-miR-886-5p, hsa-miR-210, hsa-miR-190b, hsa-miR-142-3p, hsa-miR-130b*, hsa-miR-649, hsa-miR-30a*, hsa-miR-650, hsa-miR-492, hsa-miR-922, hsa-miR-31, hsa-miR-219-1-3p, hsa-miR-432*, hsa-miR-130b, hsa-miR-100*, hsa-miR-222*, hsa-miR-222, hsa-miR-375, hsa-miR-135b*, hsa-miR-592, hsa-miR-494, hsa-miR-148a*, hsa-miR-635, hsa-miR-598, hsa-miR-622, hsa-miR-877, hsa-miR-875-5p, hsa-miR-451, hsa-miR-891a, hsa-miR-509-5p, hsa-miR-518d-3p, hsa-miR-648, hsa-miR-449b, hsa-miR-141*, hsa-miR-643, hsa-miR-575, hsa-miR-193b*, hsa-miR-217, hsa-miR-154*, hsa-miR-34b*, hsa-miR-7-2*, hsa-miR-147b, hsa-miR-584, hsa-miR-449a, hsa-miR-411*, has-miR-411, hsa-miR-589*, hsa-miR-216b, hsa-miR-379*, hsa-miR-216a, hsa-miR-219-5p, hsa-miR-486-3p, hsa-miR-153, hsa-miR-143*, hsa-miR-542-5p, hsa-miR-644, hsa-miR-944, hsa-miR-129-5p, hsa-miR-19a*, hsa-miR-377*, hsa-miR-640, hsa-miR-383, hsa-miR-208, hsa-miR-566, hsa-miR-200c*, hsa-miR-147, hsa-miR-374a*, hsa-miR-92b*, hsa-miR-888, hsa-miR-205, hsa-miR-129-3p, hsa-miR-499-5p, hsa-miR-194*, hsa-miR-543, hsa-miR-554, hsa-miR-141*, hsa-miR-766, hsa-miR-516a-3p, hsa-miR-215, hsa-miR-135b, hsa-miR-203, hsa-miR-194, hsa-miR-192, hsa-miR-133a, hsa-miR-133b, hsa-miR-654-5p, hsa-miR-154, hsa-miR-122, hsa-miR-125b-2*, hsa-miR-490-3p, hsa-miR-552, hsa-miR-187, hsa-miR-518f, hsa-miR-450b-5p, hsa-miR-656, hsa-miR-10a, hsa-miR-337-3p, hsa-miR-520c-3p, hsa-miR-493*, hsa-miR-512-3p, hsa-miR-374a, hsa-miR-30e*, hsa-miR-937, hsa-miR-376b, hsa-miR-639, hsa-miR-497*, hsa-miR-518e, hsa-miR-143, hsa-miR-323-3p, hsa-miR-335, hsa-miR-30c, hsa-miR-548b-5p, hsa-miR-590-5p, hsa-miR-548d-5p, hsa-miR-551b*, hsa-miR-487b, hsa-miR-33a*, hsa-miR-616*, hsa-miR-889, hsa-miR-628-3p, hsa-miR-455-3p, hsa-miR-184, hsa-miR-672, hsa-miR-373, hsa-miR-582-3p, hsa-miR-124, hsa-let-7a*, hsa-miR-551b, hsa-miR-513-3p and hsa-miR-330-5p.

In a further embodiment, any of the above-mentioned methods may be is used in combination with at least one additional diagnostic method.

Said at least one additional diagnostic method may in one embodiment be selected from the group consisting of CT (X-ray computed tomography), MRI (magnetic resonance imaging), Scintillation counting, Blood sample analysis, Ultrasound imaging, Cytology, Histology and Assessment of risk factors. These are described herein above.

In one embodiment, said at least one additional diagnostic method improves the sensitivity and/or specificity of the combined diagnostic outcome.

The invention in a further aspect relates to a method for expression profiling of a sample, comprising measuring at least one miRNA selected from the group of miR-411 and miR-198; miR-614 and miR-122; miR-614 and miR-93*; or miR-34c-5p, miR-492, miR-10a, miR-622, miR-196b, miR-210, miR-939, miR-649, miR-801, miR-135b*, miR-148a, miR-194*, miR-21, miR-708, miR-222, miR-30a* and miR-323-3p; and correlating said expression profile to a clinical condition selected from pancreatic carcinoma, pancreatic adenocarcinoma, ampullary adenocarcinoma and chronic pancreatitis.

The invention in a further aspect relates to a method for expression profiling of a sample, comprising measuring at least one miRNA selected from the group of miR-122, miR-135b, miR-135b*, miR-136*, miR-186, miR-196b, miR-198, miR-203, miR-222, miR-23a, miR-34c-5p, miR-451, miR-490-3p, miR-492, miR-509-5p, miR-571, miR-614, miR-622 and miR-939; and correlating said expression profile to a clinical condition selected from pancreatic carcinoma, pancreatic adenocarcinoma, ampullary adenocarcinoma and chronic pancreatitis.

A Model for Predicting a Diagnosis by Employing the miRNA Classifier of the Present Invention

In one aspect, the present invention relates to a model for predicting the diagnosis of an individual, comprising
i) providing a set of input data to the miRNA classifier according to the present invention, and
ii) determining if said individual has a condition selected from the combined class of pancreatic carcinoma and ampullary adenocarcinoma and the combined class of normal pancreas and chronic pancreatitis.

In one embodiment, said input data comprises or consists of the miRNA expression profile of one or more of miR-198, miR-34c-5p, miR-614, miR-492, miR-10a, miR-622, miR-196b, miR-210, miR-939, miR-649, miR-801, miR-135b*, miR-148a, miR-194*, miR-21, miR-708, miR-222, miR-30a* and miR-323-3p.

In another embodiment, said input data comprises or consists of the miRNA expression profile of one or more of miR-122, miR-135b, miR-135b*, miR-136*, miR-186, miR-196b, miR-198, miR-203, miR-222, miR-23a, miR-34c-5p, miR-451, miR-490-3p, miR-492, miR-509-5p, miR-571, miR-614, miR-622 and miR-939.

In a further embodiment, the model according to the present invention further comprises one or more additional miRNAs selected from the deregulated miRNA biomarkers disclosed herein.

In one embodiment, said additional miRNAs comprise 1 additional miRNA, for example 2 additional miRNAs, such as 3 additional miRNA, for example 4 additional miRNAs, such as 5 additional miRNA, for example 6 additional miRNAs, such as 7 additional miRNA, for example 8 additional miRNAs, such as 9 additional miRNA, for example 10 additional miRNAs, such as 11 additional miRNA, for example 12 additional miRNAs, such as 13 additional miRNA, for example 14 additional miRNAs, such as 15 additional miRNA, for example 16 additional miRNAs, such as 17 additional miRNA, for example 18 additional miRNAs, such as 19 additional miRNA, for example 20 additional miRNAs selected from the deregulated miRNA according to the present invention.

### Sample Type

The sample according to the present invention is extracted from an individual and used for miRNA profiling for the subsequent diagnosis of a condition of the pancreas.

The sample may be collected from an individual or a cell culture, preferably an individual. The individual may be any animal, such as a mammal, including human beings. In a preferred embodiment, the individual is a human being.

In a particular embodiment, the sample is taken from the pancreas of a human being. In such an instance, the sample may be denoted a tissue sample. Said pancreas sample preferably comprises pancreatic cells. If a cancer of sorts is present in the pancreas, the sample preferably comprises pancreatic cancer cells.

The tissue sample further comprises cells of the desmoplastic stroma surrounding the tumour, e.g. fibroblasts, pancreatic stellate cells, inflammatory cells (e.g. macrophages and neutrofils) and endothelial cells.

In another particular embodiment, the sample is a blood sample drawn from a human being.

### Sample Collection

In one embodiment, the sample is collected from the pancreas of an individual by any available means, such as by fine-needle aspiration (FNA) using a needle with a maximum diameter of 1 mm; by core needle aspiration using a needle with a maximum diameter of above 1 mm (also called coarse needle aspiration or biopsy, large needle aspiration or large core aspiration); by biopsy; by cutting biopsy; by open biopsy; a surgical sample; or by any other means known to the person skilled in the art. In another embodiment, the sample is collected from an in vitro cell culture.

In a particular embodiment, the sample is a fine-needle aspirate from an individual. The fine-needle aspiration may be performed using a needle with a diameter of between 0.2 to 1.0 mm, such as 0.2 to 0.3 mm, for example 0.3 to 0.4 mm, such as 0.4 to 0.5 mm, for example 0.5 to 0.6 mm, such as 0.6 to 0.7 mm, for example 0.7 to 0.8 mm, such as 0.8 to 0.9 mm, for example 0.9 to 1.0 mm in diameter.

Said fine-needle aspiration may in one embodiment be a single fine-needle aspiration, or may in another embodiment comprise multiple fine-needle aspirations.

The diameter of the needle is indicated by the needle gauge. Various needle lengths are available for any given gauge. Needles in common medical use range from 7 gauge (the largest) to 33 (the smallest) on the Stubs scale. Although reusable needles remain useful for some scientific applications, disposable needles are far more common in medicine. Disposable needles are embedded in a plastic or aluminium hub that attaches to the syringe barrel by means of a press-fit (Luer) or twist-on (Luer-lock) fitting.

The fine-needle aspiration is in one embodiment performed using a needle gauge of between 20 to 33, such as needle gauge 20, for example needle gauge 21, such as needle gauge 22, for example needle gauge 23, such as needle gauge 24, for example needle gauge 25, such as needle gauge 26, for example needle gauge 27, such as needle gauge 28, for example needle gauge 29, such as needle gauge 30, for example needle gauge 31, such as needle gauge 32, for example needle gauge 33.

The fine-needle aspiration may in one embodiment be assisted, such as ultra-sound (US) guided fine-needle aspiration, x-ray guided fine-needle aspiration, endoscopic ultra-sound (EUS) guided fine-needle aspiration, Endobronchial ultrasound-guided fine-needle aspiration (EBUS), ultrasonographically guided fine-needle aspiration, stereotactically guided fine-needle aspiration, computed tomography (CT)-guided percutaneous fine-needle aspiration and palpation guided fine-needle aspiration.

The skin above the area to be biopsied may in one embodiment be swiped with an antiseptic solution and/or may be draped with sterile surgical towels. The skin, underlying fat, and muscle may in one embodiment be numbed with a local anesthetic. After the needle is placed into the mass, cells may be withdrawn by aspiration with a syringe.

In another embodiment, the sample is a blood sample extracted or drawn from an individual by any conventional method known to the skilled person. The blood may be drawn from a vein or an artery of an individual.

The sample extracted from an individual by any means as disclosed above may be transferred to a tube or container prior to analysis. The container may be empty, or may comprise a collection media of sorts.

The sample extracted from an individual by any means as disclosed above may be analysed essentially immediately, or it may be stored prior to analysis for a variable period of time and at various temperature ranges.

In one embodiment, the sample is stored at a temperature of between −200° C. to 37° C., such as between −200 to −100° C., for example −100 to −50° C., such as −50 to −25° C., for example −25 to −10° C., such as −10 to 0° C., for example 0 to 10° C., such as 10 to 20° C., for example 20 to 30° C., such as 30 to 37° C. prior to analysis.

In one embodiment, the sample is stored at −20° C. and/or −80° C.

In another embodiment, the sample is stored for between 15 minutes and 100 years prior to analysis, such as between 15 minutes and 1 hour, for example 1 to 2 hours, such as 2 to 5 hours, for example 5 to 10 hours, such as 10 to 24 hours, for example 24 hours to 48 hours, such as 48 to 72 hours, for example 72 to 96 hours, such as 4 to 7 days, such as 1 week to 2 weeks, such as 2 to 4 weeks, such as 4 weeks to 1 month, such as 1 month to 2 months, for example 2 to 3 months, such as 3 to 4 months, for example 4 to 5 months, such as 5 to 6 months, for example 6 to 7 months, such as 7 to 8 months, for example 8 to 9 months, such as 9 to 10 months, for example 10 to 11 months, such as 11 to 12 months, for example 1 year to 2 years, such as 2 to 3 years, for example 3 to 4 years, such as 4 to 5 years, for example 5 to 6 years, such as 6 to 7 years, for example 7 to 8 years, such as 8 to 9 years, for example 9 to 10 years, such as 10 to 20 years, for example 20 to 30 years, such as 30 to 40 years, for example 40 to 50 years, such as 50 to 75 years, for example 75 to 100 years prior to analysis.

In one embodiment, the sample is stored for a few days.

### Collection Media for Sample

A collection media according to the present invention is any media suitable for preserving and/or collecting a sample for immediate or later analysis.

In one embodiment, said collection media is a solution suitable for sample preservation and/or later retrieval of RNA (such as miRNA) from said sample.

In one embodiment, the collection media is an RNA preservation solution or reagent suitable for containing samples without the immediate need for cooling or freezing the sample, while maintaining RNA integrity prior to extraction of RNA (such as miRNA) from the sample. An RNA preservation solution or reagent may also be known as RNA stabilization solution or reagent or RNA recovery media, and may be used interchangeably herein. The RNA preservation solution may penetrate the harvested cells of the collected sample to retard RNA degradation to a rate dependent on the storage temperature.

The RNA preservation solution may be any commercially available solutions or it may be a solution prepared according to available protocols.

The commercially available RNA preservation solutions may for example be selected from RNAlater® (Ambion and Qiagen), PreservCyt medium (Cytyc Corp), PrepProtect™ Stabilisation Buffer (Miltenyi Biotec), Allprotect Tissue Reagent (Qiagen) and RNAprotect Cell Reagent (Qiagen). Protocols for preparing a RNA stabilizing solution may be retrieved from the internet (e.g. L. A. Clarke and M. D. Amaral: ‘Protocol for RNase-retarding solution for cell samples’, provided through The European Workin Group on CFTR Expression), or may be produced and/or optimized according to techniques known to the skilled person.

In another embodiment, the collection media will penetrate and lyse the cells of the sample immediately, including reagents and methods for isolating RNA (such as miRNA) from a sample that may or may not include the use of a spin column.

Said reagents and methods for isolating RNA (such as miRNA) is described herein below in the section ‘analysis of sample’.

Other collection media according to the present invention comprises any media such as water, sterile water, denatured water, saline solutions, buffers, PBS, TBS, Allprotect Tissue Reagent (Qiagen), cell culture media such as RPMI-1640, DMEM (Dulbecco's Modified Eagle Medium), MEM (Minimal Essential Medium), IMDM (Iscove's Modified Dulbecco's Medium), BGjB (Fitton-Jackson modification), BME (Basal Medium Eagle), Brinster's BMOC-3 Medium, CMRL Medium, CO₂-Independent Medium, F-10 and F-12 Nutrient Mixture, GMEM (Glasgow Minimum Essential Medium), IMEM (Improved Minimum Essential Medium), Leibovitz's L-15 Medium, McCoy's 5A Medium, MCDB 131 Medium, Medium 199, Opti-MEM, Waymouth's MB 752/1, Williams' Media E, Tyrode's solution, Belyakov's solution, Hanks' solution and other cell culture media known to the skilled person, tissue preservation media such as HypoThermosol®, CryoStor™ and Steinhardt's medium and other tissue preservation media known to the skilled person.

In another embodiment, said collection media is means for fixation (preservation) of said tissue sample; a tissue fixative, such as formalin (formaldehyde) or the like.

Types of tissue fixation includes heat fixation, chemical fixation (Crosslinking fixatives—Aldehydes; Precipitating fixatives—Alcohols; Oxidising agents; Mercurials; Picrates; HOPE (Hepes-glutamic acid buffer-mediated organic solvent protection effect) Fixative), and Frozen Sections.

In one embodiment, the fixation time may be between 1 to 7 calendar days; such as 1 day, 2 days, 3 days, 4 days, 5 days, 6 days or 7 days.

It follows that the invention may be carried out on formalin fixed paraffin embedded tissue blocks (FFPE).

### Sample Analysis

After the sample is collected, it is subjected to analysis. In one embodiment, the sample is initially used for isolating or extracting RNA according to any conventional methods known in the art; followed by an analysis of the miRNA expression in said sample.

### Extraction of RNA

The RNA isolated from the sample may be total RNA, mRNA, microRNA, tRNA, rRNA or any type of RNA.

Conventional methods and reagents for isolating RNA from a sample comprise High Pure miRNA Isolation Kit (Roche), Trizol (Invitrogen), Guanidinium thiocyanate-phenol-chloroform extraction, PureLink™ miRNA isolation kit (Invitrogen), PureLink Micro-to-Midi Total RNA Purification System (invitrogen), RNeasy kit (Qiagen), miRNeasy kit (Qiagen), Oligotex kit (Qiagen), phenol extraction, phenol-chloroform extraction, TCA/acetone precipitation, ethanol precipitation, Column purification, Silica gel membrane purification, PureYield™ RNA Midiprep (Promega), PolyATtract System 1000 (Promega), Maxwell® 16 System (Promega), SV Total RNA Isolation (Promega), geneMAG-RNA/DNA kit (Chemicell), TRI Reagent® (Ambion), RNAqueous Kit (Ambion), ToTALLY RNA™ Kit (Ambion), Poly(A)Purist™ Kit (Ambion) and any other methods, commercially available or not, known to the skilled person.

The RNA may be further amplified, cleaned-up, concentrated, DNase treated, quantified or otherwise analysed or examined such as by agarose gel electrophoresis, absorbance spectrometry or Bioanalyser analysis (Agilent) or subjected to any other post-extraction method known to the skilled person.

Methods for extracting and analysing an RNA sample are disclosed in Molecular Cloning, A Laboratory Manual (Sambrook and Russell (ed.), 3^{rd} edition (2001), Cold Spring Harbor Laboratory Press, Cold Spring Harbor, N.Y., USA.

### Microarray Analysis

The isolated RNA may be analysed by microarray analysis. In one embodiment, the expression level of one or more miRNAs is determined by the microarray technique.

A microarray is a multiplex technology that consists of an arrayed series of thousands of microscopic spots of DNA oligonucleotides or antisense miRNA probes, called features, each containing picomoles of a specific oligonucleotide sequence. This can be a short section of a gene or other DNA or RNA element that are used as probes to hybridize a DNA or RNA sample (called target) under high-stringency conditions. Probe-target hybridization is usually detected and quantified by fluorescence-based detection of fluorophore-labeled targets to determine relative abundance of nucleic acid sequences in the target. In standard microarrays, the probes are attached to a solid surface by a covalent bond to a chemical matrix (via epoxy-silane, amino-silane, lysine, polyacrylamide or others). The solid surface can be glass or a silicon chip, in which case they are commonly known as gene chip. DNA arrays are so named because they either measure DNA or use DNA as part of its detection system. The DNA probe may however be a modified DNA structure such as LNA (locked nucleic acid).

In one embodiment, the microarray analysis is used to detect microRNA, known as microRNA or miRNA expression profiling.

The microarray for detection of microRNA may be a microarray platform, wherein the probes of the microarray may be comprised of antisense miRNAs or DNA oligonucleotides. In the first case, the target is a labelled sense miRNA sequence, and in the latter case the miRNA has been reverse transcribed into cDNA and labelled.

The microarray for detection of microRNA may be a commercially available array platform, such as NCode™ miRNA Microarray Expression Profiling (Invitrogen), miRCURY LNA™ microRNA Arrays (Exiqon), microRNA Array (Agilent), μParaflo® Microfluidic Biochip Technology (LC Sciences), MicroRNA Profiling Panels (Illumina), Geniom® Biochips (Febit Inc.), microRNA Array (Oxford Gene Technology), Custom AdmiRNA™ profiling service (Applied Biological Materials Inc.), microRNA Array (Dharmacon—Thermo Scientific), LDA TaqMan analyses (Applied Biosystems), Taqman microRNA Array (Applied Biosystems) or any other commercially available array.

Microarray analysis may comprise all or a subset of the steps of RNA isolation, RNA amplification, reverse transcription, target labelling, hybridisation onto a microarray chip, image analysis and normalisation, and subsequent data analysis; each of these steps may be performed according to a manufacturers protocol.

It follows, that any of the methods as disclosed herein above e.g. for diagnosing of an individual may further comprise one or more of the steps of:
i) isolating miRNA from a sample,
ii) labelling of said miRNA,
iii) hybridising said labelled miRNA to a microarray comprising miRNA-specific probes to provide a hybridisation profile for the sample,
iv) performing data analysis to obtain a measure of the miRNA expression profile of said sample.

In another embodiment, the microarray for detection of microRNA is custom made.

A probe or hybridization probe is a fragment of DNA or RNA of variable length, which is used to detect in DNA or RNA samples the presence of nucleotide sequences (the target) that are complementary to the sequence in the probe. One example is a sense miRNA sequence in a sample (target) and an antisense miRNA probe. The probe thereby hybridizes to single-stranded nucleic acid (DNA or RNA) whose base sequence allows probe-target base pairing due to complementarity between the probe and target.

To detect hybridization of the probe to its target sequence, the probe or the sample is tagged (or labeled) with a molecular marker. Detection of sequences with moderate or high similarity depends on how stringent the hybridization conditions were applied—high stringency, such as high hybridization temperature and low salt in hybridization buffers, permits only hybridization between nucleic acid sequences that are highly similar, whereas low stringency, such as lower temperature and high salt, allows hybridization when the sequences are less similar. Hybridization probes used in microarrays refer to nucleotide sequences covalently attached to an inert surface, such as coated glass slides, and to which a mobile target is hybridized. Depending on the method the probe may be synthesised via phosphoramidite technology or generated by PCR amplification or cloning (older methods). To design probe sequences, a probe design algorithm may be used to ensure maximum specificity (discerning closely related targets), sensitivity (maximum hybridisation intensities) and normalised melting temperatures for uniform hybridisation.

### RT-QPCR

The isolated RNA may be analysed by quantitative (‘real-time’) PCR (QPCR). In one embodiment, the expression level of one or more miRNAs is determined by the quantitative polymerase chain reaction (QPCR) technique.

Real-time polymerase chain reaction, also called quantitative polymerase chain reaction (Q-PCR/qPCR/RT-QPCR) or kinetic polymerase chain reaction, is a technique based on the polymerase chain reaction, which is used to amplify and simultaneously quantify a targeted DNA molecule. It enables both detection and quantification (as absolute number of copies or relative amount when normalized to DNA input or additional normalizing genes) of a specific sequence in a DNA sample.

The procedure follows the general principle of polymerase chain reaction; its key feature is that the amplified DNA is quantified as it accumulates in the reaction in real time after each amplification cycle. Two common methods of quantification are the use of fluorescent dyes that intercalate with double-stranded DNA, and modified DNA oligonucleotide probes that fluoresce when hybridized with a complementary DNA. Frequently, real-time polymerase chain reaction is combined with reverse transcription polymerase chain reaction to quantify low abundance messenger RNA (mRNA), or miRNA, enabling a researcher to quantify relative gene expression at a particular time, or in a particular cell or tissue type.

In a real time PCR assay a positive reaction is detected by accumulation of a fluorescent signal. The Ct (cycle threshold) is defined as the number of cycles required for the fluorescent signal to cross the threshold (i.e. exceeds background level). Ct levels are inversely proportional to the amount of target nucleic acid in the sample (i.e. the lower the Ct level the greater the amount of target nucleic acid in the sample). Most real time assays undergo 40 cycles of amplification.

Cts<29 are strong positive reactions indicative of abundant target nucleic acid in the sample. Cts of 30-37 are positive reactions indicative of moderate amounts of target nucleic acid. Cts of 38-40 are weak reactions indicative of minimal amounts of target nucleic acid which could represent an infection state or environmental contamination. The QPCR may be performed using chemicals and/or machines from a commercially available platform.

The QPCR may be performed using QPCR machines from any commercially available platform; such as Prism, geneAmp or StepOne Real Time PCR systems (Applied Biosystems), LightCycler (Roche), RapidCycler (Idaho Technology), MasterCycler (Eppendorf), iCycler iQ system, Chromo 4 system, CFX, MiniOpticon and Opticon systems (Bio-Rad), SmartCycler system (Cepheid), RotorGene system (Corbett Lifescience), MX3000 and MX3005 systems (Stratagene), DNA Engine Opticon system (Qiagen), Quantica qPCR systems (Techne), InSyte and Syncrom cycler system (BioGene), DT-322 (DNA Technology), Exicycler Notebook Thermal cycler, TL998 System (lanlong), Line-Gene-K systems (Bioer Technology), or any other commercially available platform.

The QPCR may be performed using chemicals from any commercially available platform, such as NCode EXPRESS qPCR or EXPRESS qPCR (Invitrogen), Taqman or SYBR green qPCR systems (Applied Biosystems), Real-Time PCR reagents (Eurogentec), iTaq mix (Bio-Rad), qPCR mixes and kits (Biosense), and any other chemicals, commercially available or not, known to the skilled person.

The QPCR reagents and detection system may be probe-based, or may be based on chelating a fluorescent chemical into double-stranded oligonucleotides.

The QPCR reaction may be performed in a tube; such as a single tube, a tube strip or a plate, or it may be performed in a microfluidic card in which the relevant probes and/or primers are already integrated.

A Microfluidic card allows high throughput, parallel analysis of mRNA or miRNA expression patterns, and allows for a quick and cost-effective investigation of biological pathways. The microfluidic card may be a piece of plastic that is riddled with micro channels and chambers filled with the probes needed to translate a sample into a diagnosis. A sample in fluid form is injected into one end of the card, and capillary action causes the fluid sample to be distributed into the microchannels. The microfluidic card is then placed in an appropriate device for processing the card and reading the signal.

### Other Analysis Methods

The isolated RNA may be analysed by northern blotting. In one embodiment, the expression level of one or more miRNAs is determined by the northern blot technique.

A northern blot is a method used to check for the presence of a RNA sequence in a sample. Northern blotting combines denaturing agarose gel or polyacrylamide gel electrophoresis for size separation of RNA with methods to transfer the size-separated RNA to a filter membrane for probe hybridization. The hybridization probe may be made from DNA or RNA.

In yet another embodiment, the isolated RNA is analysed by nuclease protection assay.

The isolated RNA may be analysed by Nuclease protection assay.

Nuclease protection assay is a technique used to identify individual RNA molecules in a heterogeneous RNA sample extracted from cells. The technique can identify one or more RNA molecules of known sequence even at low total concentration. The extracted RNA is first mixed with antisense RNA or DNA probes that are complementary to the sequence or sequences of interest and the complementary strands are hybridized to form double-stranded RNA (or a DNA-RNA hybrid). The mixture is then exposed to ribonucleases that specifically cleave only single-stranded RNA but have no activity against double-stranded RNA. When the reaction runs to completion, susceptible RNA regions are degraded to very short oligomers or to individual nucleotides; the surviving RNA fragments are those that were complementary to the added antisense strand and thus contained the sequence of interest.

### Device

It is also an aspect of the present invention to provide a device for measuring the expression level of at least one miRNA in a sample, wherein said device comprises or consists of at least one probe or probe set for at least one miRNA selected from the group consisting of
i) miR-411 and miR-198,
ii) miR-614 and miR-122,
iii) miR-614 and miR-93*, or
iv) miR-198, miR-34c-5p, miR-614, miR-492, miR-10a, miR-622, miR-196b, miR-210, miR-939, miR-649, miR-801, miR-135b*, miR-148a, miR-194*, miR-21, miR-708, miR-222, miR-30a* and miR-323-3p, or
v) miR-122, miR-135b, miR-135b*, miR-136*, miR-186, miR-196b, miR-198, miR-203, miR-222, miR-23a, miR-34c-5p, miR-451, miR-490-3p, miR-492, miR-509-5p, miR-571, miR-614, miR-622 and miR-939,
   wherein said device is used for characterising a sample.

In one embodiment, said device comprises or consists of at least one probe or probe set for a miRNA selected from the group consisting of miR-411 and miR-198. In one embodiment, said device comprises or consists of at least one probe or probe set for miR-411 and at least one probe or probe set for miR-198.

In another embodiment, said device comprises or consists of at least one probe or probe set for a miRNA selected from the group consisting of miR-614 and miR-122. In one embodiment, said device comprises or consists of at least one probe or probe set for miR-614 and at least one probe or probe set for miR-122.

In yet another embodiment, said device comprises or consists of at least one probe or probe set for a miRNA selected from the group consisting of miR-614 and miR-93*. In one embodiment, said device comprises or consists of at least one probe or probe set for miR-614 and at least one probe or probe set for miR-93*.

In another embodiment, said device comprises or consists of at least one probe or probe set for a miRNA selected from the group consisting of miR-198, miR-34c-5p, miR-614, miR-492, miR-10a, miR-622, miR-196b, miR-210, miR-939, miR-649, miR-801, miR-135b*, miR-148a, miR-194*, miR-21, miR-708, miR-222, miR-30a* and miR-323-3p.

In yet another embodiment, said device comprises or consists of at least one probe or probe set for a miRNA selected from the group consisting of miR-122, miR-135b, miR-135b*, miR-136*, miR-186, miR-196b, miR-198, miR-203, miR-222, miR-23a, miR-34c-5p, miR-451, miR-490-3p, miR-492, miR-509-5p, miR-571, miR-614, miR-622 and miR-939.

In one embodiment, the device according to the present invention further comprises one or more probes or probe sets for a miRNA selected from the group consisting of hsa-miR-93, hsa-miR-93*, hsa-miR-411, hsa-miR-198, hsa-miR-34c-5p, hsa-miR-21, hsa-miR-708, hsa-miR-614, hsa-miR-196b, hsa-miR-939, hsa-miR-148a, hsa-miR-801, hsa-miR-886-5p, hsa-miR-210, hsa-miR-190b, hsa-miR-142-3p, hsa-miR-130b*, hsa-miR-649, hsa-miR-30a*, hsa-miR-650, hsa-miR-492, hsa-miR-922, hsa-miR-31, hsa-miR-219-1-3p, hsa-miR-432*, hsa-miR-130b, hsa-miR-100*, hsa-miR-222*, hsa-miR-222, hsa-miR-375, hsa-miR-135b*, hsa-miR-592, hsa-miR-494, hsa-miR-148a*, hsa-miR-635, hsa-miR-598, hsa-miR-622, hsa-miR-877, hsa-miR-875-5p, hsa-miR-451, hsa-miR-891a, hsa-miR-509-5p, hsa-miR-518d-3p, hsa-miR-648, hsa-miR-449b, hsa-miR-141*, hsa-miR-643, hsa-miR-575, hsa-miR-193b*, hsa-miR-217, hsa-miR-154*, hsa-miR-34b*, hsa-miR-7-2*, hsa-miR-147b, hsa-miR-584, hsa-miR-449a, hsa-miR-411*, has-miR-411, hsa-miR-589*, hsa-miR-216b, hsa-miR-379*, hsa-miR-216a, hsa-miR-219-5p, hsa-miR-486-3p, hsa-miR-153, hsa-miR-143*, hsa-miR-542-5p, hsa-miR-644, hsa-miR-944, hsa-miR-129-5p, hsa-miR-19a*, hsa-miR-377*, hsa-miR-640, hsa-miR-383, hsa-miR-208, hsa-miR-566, hsa-miR-200c*, hsa-miR-147, hsa-miR-374a*, hsa-miR-92b*, hsa-miR-888, hsa-miR-205, hsa-miR-129-3p, hsa-miR-499-5p, hsa-miR-194*, hsa-miR-543, hsa-miR-554, hsa-miR-141*, hsa-miR-766, hsa-miR-516a-3p, hsa-miR-215, hsa-miR-135b, hsa-miR-203, hsa-miR-194, hsa-miR-192, hsa-miR-133a, hsa-miR-133b, hsa-miR-654-5p, hsa-miR-154, hsa-miR-122, hsa-miR-125b-2*, hsa-miR-490-3p, hsa-miR-552, hsa-miR-187, hsa-miR-518f, hsa-miR-450b-5p, hsa-miR-656, hsa-miR-10a, hsa-miR-337-3p, hsa-miR-520c-3p, hsa-miR-493*, hsa-miR-512-3p, hsa-miR-374a, hsa-miR-30e*, hsa-miR-937, hsa-miR-376b, hsa-miR-639, hsa-miR-497*, hsa-miR-518e, hsa-miR-143, hsa-miR-323-3p, hsa-miR-335, hsa-miR-30c, hsa-miR-548b-5p, hsa-miR-590-5p, hsa-miR-548d-5p, hsa-miR-551b*, hsa-miR-487b, hsa-miR-33a*, hsa-miR-616*, hsa-miR-889, hsa-miR-628-3p, hsa-miR-455-3p, hsa-miR-184, hsa-miR-672, hsa-miR-373, hsa-miR-582-3p, hsa-miR-124, hsa-let-7a*, hsa-miR-551b, hsa-miR-513-3p and hsa-miR-330-5p.

In one embodiment, the device may be used for distinguishing between pancreas cancer (PAC and/or AAC) and normal pancreas; and/or distinguishing between pancreatic carcinoma (PAC and/or AAC) and chronic pancreatitis; and/or distinguishing between the combined class of pancreatic carcinoma (PAC) and ampullary adenocarcinoma from the combined class of normal pancreas and chronic pancreatitis.

In one embodiment, said device may be used with the miRNA classifier according to the present invention to classify a sample into either of the combined class of pancreatic carcinoma and ampullary adenocarcinoma and the combined class of normal pancreas and chronic pancreatitis.

In one embodiment, said device may be used with the miRNA biomarkers according to the present invention to determine if a sample belongs to either of the classes of pancreas cancer and normal pancreas; either of the classes of pancreas cancer and chronic pancreatitis; or either of the combined classes of pancreatic carcinoma and ampullary adenocarcinoma, and normal pancreas and chronic pancreatitis.

In one embodiment said device comprises between 1 to 2 probes or probe sets per miRNA to be measured, such as 2 to 3 probes, for example 3 to 4 probes, such as 4 to 5 probes, for example 5 to 6 probes, such as 6 to 7 probes, for example 7 to 8 probes, such as 8 to 9 probes, for example 9 to 10 probes, such as 10 to 15 probes, for example 15 to 20 probes, such as 20 to 25 probes, for example 25 to 30 probes, such as 30 to 40 probes, for example 40 to 50 probes, such as 50 to 60 probes, for example 60 to 70 probes, such as 70 to 80 probes, for example 80 to 90 probes, such as 90 to 100 probes or probe sets per miRNA of the present invention to be measured.

In another embodiment, said device has of a total of 1 probe or probe set for at least one miRNA to be measured, such as 2 probes, for example 3 probes, such as 4 probes, for example 5 probes, such as 6 probes, for example 7 probes, such as 8 probes, for example 9 probes, such as 10 probes, for example 11 probes, such as 12 probes, for example 13 probes, such as 14 probes, for example 15 probes, such as 16 probes, for example 17 probes, such as 18 probes, for example 19 probes, such as 20 probes, for example 21 probes, such as 22 probes, for example 23 probes, such as 24 probes, for example 25 probes, such as 26 probes, for example 27 probes, such as 28 probes, for example 29 probes, such as 30 probes, for example 31 probes, such as 32 probes, for example 33 probes, such as 34 probes, for example 35 probes, such as 36 probes, for example 37 probes, such as 38 probes, for example 39 probes, such as 40 probes, for example 41 probes, such as 42 probes, for example 43 probes, such as 44 probes, for example 45 probes, such as 46 probes, for example 47 probes, such as 48 probes, for example 49 probes, such as 50 probes or probe sets for at least one miRNA of the present invention to be measured.

It follows, that there may be one probe specific to a miRNA to be measured, or more than one probe specific to a miRNA to be measured—which may be called a probe set. In one embodiment, the device comprises 1 probe per miRNA to be measured, in another embodiment, said device comprises 2 probes, such as 3 probes, for example 4 probes, such as 5 probes, for example 6 probes, such as 7 probes, for example 8 probes, such as 9 probes, for example 10 probes, such as 11 probes, for example 12 probes, such as 13 probes, for example 14 probes, such as 15 probes per miRNA to be measured or analysed.

In one embodiment, the device may be a microarray chip; a QPCR Micro Fluidic Card; or may comprise QPCR tubes, QPCR tubes in a strip or a QPCR plate, comprising one or more probes for at least one miRNA and identified herein; selected from the group of i) miR-411 and miR-198, or
ii) miR-614 and miR-122, or
iii) miR-614 and miR-93*, or
iv) miR-198, miR-34c-5p, miR-614, miR-492, miR-10a, miR-622, miR-196b, miR-210, miR-939, miR-649, miR-801, miR-135b*, miR-148a, miR-194*, miR-21, miR-708, miR-222, miR-30a* and miR-323-3p, or
v) miR-122, miR-135b, miR-135b*, miR-136*, miR-186, miR-196b, miR-198, miR-203, miR-222, miR-23a, miR-34c-5p, miR-451, miR-490-3p, miR-492, miR-509-5p, miR-571, miR-614, miR-622 and miR-939.

In one embodiment, said device further comprises one or more probes for a miRNA selected from the group of hsa-miR-93, hsa-miR-93*, hsa-miR-411, hsa-miR-198, hsa-miR-34c-5p, hsa-miR-21, hsa-miR-708, hsa-miR-614, hsa-miR-196b, hsa-miR-939, hsa-miR-148a, hsa-miR-801, hsa-miR-886-5p, hsa-miR-210, hsa-miR-190b, hsa-miR-142-3p, hsa-miR-130b*, hsa-miR-649, hsa-miR-30a*, hsa-miR-650, hsa-miR-492, hsa-miR-922, hsa-miR-31, hsa-miR-219-1-3p, hsa-miR-432*, hsa-miR-130b, hsa-miR-100*, hsa-miR-222*, hsa-miR-222, hsa-miR-375, hsa-miR-135b*, hsa-miR-592, hsa-miR-494, hsa-miR-148a*, hsa-miR-635, hsa-miR-598, hsa-miR-622, hsa-miR-877, hsa-miR-875-5p, hsa-miR-451, hsa-miR-891a, hsa-miR-509-5p, hsa-miR-518d-3p, hsa-miR-648, hsa-miR-449b, hsa-miR-141*, hsa-miR-643, hsa-miR-575, hsa-miR-193b*, hsa-miR-217, hsa-miR-154*, hsa-miR-34b*, hsa-miR-7-2*, hsa-miR-147b, hsa-miR-584, hsa-miR-449a, hsa-miR-411*, hsa-miR-411, hsa-miR-589*, hsa-miR-216b, hsa-miR-379*, hsa-miR-216a, hsa-miR-219-5p, hsa-miR-486-3p, hsa-miR-153, hsa-miR-143*, hsa-miR-542-5p, hsa-miR-644, hsa-miR-944, hsa-miR-129-5p, hsa-miR-19a*, hsa-miR-377*, hsa-miR-640, hsa-miR-383, hsa-miR-208, hsa-miR-566, hsa-miR-200c*, hsa-miR-147, hsa-miR-374a*, hsa-miR-92b*, hsa-miR-888, hsa-miR-205, hsa-miR-129-3p, hsa-miR-499-5p, hsa-miR-194*, hsa-miR-543, hsa-miR-554, hsa-miR-141*, hsa-miR-766, hsa-miR-516a-3p, hsa-miR-215, hsa-miR-135b, hsa-miR-203, hsa-miR-194, hsa-miR-192, hsa-miR-133a, hsa-miR-133b, hsa-miR-654-5p, hsa-miR-154, hsa-miR-122, hsa-miR-125b-2*, hsa-miR-490-3p, hsa-miR-552, hsa-miR-187, hsa-miR-518f, hsa-miR-450b-5p, hsa-miR-656, hsa-miR-10a, hsa-miR-337-3p, hsa-miR-520c-3p, hsa-miR-493*, hsa-miR-512-3p, hsa-miR-374a, hsa-miR-30e*, hsa-miR-937, hsa-miR-376b, hsa-miR-639, hsa-miR-497*, hsa-miR-518e, hsa-miR-143, hsa-miR-323-3p, hsa-miR-335, hsa-miR-30c, hsa-miR-548b-5p, hsa-miR-590-5p, hsa-miR-548d-5p, hsa-miR-551b*, hsa-miR-487b, hsa-miR-33a*, hsa-miR-616*, hsa-miR-889, hsa-miR-628-3p, hsa-miR-455-3p, hsa-miR-184, hsa-miR-672, hsa-miR-373, hsa-miR-582-3p, hsa-miR-124, hsa-let-7a*, hsa-miR-551b, hsa-miR-513-3p and hsa-miR-330-5p.

The probes may be comprised on a solid support, on at least one bead, or in a liquid reagent comprised in a tube.

### Computer Program Product

It is a further aspect of the invention to provide a computer program product having a computer readable medium, said computer program product comprising means for carrying out any of the herein listed miRNA classifiers, models and methods.

It is a further aspect of the invention to provide a system comprising means for carrying out any of the herein listed methods.

It is an aspect of the present invention to provide a system for determining the presence of pancreatic carcinoma in an individual, said system comprising means for analysing the expression level of at least one miRNA in a sample obtained from an individual, wherein the expression level of said miRNAs is associated with pancreatic carcinoma, wherein said at least one miRNA is selected from the group consisting of
i) miR-411 and miR-198, or
ii) miR-614 and miR-122, or
iii) miR-614 and miR-93*, or
iv) miR-198, miR-34c-5p, miR-614, miR-492, miR-10a, miR-622, miR-196b, miR-210, miR-939, miR-649, miR-801, miR-135b*, miR-148a, miR-194*, miR-21, miR-708, miR-222, miR-30a* and miR-323-3p, or
v) miR-122, miR-135b, miR-135b*, miR-136*, miR-186, miR-196b, miR-198, miR-203, miR-222, miR-23a, miR-34c-5p, miR-451, miR-490-3p, miR-492, miR-509-5p, miR-571, miR-614, miR-622 and miR-939.

In another aspect, the present invention provides a system for performing a diagnosis on an individual, comprising:
i) means for analysing the miRNA expression profile of a sample obtained from said individual, and
ii) means for determining if said individual has a condition selected from pancreatic cancer, pancreatic adenocarcinoma, ampullary adenocarcinoma and chronic pancreatitis,
wherein said miRNA expression profile comprises at least one miRNA selected from the group consisting of
i) miR-411 and miR-198, or
ii) miR-614 and miR-122, or
iii) miR-614 and miR-93*, or
iv) miR-198, miR-34c-5p, miR-614, miR-492, miR-10a, miR-622, miR-196b, miR-210, miR-939, miR-649, miR-801, miR-135b*, miR-148a, miR-194*, miR-21, miR-708, miR-222, miR-30a* and miR-323-3p, or
v) miR-122, miR-135b, miR-135b*, miR-136*, miR-186, miR-196b, miR-198, miR-203, miR-222, miR-23a, miR-34c-5p, miR-451, miR-490-3p, miR-492, miR-509-5p, miR-571, miR-614, miR-622 and miR-939.

In another aspect, the present invention provides a computer program product having a computer readable medium, said computer program product providing a system for predicting the diagnosis of an individual, said computer program product comprising means for carrying out any of the steps of any of the methods as disclosed herein.

In another aspect, the present invention provides a system as disclosed herein wherein the data is stored, such as stored in at least one database.

### Kit-of-Parts

It is also an aspect to provide a kit-of-parts comprising the device according to the present invention, and at least one additional component.

In one embodiment, the additional component may be used simultaneously, sequentially or separately with the device.

In one embodiment, said additional component comprises means for extracting RNA such as miRNA from a sample; reagents for performing microarray analysis and/or reagents for performing QPCR analysis.

In another embodiment, said kit may comprise instructions for use of the device and/or the additional components.

In a further embodiment, said kit comprises a computer program product having a computer readable medium as detailed herein elsewhere.

Sequences

| miR name | Sequence |
|---|---|
| hsa-miR-10a | uacccuguagauccgaauuugug |
| hsa-miR-21 | uagcuuaucagacugauguuga |
| hsa-miR-30a* | cuuucagucggauguuugcagc |
| hsa-miR-34c-5p | aggcaguguaguuagcugauugc |
| hsa-miR-93 | caaagugcuguucgugcagguag |
| hsa-mir-122 | uggagugugacaaugguguuug |
| hsa-miR-135b* | auguagggcuaaaagccauggg |
| hsa-miR-148a | ucagugcacuacagaacuuugu |
| hsa-miR-194* | ccaguggggcugcuguuaucug |
| hsa-miR-196b | uagguaguuuccuguuguuggg |
| hsa-mir-198 | gguccagaggggagauagguuc |
| hsa-miR-210 | cugugcgugugacagcggcuga |
| hsa-miR-222 | agcuacaucuggcuacugggu |
| hsa-miR-323-3p | cacauuacacggucgaccucu |
| hsa-mir-411 | uaguagaccguauagcguacg |
| hsa-miR-492 | aggaccugcgggacaagauucuu |
| hsa-mir-614 | gaacgccuguucuugccaggugg |
| hsa-miR-622 | acagucugcugagguuggagc |
| hsa-miR-649 | aaaccuguguuguucaagaguc |
| hsa-miR-708 | aaggagcuuacaaucuagcuggg |
| hsa-miR-801 | dead miRNA entry |
| hsa-miR-939 | uggggagcugaggcucugggggug |
| hsa-miR-93* | acugcugagcuagcacuucccg |
| hsa-miR-122 | uggagugugacaaugguguuug |
| hsa-miR-135b | uauggcuuuucauuccuauguga |
| hsa-miR-136* | caucaucgucucaaaugagucu |
| hsa-miR-186 | caaagaauucuccuuuugggcu |
| hsa-miR-203 | gugaaauguuuaggaccacuag |
| hsa-miR-23a | aucacauugccagggauuucc |
| hsa-miR-451 | aaaccguuaccauuacugaguu |
| hsa-miR-490-3p | caaccuggaggacuccaugcug |
| hsa-miR-509-5p | uacugcagacaguggcaauca |
| hsa-miR-571 | ugaguuggccaucugagugag |

### EXAMPLES

MicroRNA Expression Profiles Associated with Pancreatic Cancer

### Abstract

### Purpose:

1) Define the global microRNA (miR) expression pattern in pancreatic cancer (PC), normal pancreas (NP) and chronic pancreatitis (CP); 2) Validate reported diagnostic miR profiles for PC; and 3) Discover new diagnostic miRs and combinations of miRs in PC tissue without micro-dissection.

### Experimental Design:

MiR expression patterns in formalin fixed paraffin embedded tissue blocks from 277 pancreatic adenocarcinomas and ampullary adenocarcinomas (A-AC) were analyzed using a miR low density assay (664 human miRs) and compared to CP and NP.

### Results:

Eighty-three miRs were differently expressed between PC and NP (42 had higher and 41 reduced expression in PC). Thirty-two miRs were differently expressed between PC and CP (17 higher and 15 reduced). MiR-614, miR-492, miR-622, miR-135b* and miR-196 were most differently expressed. MiR-143/143*, miR-148a, miR-205 and miR-375 were validated. The miR signatures of PC and A-AC were highly correlated (correlation=0.99). The earlier reported diagnostic miR profile for PC, the difference between mirR-196b and miR-217, was validated. A more significant diagnostic profile, difference between miR-411 and miR-198 (P=2.06E-54), and a classifier using 19 miRs (accuracy 97%) were identified.

### Conclusions:

Systematic differences were found in miR expressions between PC tissue including desmoplasia compared to tissue from CP and NP. A 19 miRs classifier was constructed, which discriminates PC and A-AC from CP and NP with an accuracy of 97%. We validated that combinations of just two previously identified microRNAs can separate neoplastic from non-neoplastic samples. Prospective studies are needed to evaluate if our panel of miRs is useful for early diagnosis of patients with PC.

### Translational Relevance

The regulatory function and stable characteristic of microRNAs make them promising new biomarkers. MicroRNA can be used to get an understanding of cancer genetics and protein synthesis in cancer. But microRNA can also be used as independent biomarkers in prognostic profiles or profilling of different tissues. Recent studies have shown a distinct microRNA expression pattern in pancreatic cancer tissue that differentiates it from normal pancreas and chronic pancreatitis. Consensus and reproducibility among the studies of microRNAs performed on different microarray or quantitative-RT-PCR platforms is necessary before miRs can be implemented clinical practise. The present study, where microRNAs are used as independent biomarkers to separate pancreatic cancer tissue from normal pancreas and chronic pancreatis, validate the microRNA expression pattern from other studies. Several newly discovered microRNAs are included in the pancreatic cancer profile. And new ways of combining the growing library of human microRNAs strengthen the ability to identify cancer samples.

### Introduction

Pancreatic cancer (PC) is the 4^{th} most common cause of cancer death in United States and Europe. The prognosis of patients with pancreatic cancer is dismal with a 5-year survival rate of less than 5% [1-3]. Most pancreatic cancers are ductal adenocarcinomas (PDAC). Early diagnosis of PC is difficult. Most patients therefore have locally advanced or metastatic pancreatic cancer at time of diagnosis [3]. Less than 20% of the patients can be operated with curative intent and with a 5-years survival after surgery below 20% [4]. The clinical and histological similarity between PC and chronic pancreatitis adds another dimension to the diagnostic challenge. Thus, novel strategies for early diagnosis of patients with pancreatic cancer are urgently needed. Seven to twelve percent of all periampullary carcinomas are adenocarcinomas of the Ampulla of Vater (ampullary adenocarcinomas; A-AC) [5-7]. The prognosis is better with a 5-years survival after surgery of 40% [6, 7]. One of the reasons is that even small A-AC cause jaundice so more patients are operated at an early tumour stage and without lymph node metastasis. Furthermore, biological differences between PC and A-AC exist.

MicroRNAs (miRs) are 19-25-nucleotide-long non-coding RNAs which after cleavage into their mature form bind to the RNA-induced silencing complex (RISC) and regulate gene expression posttranscriptionally by a binding of specific mRNA. They have provided important impact in the understanding of cancer biology. MiRs regulate many genes known to play important roles in oncogenesis, angiogenesis and tissue differentiation supporting their involvement in cancer development and progression [8-13]. More than 1048 human miR sequences have been discovered to date, and the number is still increasing (http://www.mirbase.org/index.shtml, last accessed Sep. 12, 2010). MiRs have highly tissue-specific expression patterns [14-17] and are, therefore, interesting new biomarkers with a potential for earlier diagnosis of pancreatic cancer. It has been demonstrated that PC tissue have a miR expression pattern (e.g. miR-15b, miR-21, miR-95, miR-103, miR-107, miR-148a, miR-155, miR-196a, miR-200, miR-210, miR-217, miR-221, miR-222, miR-375) that differs from tissue of normal pancreas and chronic pancreatitis [15, 18-24]. Results presented by Bloomston et al. [18] and Szafranska et al. [24] gave promises of significant clinical impact of miR expression profiles to separate tissue from PC from normal pancreas and chronic pancreatitis. The results from Szafranska et al. included a diagnostic combination of two miRs (miR-196a and miR-217) which has been commercialized by ASURAGEN [24]. MiRs are stable in formalin fixed paraffin embedded (FFPE) samples, and in most of the published studies microdissection has been used to isolate PC cells in the FFPE tumour blocks. PC cells are very often located in small groups surrounded by an abundant stromal tissue [25]. Important information related to miRs from the stromal tissue can therefore be lost if microdissection of the cancer cells is used. Furthermore, miR studies of PC and A-AC tissue samples without microdissection are more similar to daily clinical practise where a needle or fine needle aspiration biopsy is collected from the tumour or a metastasis.

The aim of the present study was to validate the results of diagnostic miR profiles for pancreatic cancer without microdissection of the tumour samples. We conducted a large miR expression study in 328 subjects operated for disease in the pancreas or surrounding peri-ampullary tissue including 170 patients with PC and 107 with A-AC using genome-wide miR profiling. Their miR expression profiles were compared with the profile in 23 subjects with chronic pancreatis and 28 controls without pancreatic diseases.

### Materials and Methods

### Pancreas Samples

### Patients:

From a database with 328 consecutive patients operated for lesions in the pancreas at Herlev Hospital, Copenhagen University Hospital, between December 1976 and June 2008 the 277 patients operated with radical intentions for pancreatic adenocarcinomas (mostly of ductal origin) (n=170) and A-AC (n=107) were included in the present study. The clinical information was updated Mar. 22, 2010. 257 patients underwent a pancreaticoduodenectomy (Whipple procedure), 13 a distal pancreatectomy, and 7 a total pancreatectomy. The characteristics of the patients are shown below:

| | Pancreatic cancer | Ampullary Adenocarc. |
|---|---|---|
| Characteristic | n = 170 | n = 107 |
| Age, years median (range) | 63 (33-85) | 64 (31-79) |
| Sex, male/female | 88/82 | 45/62 |
| | 52%/48% | 42%/58% |
| T stage, 1/2/3 | 15/29/126 | 4/41/62 |
| | 9%/17%/74% | 4%/38%/58% |
| Lymph nodes, 0/1/>1 | 66/39/65 | 67/20/20 |
| | 39%/23%/38% | 63%/19%/19% |
| Stage, IA/IB/IIA/IIB | 9/14/43/104 | 3/29/34/41 |
| | 5%/8%/25%/61% | 3%/27%/32%/38% |
| Histological grade | 4/75/42/48 | 12/27/30/38 |
| undifferentiated/poor/ | 2%/44%/25%/28% | 11%/25%/28%/36% |
| moderate/well | | |

Clinical characteristics of the patients with pancreatic cancer and ampullary adenocarcinomas

### Controls:

Archival FFPE tissue blocks from patients with chronic pancreatitis (n=23) and normal pancreas (n=28) were collected from the Departments of Pathology at Herlev Hospital, Copenhagen University Hospital (chronic pancreatitis n=3, normal pancreas n=4), Departments of Pathology at Haukeland University Hospital, Bergen (normal pancreas n=4), and from Department of General, Visceral and Transplant Surgery, University of Heidelberg (chronic pancreatic n=20, normal pancreas n=20). Normal pancreas samples are taken from donor patients and patient with traumatic lesions in tissue around the pancreas which led to removal of healthy pancreas.

The study was approved by the local Ethical Committee (protocol H-KA-20060181) in Region Hovedstaden, Denmark, the Ethical Committee in Bergen, Norway and the Ethical Committee in Heidelberg, Germany. The Danish Registry of Human Tissue Utilization was consulted.

### Pathology:

New sections from the FFPE tissue blocks, representing tumour and normal pancreas, from each patient and controls were stained with hematoxylin and eosin (HE) and examined by two experienced pathologist (AR, TH). All PC and A-AC were classified and graded according to WHO criteria (30). Tissue blocks representing cancer tissue, chronic pancreatitis and normal pancreas respectively were selected for miR analysis.

### MiR Analysis and Quality Control Procedures

Tumour blocks and tissue blocks from controls with normal pancreas and chronic pancreatitis were treated the same way. Three 10 μm sections were cut from each of the FFPE samples for RNA extraction and placed in a sterile eppendorf tube. Small RNA was extracted from FFPE tissue using High Pure miRNA Isolation Kit (Roche) according to the manufactures' instructions. In brief, the tissue sections were deparaffinized in xylene and ethanol, then treated with proteinase K and finally RNA was isolated using the one-column spin column protocol for total RNA. The Concentration of RNA was assessed by absorbance spectrometry on NanoDrop X-1000 (Thermo Fisher Scientific, Inc.). The miRNA profiling was performed on TaqMan® Array Human MicroRNA A+B Cards v2.0 (Applied Biosystems) using the manufactures reagents and instructions. Each array analyzes 664 different human miRs and enables a comprehensive expression profile consistent with Sanger miRBase v14 (human). Briefly, the RNA was transcribed into cDNA in two multiplex reactions each containing 200 ng of RNA and either Megaplex RT Primer A Pool or Pool B pool and using the TaqMan MicroRNA Reverse Transcription Kit in a total volume of 14 μl. Prior to loading the 12 cycle preamplification reaction was performed using 2.5 μl cDNA in a 25 μl reaction. Each of the arrays was loaded with 800 μl Universal PCR MasterMix assay containing 1/40 of the preamplification reaction and run on the 7900HT Fast Real-Time PCR System. All samples were analyzed at a certified centre, AROS Applied Biotechnology A/S, Aarhus, Denmark.

### Statistical Analysis

Raw Ct values where pre-processed in the following steps: 1) missing values and Ct values above 32 was flagged: 2) repeat measurements (excluding flagged values) where averaged; 3) features that were flagged in more than a given percent of samples were removed from the dataset; 4) missing values were set to Ct=40; and 5) quantile normalization was performed [26]. For QC of samples, the threshold in step 3 was set to 80%. Normalized data was inspected for outliers and potential technical bias from sample quality, sample purification date and TLDA array batch. No heavy technical bias was observed. However, 21 samples were identified as outliers. Most samples' Ct density curves were bimodal with peaks around 29 and 40. In some cases, the peak around 40 was relatively high compared to the peak around 29 and these samples corresponded well to outliers identified by principal component analysis. We therefore removed samples from the dataset if the ratio between the peaks at Ct>32 vs. Ct<32 was above 0.9 (outlier criteria 1:density ratio>0.9) or if their average correlation (Pearson correlation) with other samples in the dataset was below 0.70 (outlier criteria 2: average correlation<0.7). Furthermore, samples that were close to failing both criteria were also categorized as outliers (outlier criteria 3:density ratio>0.8 and average correlation<0.77). Samples that passed QC was pre-processed as described above with the threshold in step 3 now set to 95%. Analyses comparing ΔCt of two individual miRs between samples are based on un-normalized Ct values while the remaining analyses are based on normalized data. Hierarchical cluster analysis is based on ‘1-pearson correlation’ distances and ward linkage.

All two-class tests are based on Student's t-test assuming equal variance, and multiclass tests are based on F-tests assuming equal variance. All reported P-values are corrected for multiple comparisons (Bonferroni method).

For classification, we have fitted a regularized multinomial regression model using lasso [27]. The complexity of the fitted model is controlled by the penalty factor λ. The lower it is, the lower the penalty, resulting in more complex models. Thus, −log λ is used as a measure of model complexity. Classification performance was estimated by 10-fold cross validation repeated 10 times. For each 10-fold cross validation the dataset was split randomly into 10 equally sized test sets, while remaining samples were used for model fitting.

### Results

### RNA and Array Quality

RNA extraction was satisfying in all PC, A-AC, chronic pancreatitis and control samples (mean 260 nm/280 nm absorbance ratio was 1.85). All 328 samples (PC=170, A-AC n=107, chronic pancreatitis n=23, and normal pancreas n=28) were therefore considered for further analysis. Ten (6%) samples from PC and eleven (10%) samples from A-AC could be excluded according to the criteria described in “Materials and Methods”. The final dataset, consisting of 307 samples (PC n=160, A-AC n=96, chronic pancreatitis n=23, and normal pancreas n=28), was pre-processed as described in “Materials and Methods”, resulting in a pre-processed dataset comprising 307 samples and 475 miRs for further analysis. Of the remaining miRs, 342 were specifically expressed in at least one of the tissues.

Correlations Between miRs Differentiating Pancreatic Cancer from Controls

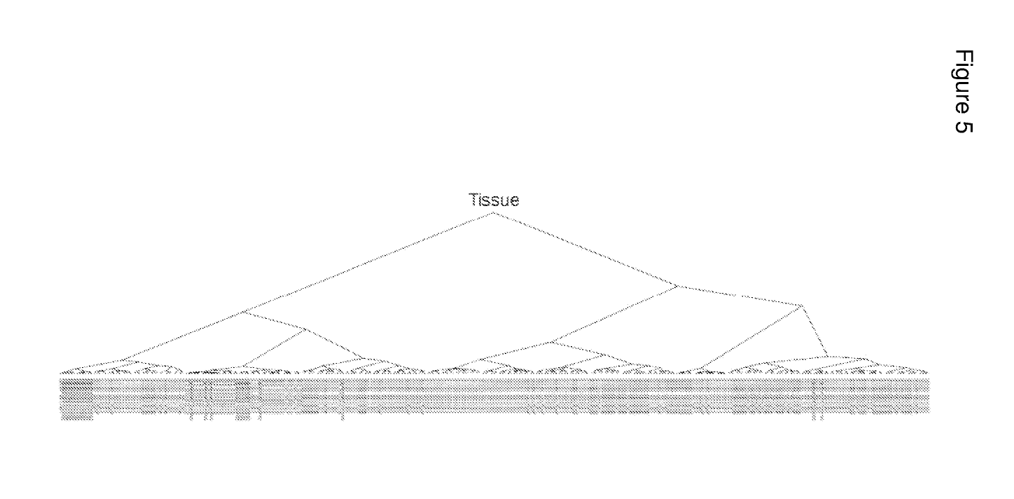

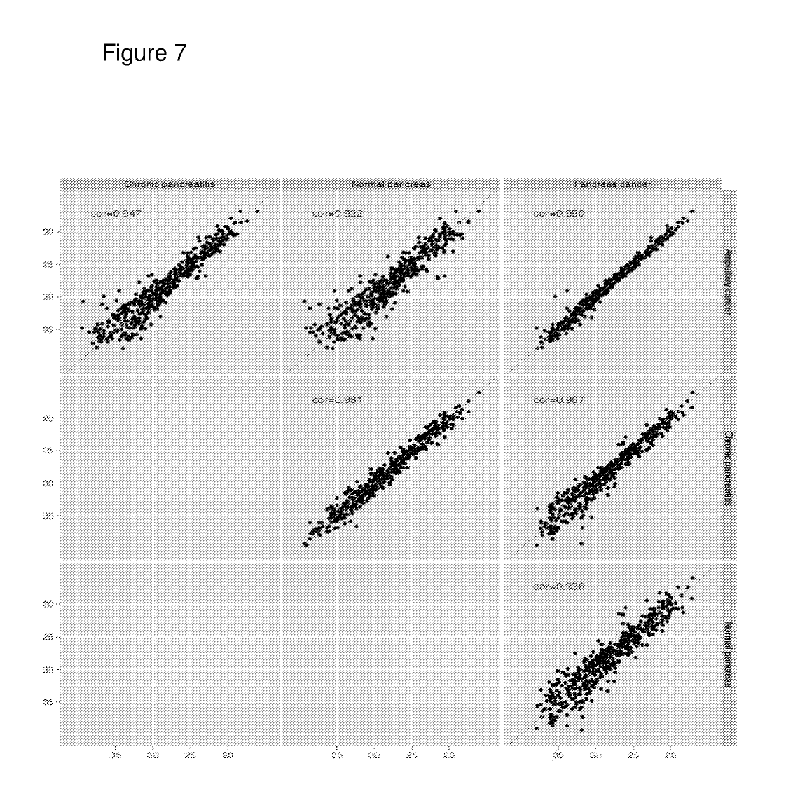

Cluster analyses of the samples from PC, A-AC, normal pancreas and chronic pancreatitis are illustrated in FIG. 5. Most normal pancreas and chronic pancreatitis samples clustered together. Scatter plots and correlations between the different groups are shown in FIG. 7. PC was most similar to A-AC (Pearson correlation=0.990), most different from normal pancreas (0.936), and showing intermediate difference from chronic pancreatitis (0.967). Chronic pancreatitis had a relatively high correlation to normal pancreas (0.981). A-AC and normal pancreas was least correlated (0.922).

### MicroRNA Expression Patterns in PC, A-AC and Chronic Pancreatitis Compared to Normal Pancreas Tissue

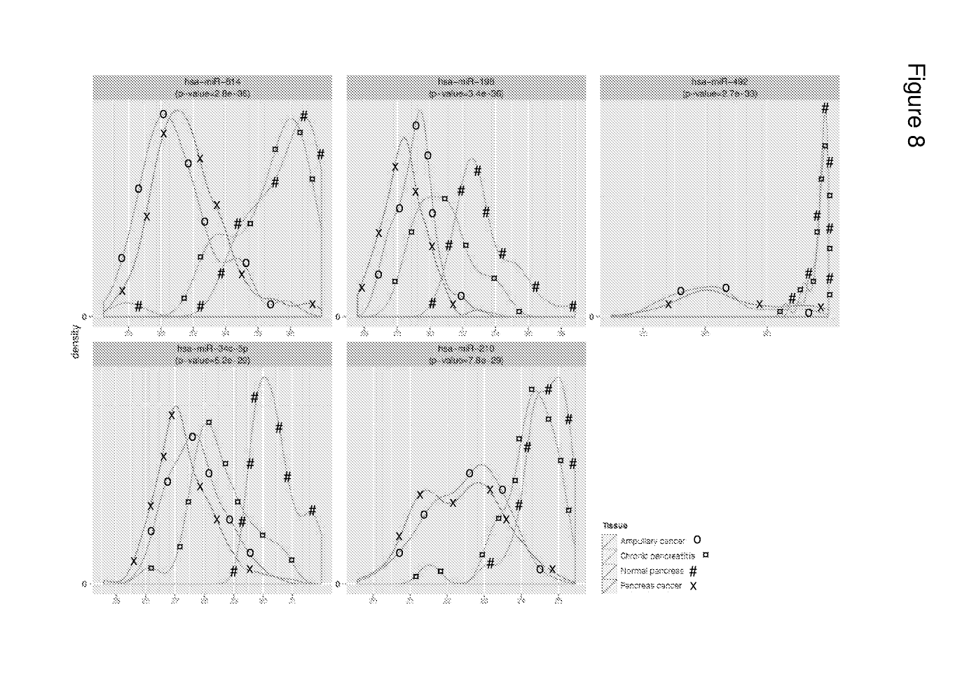

We compared miR profiles of PC, A-AC, chronic pancreatitis and normal pancreas using class comparison analysis. Five miRs were differentially expressed between PC and A-AC (miR-654-5p and miR-205 was expressed at higher levels in PC; miR194*, miR-187, and miR-552 were expressed at higher levels in A-AC). Eighty-four miRs were differentially expressed between PC and normal pancreas (43 miRs at higher levels in tumours; 41 miRs at lower levels in tumours) (P<0.05). One-hundred- and -ten miRs were differentially expressed between A-AC tumours and normal pancreas (55 miRs at higher levels in tumours; 55 miRs at lower levels in tumours) (P<0.05). FIG. 8 shows tissue comparison density plots for selected miRs. Table 4 shows all miRs significantly differentially expressed in PC, A-AC, chronic pancreatitis and normal pancreas.

PC Vs. Normal Pancreas:

The five most significantly differentially expressed miRs were miR-198, miR-34-c-5p, miR-21, miR-708 and miR-614) (Table 1). The most up-regulated (based on fold change) miRs in PC were miR-614, miR-198, and miR-196b. The most down-regulated miRs in PC were miR-216b, miR-217, and miR-148a* (Table 1). Nine of the significantly differentially expressed miRs described by Bloomston et al. [18] were also found in our study (miR-21, miR-100*, miR-143/miR-143*, miR-148a, miR-205, miR-210, miR-222, and miR-375). Eleven of the significantly differentially expressed miRs described by Szafranska et al. [19, 24] were also found in our study with very similar dm-values and fold change (miR-31, miR-130b, miR-143/miR-143*, 148a, miR-196b, miR-205, miR-210, miR-216, miR-217, miR-222/miR-222*, and miR-375) (Table 1).

A-AC Vs. Normal Pancreas:
The five most significantly differentially expressed miRs were miR-198, miR-10a, miR-650, miR-34-c-5p and miR-30a. The most up-regulated and down regulated miRs were miR-492, miR-143*, miR-614 and miR-216a/miR-216b, miR-891a, miR-217 respectively (Table 4).
PC and Chronic Pancreatitis Vs. Normal Pancreas:

MiR-198 and miR-650 had higher expression in both PC and chronic pancreatitis compared to normal pancreas tissue. MiR-130b, miR-141*, miR-194* and miR219-1-3p had reduced expression in both PC and chronic pancreatitis (Table 1).

### MicroRNA Expression Patterns in PC and A-AC Compared to Chronic Pancreatitis Tissue

In PC and A-AC, 32 and 56 miRs respectively were significantly differentially expressed compared to chronic pancreatitis.

PC vs. Chronic Pancreatitis:

The five most significantly differentially expressed miRs were miR-614, miR-492, miR-622, miR-135b* and miR-196b. The most up-regulated (compared on fold change) miRs were miR-492, miR-614, and miR-205. The most down-regulated miRs were miR-122, miR-891a, and miR-148a* (Table 1). MiR-148a was also found to be significantly down-regulated by Bloomston et al. [18]. Szafranska et al. found that miR-148a, miR-196b and miR-196a, miR-205 were differentially expressed in PC and chronic pancreatitis in their studies [19, 24] (Table 1).

A-AC Vs. Chronic Pancreatitis:

The differences in expression were very similar to the differences in PC. The five most significantly differentially expressed miRs were miR-492, miR-622, miR-614, miR-147b and miR-135b*. The most up-regulated and down-regulated miRs were miR-492, miR-194*, miR-614 and miR-891a, miR-129-3p, miR-122 respectively (Table 4).

A microRNA Classifier to Distinguish Pancreatic Cancer Samples from Normal Pancreas and Chronic Pancreatitis

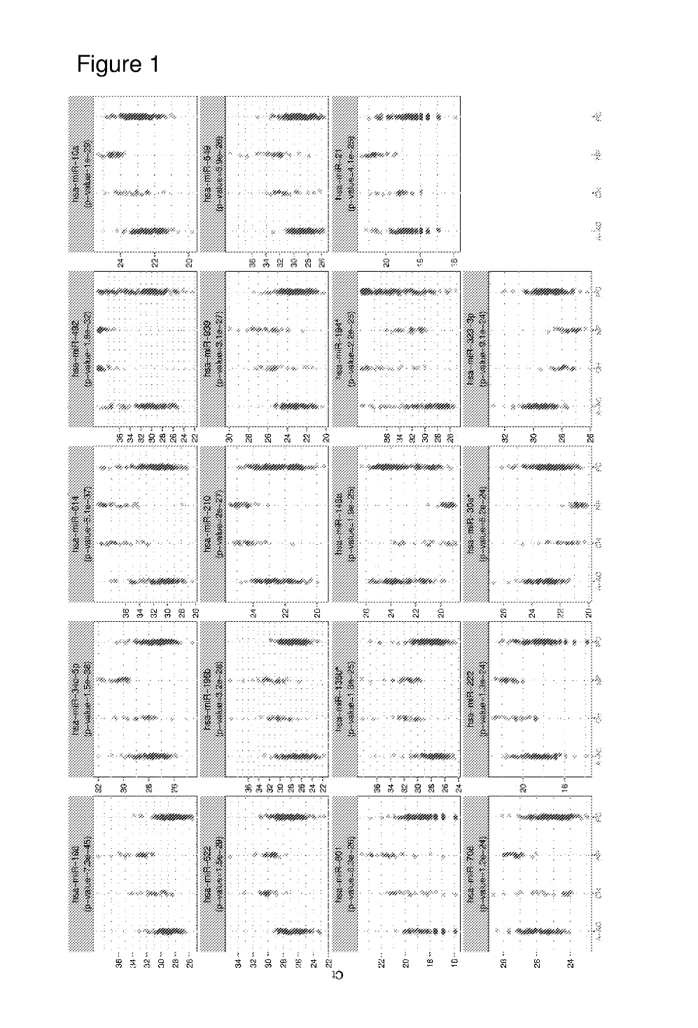

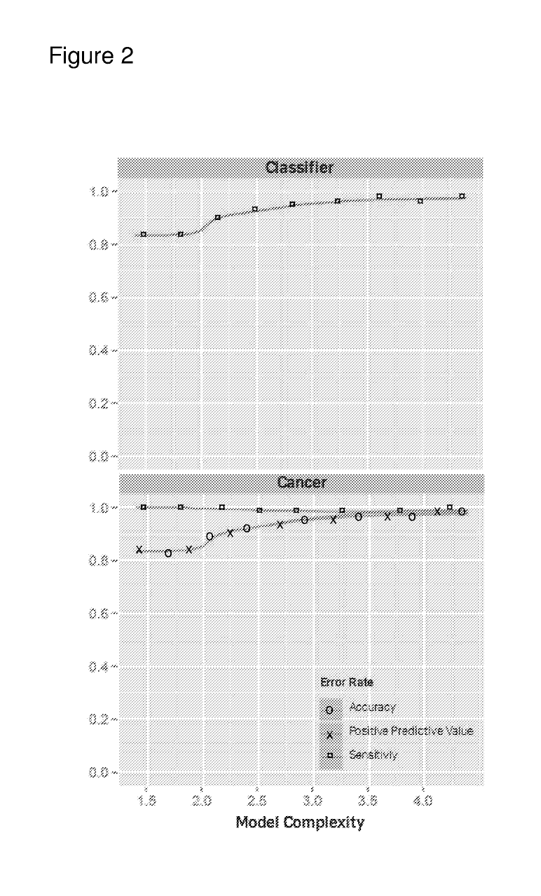

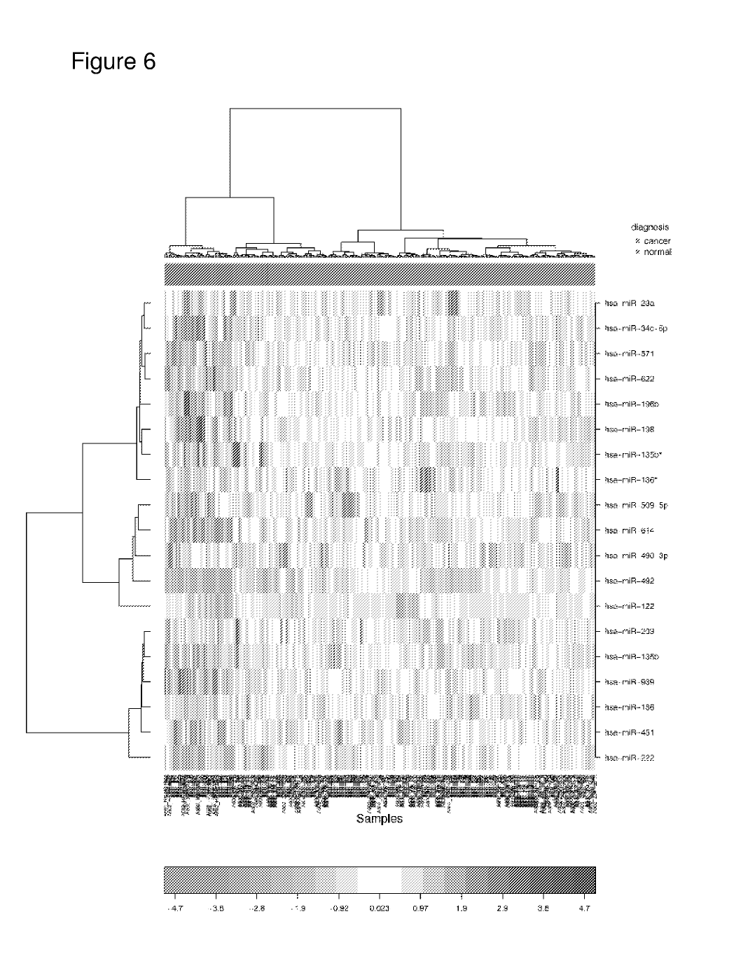

FIG. 1 shows strip charts of the nineteen (miR-198, miR-34c-5p, miR-614, miR-492, miR-10a, miR-622, miR-196b, miR-210, miR-939, miR-649, miR-801, miR-135b*, miR-148a, miR-194*, miR-21, miR-708, miR-222, miR-30a* and miR-323-3p) most significant miRs when comparing PC, A-AC, chronic pancreatitis and normal pancreas samples (F-test). FIG. 2 illustrates the lasso classifier performance (average+/−standard deviation for 10×10-fold cross-validation). An average prediction accuracy of 0.98 was obtained at a complexity of 4.2 corresponding to approximately 25 miRs on average (in each cross-validation classifier). When examining the classification performance graph it appeared slightly over-fitted at the high model complexities. Therefore, we estimate that a more robust classifier exists at a model complexity around 3.5 where the curve breaks close to 98% accuracy. When building a classifier on the full dataset with this model complexity, the classifier uses the 19 miRs listed in Table 2. This panel of miRs separates samples containing PC or A-AC cells from non-neoplastic tissue samples with a sensitivity of 0.985, a positive predictive value of 0.978 and an accuracy of 0.969. FIG. 6 shows a heat-map based on the 19 classifier miRs.

Simple Combinations of Two microRNAs

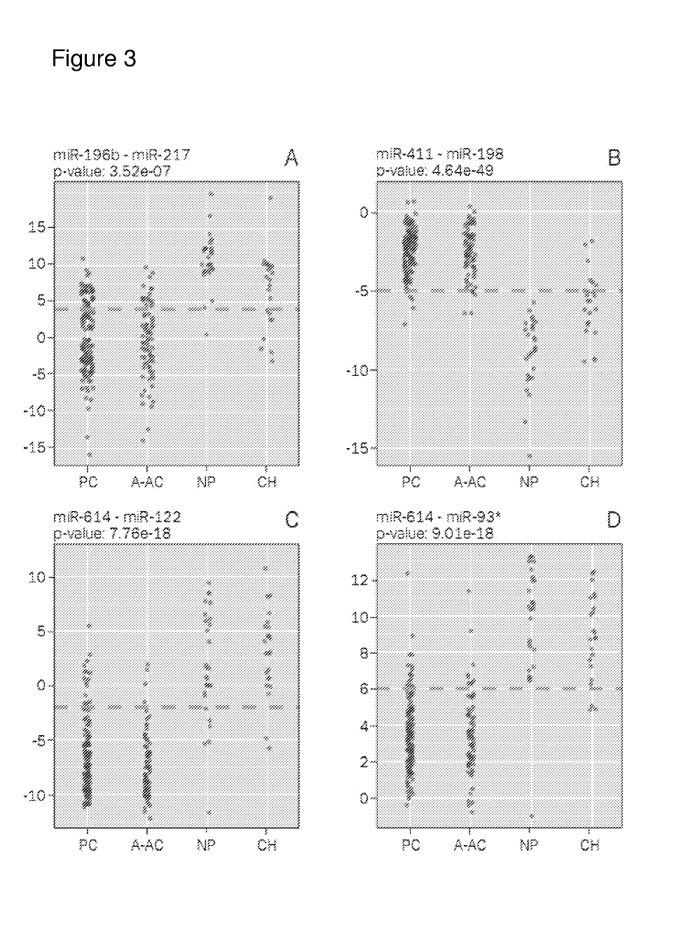

The difference in each patients expression of miR-196b and miR-217 (i.e. the AsuraGen test) in our setting is illustrated in FIG. 3A (the Applied Biosystem assay measures only miR-196b, one nucleotide different from miR-196a). These 2 miRs can separate PC from chronic pancreatitis (P=3.52e-7) and normal pancreas (P=8.59e-20). In our analysis, this combination of microRNAs performed best with more than 20% tumour cells in the samples. FIG. 3 B-D illustrate three other combinations of miRs (miR-411-miR-198; miR-614-miR-122; and miR-614-miR-93*) that perform better than the combination suggested by Szafranska et al and Asuragen. The overall best combination to separate PC and A-AC from normal pancreas and chronic pancreatitis was the difference between miR-411 and miR-198 (P=4.64e-49) (FIG. 3B). The difference in miR-614 and miR-122 was the best combination to separate PC from chronic pancreatitis (P=7.76e-18) (FIG. 3C). The best combination to separate PC from normal pancreas was the difference in miR-411 and miR-198 (P=5.17e-43). All results for the three combinations of miR-93*, miR-122, miR-198, miR-411 and miR-614 are listed in Table 3. FIG. 3A-D illustrate that the combinations of 2 miRs perform very well for 20% tumour cells in the sample.

### Discussion

This is one of few larger miR studies of patients operated for PC and A-AC. We used non micro-dissected tumour samples and a commercial miR microarray with the primary goals to validate the recently described diagnostic miR expression profile in patients with PC and to identify new diagnostic profiles of miRs for PC. Five miRs (up-regulated: miR-143/143*, miR-205 miR-210; down-regulated: miR-148a, miR-375) were significantly differentially expressed between PC and normal pancreas in the study by Bloomston et al. (14), in both studies by Szafranska et al. (15)[24] and in our study. In the same four studies miR-148a was down-regulated in PC compared to chronic pancreatitis. The usefulness of the AsuraGen test was confirmed in our study, i.e. the difference in expression of miR-196b and miR-217 could separate PC from chronic pancreatitis in samples with only 20% cancer cells. Interestingly, we also found that the differences in expression of miR-614 and miR-122 or miR-93* in pancreas tissue were even better to separate PC from chronic pancreatitis. The best combination to separate PC and A-AC from normal pancreas tissue and chronic pancreatitis was the difference in expression of miR-411 and miR-198 even if the amount of cancer cells was low in the sample. Furthermore, we identified a diagnostic panel of 19 miRs that separated samples containing PC or A-AC cells from non-neoplastic tissue samples with high sensitivity, predictive value and accuracy. These results were independent of the amount of tumour tissue. This is all novel observations. Fourteen (miR-34c-5p, miR-122, miR-135b, miR 135b*, miR-136*, miR-198, miR-451, miR-490-3p, miR-492, miR-509-5p, miR-571, miR-614, miR-622, miR-939) of the nineteen miRs in the classifier are not included in earlier reports of PC miR-profiles and nine of them are amongst the most stable miRs in our classifier (Table 2).

There was a high correlation between the miR expression in PC and A-AC tissue, and we found only 5 miRs with significant differential expression between these two types of pancreatic cancers. MiR-492, miR-614, miR-198, and miR-196b were expressed at higher level in both PC and A-AC compared to normal pancreas and chronic pancreatitis, and these miRs were among the most stable in our 19 miR diagnostic test.

MiR-492 and miR-614 have not been described in pancreatic cancer tissue or fibrotic tissue, and no functional studies are reported for these miRs in tumour development. But miR-492 is highly expressed in retinoblastoma [28]. Patients with colorectal cancer and a miR-492 C/G or G/C genotype had significantly shorter progression free survival than patients with miR-492 C/C genotype [29].

MiR-122 and miR-93* were included in our panel of useful miRs to discriminate PC from chronic pancreatitis, and miR-122 expression was significantly decreased in PC. MiR-122 and MiR-93* expression have not been described in pancreatic cancer before, but miR-122 expression is lower in liver cancers with intra-hepatic metastases and it regulates tumourigenesis negatively [30]. Mir-93 is increased in liver tumourigenesis [31].

In accordance with others, we found that miR-148a, miR-216b and miR-217b were some of the miRs with the most significantly decreased expression in PC compared to normal pancreas tissue and chronic pancreatitis [19]. It has recently been reported that miR-217 was down regulated in 76% of PC tissue and in all PC cell lines tested when compared to normal pancreas tissue and normal pancreas cell lines, and over-expression of miR-217 in PC cells inhibited tumour cell growth in vivo and in vitro [32]. Furthermore, miR-217 expression was negatively related with KRAS protein expression, and up-regulation of miR-217 decreased KRAS protein level and reduced the constitutive phosphorylation of AKT in the downstream PI3K-AKT pathway involved in cell growth, differentiation, proliferation and survival [32]. Yu et al. [33] found that miR-96 suppresses KRAS and functions as a tumour-suppressor in pancreatic cancer cells. We did not find miR-96 significantly down-regulated in tissue from PC and A-AC compared to chronic pancreatitis and normal pancreas.

We have validated the down-regulation of miR-148a and miR-375 in PC. Down regulation of miR-148a is an early marker of PC and is already decreased in pre-neoplastic PanIN lesions. Hypermethylation of the encoding DNA region is responsible for the repression of miR-148a [34]. MiR-375 is an islet-cell specific regulator of insulin secretion [35, 36] and was in our study significantly decreased in PC and A-AC compared to normal pancreas tissue.

PC is a poorly vascularised cancer, and hypoxia and resistance to chemotherapy are key features [37]. MiR-210 over-expression is related to hypoxic microenvironment in cancer, where this miR is involved in DNA-repair and angiogenesis [37-39]. We found miR-210 over-expressed in both PC and A-AC samples.

Several miRs (miR-130b, miR-141*, miR-194*, miR-198, miR-219-1-3p, miR-650) were significantly differently expressed in both PC and chronic pancreatitis compared to normal pancreas. MiR-130b and miR-141* belong to the miR-200 family, which is down regulated in cells undergoing epithelial to mesenchymal transition (EMT). EMT facilitates tissue remodelling in embryonic development and is an essential early step when tumours metastasize [40, 41, 41]. The miR-200 family is regarded as a tissue specific group of miRs, highly expressed in the endocrine glands including the pancreas [42]. MiR-194* is suggested to play an important role in maturation of intestinal epithelial cells [43].

Pancreatic cancer is characterized by a prominent desmoplastic stroma [25, 44]. This phenomenon, termed stromal reaction, includes activation of fibroblasts and myofibroblasts transformation, inflammation, enhanced secretion of cytokines, matrix proteins and metalloproteinases, and neovasculation. The stroma plays essential aspects of tumour proliferation and progression, cell death, matrix remodelling and angiogenesis, and subsequently promotes tumour growth and progression of metastatic disease. In our study the stromal tissue could contribute to a distinct miR profile for PC, since we analyzed non-micro-dissected tumour samples. The observed miR expression profiles in each PC sample therefore depend on the amount of tumour cells and stromal tissue. There is a risk that a pathological cancer miR expression was blurred by a small ratio of tumour cells compared to desmoplastic stroma and normal tissue in the sample. On the other hand our study better reflect daily clinical practice, where a fine needle biopsy is used to detect tumour cells in pancreas or metastasis. In the literature no miRs are yet described to be related to the development of fibrosis in pancreas.

The miR microarray used in the present study detects 664 different human miRs. Many of these miRs are discovered recently and are not analyzed in earlier miR studies of patients with pancreatic cancer. Few significant miRs described by others, e.g. miR-133a and miR-155, were not significantly differently expressed in tumour tissue and normal tissue in our study. Some significant miRs may not be detected in our analyses since we used non-micro-dissected tumour tissue.

In conclusion, we identified systematic differences in patterns of miR expression between pancreas tissues, including both cancer cells and stroma, obtained from patients with PC and A-AC compared to tissue from patients with chronic pancreatitis and normal pancreas. Some of the most differentially expressed miRs play a well described role in normal development, homeostasis or oncogenesis. We demonstrated that a panel of 19 miRs expressions could separate PC and A-AC tissues from non-neoplastic tissue samples with very high sensitivity, predictive value and accuracy. Fourteen of these miRs have not been related to diagnosis of PC before. We validated an earlier described diagnostic miR expression profile, i.e. the difference in miR-196 and miR-217. Furthermore, we identified three new combinations of five miRs (miR-411, miR-198, miR-614, miR-122 and miR-93*) which were even better to discriminate PC and A-AC tissue from chronic pancreatitis and normal pancreas. Prospective studies are needed to evaluate if this panel of miRs could have a role as biomarkers for early diagnosis of patients with pancreatic cancer.

### REFERENCE LIST

[1] Hidalgo M. Pancreatic cancer. N Engl J Med 2010; 362:1605-17.
[2] Li D, Xie K, Wolff R, Abbruzzese J L. Pancreatic cancer. Lancet 2004; 363:1049-57.
[3] Jemal A, Siegel R, Xu J, Ward E. Cancer Statistics, 2010. CA Cancer J Clin 2010.
[4] Wagner M, Redaelli C, Lietz M, Seiler C A, Friess H, Buchler M W. Curative resection is the single most important factor determining outcome in patients with pancreatic adenocarcinoma. Br J Surg 2004; 91:586-94.
[5] Riall T S, Cameron J L, Lillemoe K D, et al. Resected periampullary adenocarcinoma: 5-year survivors and their 6- to 10-year follow-up. Surgery 2006; 140:764-72.
[6] Beger H G, Thorab F C, Liu Z, Harada N, Rau B M. Pathogenesis and treatment of neoplastic diseases of the papilla of Vater: Kausch-Whipple procedure with lymph node dissection in cancer of the papilla of Vater. J Hepatobiliary Pancreat Surg 2004; 11:232-8.
[7] Bettschart V, Rahman M Q, Engelken F J, Madhavan K K, Parks R W, Garden O J. Presentation, treatment and outcome in patients with ampullary tumours. Br Surg 2004; 91:1600-7.
[8] Esquela-Kerscher A, Slack F J. Oncomirs—microRNAs with a role in cancer. Nat Rev Cancer 2006; 6:259-69.
[9] Hwang H W, Mendell J T. MicroRNAs in cell proliferation, cell death, and tumourigenesis. Br J Cancer 2006; 94:776-80.
[10] Waldman S A, Terzic A. Translating MicroRNA discovery into clinical biomarkers in cancer. JAMA 2007; 297:1923-5.
[11] Li M, Marin-Muller C, Bharadwaj U, Chow K H, Yao Q, Chen C. MicroRNAs: control and loss of control in human physiology and disease. World J Surg 2009; 33:667-84.
[12] Nelson K M, Weiss G J. MicroRNAs and cancer: past, present, and potential future. Mol Cancer Ther 2008; 7:3655-60.
[13] Rachagani S, Kumar S, Batra S K. MicroRNA in pancreatic cancer: pathological, diagnostic and therapeutic implications. Cancer Lett 2010; 292:8-16.
[14] Lu J, Getz G, Miska E A, et al. MicroRNA expression profiles classify human cancers. Nature 2005; 435:834-8.
[15] Volinia S, Calin G A, Liu C G, et al. A microRNA expression signature of human solid tumours defines cancer gene targets. Proc Natl Acad Sci USA 2006; 103:2257-61.
[16] Landgraf P, Rusu M, Sheridan R, et al. A mammalian microRNA expression atlas based on small RNA library sequencing. Cell 2007; 129:1401-14.
[17] Rosenfeld N, Aharonov R, Meiri E, et al. MicroRNAs accurately identify cancer tissue origin. Nat Biotechnol 2008; 26:462-9.
[18] Bloomston M, Frankel W L, Petrocca F, et al. MicroRNA expression patterns to differentiate pancreatic adenocarcinoma from normal pancreas and chronic pancreatitis. JAMA 2007; 297:1901-8.
[19] Szafranska A E, Davison T S, John J, et al. MicroRNA expression alterations are linked to tumourigenesis and non-neoplastic processes in pancreatic ductal adenocarcinoma. Oncogene 2007; 26:4442-52.
[20] Dillhoff M, Liu J, Frankel W, Croce C, Bloomston M. MicroRNA-21 is overexpressed in pancreatic cancer and a potential predictor of survival. J Gastrointest Surg 2008; 12:2171-6.
[21] Zhang Y, Li M, Wang H, et al. Profiling of 95 microRNAs in pancreatic cancer cell lines and surgical specimens by real-time PCR analysis. World J Surg 2009; 33:698-709.
[22] Habbe N, Koorstra J B, Mendell J T, et al. MicroRNA miR-155 is a biomarker of early pancreatic neoplasia. Cancer Biol Ther 2009; 8:340-6.
[23] lorio MV, Croce C M. MicroRNAs in cancer: small molecules with a huge impact. Clin Oncol 2009; 27:5848-56.
[24] Szafranska A E, Doleshal M, Edmunds H S, et al. Analysis of microRNAs in pancreatic fine-needle aspirates can classify benign and malignant tissues. Clin Chem 2008; 54:1716-24.
[25] Chu G C, Kimmelman A C, Hezel A F, DePinho R A. Stromal biology of pancreatic cancer. J Cell Biochem 2007; 101:887-907.
[26] Mar J C, Kimura Y, Schroder K, et al. Data-driven normalization strategies for high-throughput quantitative RT-PCR. BMC Bioinformatics 2009; 10:110.
[27] Friedman J, Hastie T, Tibshirani R. Sparse inverse covariance estimation with the graphical lasso. Biostatistics 2008; 9:432-41.
[28] Zhao J J, Yang J, Lin J, et al. Identification of miRNAs associated with tumourigenesis of retinoblastoma by miRNA microarray analysis. Childs Nerv Syst 2009; 25:13-20.
[29] Lee H C, Kim J G, Chae Y S, et al. Prognostic impact of microRNA-related gene polymorphisms on survival of patients with colorectal cancer. J Cancer Res Clin Oncol 2010; 136:1073-8.
[30] Tsai W C, Hsu P W, Lai T C, et al. MicroRNA-122, a tumour suppressor microRNA that regulates intrahepatic metastasis of hepatocellular carcinoma. Hepatology 2009; 49:1571-82.
[31] Pineau P, Volinia S, McJunkin K, et al. miR-221 overexpression contributes to liver tumourigenesis. Proc Natl Acad Sci USA 2010; 107:264-9.
[32] Zhao W G, Yu S N, Lu Z H, Ma Y H, Gu Y M, Chen J. The miR-217 microRNA functions as a potential tumour suppressor in pancreatic ductal adenocarcinoma by targeting KRAS. Carcinogenesis 2010; 31:1726-33.
[33] Yu S, Lu Z, Liu C, et al. miRNA-96 suppresses KRAS and functions as a tumour suppressor gene in pancreatic cancer. Cancer Res 2010; 70:6015-25.
[34] Hanoun N, Delpu Y, Suriawinata A A, et al. The silencing of microRNA 148a production by DNA hypermethylation is an early event in pancreatic carcinogenesis. Clin Chem 2010; 56:1107-18.
[35] Poy M N, Eliasson L, Krutzfeldt J, et al. A pancreatic islet-specific microRNA regulates insulin secretion. Nature 2004; 432:226-30.
[36] Poy M N, Hausser J, Trajkovski M, et al. miR-375 maintains normal pancreatic alpha- and beta-cell mass. Proc Natl Acad Sci USA 2009; 106:5813-8.
[37] Ho A S, Huang X, Cao H, et al. Circulating miR-210 as a Novel Hypoxia Marker in Pancreatic Cancer. Transl Oncol 2010; 3:109-13.
[38] Crosby M E, Kulshreshtha R, Ivan M, Glazer P M. MicroRNA regulation of DNA repair gene expression in hypoxic stress. Cancer Res 2009; 69:1221-9.
[39] Crosby M E, Devlin C M, Glazer P M, Calin G A, Ivan M. Emerging roles of microRNAs in the molecular responses to hypoxia. Curr Pharm Des 2009; 15:3861-6.
[40] Peter M E. Let-7 and miR-200 microRNAs: guardians against pluripotency and cancer progression. Cell Cycle 2009; 8:843-52.
[41] Gregory P A, Bert A G, Paterson E L, et al. The miR-200 family and miR-205 regulate epithelial to mesenchymal transition by targeting ZEB1 and SIP1. Nat Cell Biol 2008; 10:593-601.
[42] Gregory P A, Bracken C P, Bert A G, Goodall G J. MicroRNAs as regulators of epithelial-mesenchymal transition. Cell Cycle 2008; 7:3112-8.
[43] Hino K, Fukao T, Watanabe M. Regulatory interaction of HNF1-alpha to microRNA-194 gene during intestinal epithelial cell differentiation. Nucleic Acids Symp Ser (Oxf) 2007; 415-6.
[44] Hwang R F, Moore T, Arumugam T, et al. Cancer-associated stromal fibroblasts promote pancreatic tumour progression. Cancer Res 2008; 68:918-26.

TABLE 1
MicroRNAs significantly differently expressed in pancreatic cancer (PC), chronic pancreatitis (CH) and normal
pancreas. P-values are Bonferroni corrected for multiple testing. P-values, dm and fold change for the miRs which
are also found significantly differently expressed by Bloomston et al and Szafranska et al. are listed.

| | Corrected | | | Bloomston et al | Szafranska et al (2008) | Szafranska et al (2007) |
|---|---|---|---|---|---|---|
| | p-value | Dm (ΔΔCt)* | Fold change* | fold change | ΔΔCt (p-value) | Δh ** (p) |
| Normal vs. PC | | | | | | |
| hsa-miR-198 | 5.803E−36 | 4.953 | 30.980 | | | |
| hsa-miR-34c-5p | 6.182E−35 | 3.114 | 8.655 | | | |
| hsa-miR-21 | 1.144 × 10−24 | 2.134 | 4.390 | 3.08 | | |
| hsa-miR-708 | 5.722 × 10−24 | 2.190 | 4.564 | | | |
| hsa-miR-614 | 1.625 × 10−21 | 5.595 | 48.345 | | | |
| hsa-miR-196b | 2.784 × 10−21 | 4.396 | 21.057 | | miR-196-a 6.59 | 2.67 |
| | | | | | (1.39 × 10−6) | (3.79E−04) |
| hsa-miR-939 | 1.753 × 10−20 | 3.005 | 8.030 | | | |
| hsa-miR-148a | 1.798 × 10−20 | −3.721 | 0.076 | 0.18 | −6.15 | −3.3 |
| | | | | | (9.79 × 10−10) | (1.91E−10) |
| hsa-miR-801 | 2.347 × 10−19 | 2.949 | 7.721 | | | |
| hsa-miR-886-5p | 2.413 × 10−19 | 2.208 | 4.620 | | | |
| hsa-miR-210 | 8.209 × 10−19 | 2.310 | 4.958 | 2.97 | 2.31 | 2.82 |
| | | | | | (6.08 × 10−4) | (2.38E−08) |
| hsa-miR-31 | 9.463 × 10−15 | 2.989 | 7.941 | | 2.48 | 2.79 |
| | | | | | (1.96 × 10−2) | (1.52E−01) |
| hsa-miR-130b | 8.260 × 10−14 | −2.627 | 0.162 | | −3.86 | −2.43 |
| | | | | | (7.85 × 10−8) | (4.01E−08) |
| hsa-miR-100* | 1.665 × 10−13 | 2.745 | 6.704 | 2.49 | | |
| hsa-miR-222* | 1.881 × 10−13 | 2.101 | 4.291 | 2.70 | | miR-222 2.06 |
| | | | | | | (3.43E−11) |
| hsa-miR-375 | 9.778 × 10−13 | −3.226 | 0.107 | 0.46 | −6.20 | −2.51 |
| | | | | | (2.81 × 10−7) | (1.41E−11) |
| hsa-miR-494 | 1.510 × 10−12 | 2.083 | 4.237 | | | |
| hsa-miR-148a* | 1.604 × 10−12 | −5.166 | 0.028 | 0.18 | | |
| hsa-miR-141* | 1.883 × 10−8 | −2.640 | 0.160 | | | miR-141 −1.5 |
| | | | | | | (1.06E−05) |
| hsa-miR-217 | 4.833 × 10−8 | −5.372 | 0.024 | | −10.81 | −5.68 |
| | | | | | (5.85 × 10−10) | (3.81E−13) |
| hsa-miR-154* | 7.379 × 10−8 | −3.492 | 0.089 | | | |
| hsa-miR-411* | 7.737 × 10−7 | −3.016 | 0.124 | | | |
| hsa-miR-216b | 2.484 × 10−6 | −5.473 | 0.023 | | | miR-216 −5.45 |
| | | | | | | (4.56E−13) |
| hsa-miR-379* | 2.650 × 10−6 | −2.937 | 0.131 | | | |
| hsa-miR-216a | 2.899 × 10−6 | −4.670 | 0.038 | | | miR-216 −5.45 |
| | | | | | | (4.56E−13) |
| hsa-miR-143* | 1.790 × 10−5 | 4.820 | 28.253 | miR-143 2.19 | miR-143 1.36 | miR-143 1.94 |
| | | | | | (9.12 × 10−3) | (<1E−11) |
| hsa-miR-19a* | 5.162 × 10−5 | −2.691 | 0.155 | | | |
| hsa-miR-377* | 1.326 × 10−4 | −2.720 | 0.152 | | | |
| hsa-miR-200c* | 1.290 × 10−3 | −2.190 | 0.219 | | | |
| hsa-miR-374a* | 1.928 × 10−3 | −2.279 | 0.206 | | | |
| hsa-miR-205 | 7.105 × 10−3 | 4.314 | 19.897 | 2.24 | 1.70 | 2.22/9 |
| | | | | | (4.29 × 10−1) | (3.47E−06) |
| hsa-miR-194* | 0.029 | −3.055 | 0.120 | | | |
| CH vs. PC | | | | | | |
| hsa-miR-614 | 2.703 × 10−16 | 5.058 | 33.305 | | | |
| hsa-miR-492 | 1.113 × 10−12 | 7.377 | 166.230 | | | |
| hsa-miR-622 | 1.245 × 10−12 | 2.777 | 6.855 | | | |
| hsa-miR-135b* | 7.767 × 10−11 | 3.461 | 11.015 | | | |
| hsa-miR-196b | 4.649 × 10−10 | 3.064 | 8.362 | | miR-196a 4.69 | 1.64 |
| | | | | | (8.00 × 10−5) | (2.22E−4) |
| hsa-miR-198 | 1.089 × 10−9 | 2.385 | 5.224 | | | |
| hsa-miR-516a-3p | 1.116 × 10−6 | 2.057 | 4.160 | | | |
| hsa-miR-122 | 2.679 × 10−6 | −3.790 | 0.072 | | | |
| hsa-miR-509-5p | 5.692 × 10−6 | 3.327 | 10.033 | | | |
| hsa-miR-147b | 3.730 × 10−5 | 3.312 | 9.931 | | | |
| hsa-miR-148a | 6.323 × 10−5 | −2.101 | 0.233 | 0.22 | −4.28 | −1.98 |
| | | | | | (2.01 × 10−10) | (2.83E−05) |
| hsa-miR-125b-2* | 3.031 × 10−4 | −2.244 | 0.211 | | | |
| hsa-miR-377* | 1.355 × 10−3 | −2.694 | 0.154 | | | |
| hsa-miR-154* | 3.682 × 10−3 | −2.692 | 0.155 | | | |
| hsa-miR-379* | 4.372 × 10−3 | −2.376 | 0.193 | | | |
| hsa-miR-411* | 5.362 × 10−3 | −2.357 | 0.195 | | | |
| hsa-miR-205 | 8.262 × 10−3 | 4.678 | 25.591 | | 3.34 | |
| | | | | | (1.30 × 10−1) | |
| hsa-miR-374a* | 0.046 | −2.105 | 0.232 | | | |
| Normal vs. CH | | | | | | |
| hsa-miR-194* | 1.579 × 10−5 | −4.270 | 0.052 | miR194 1.69 | | |
| hsa-miR-141* | 6.164 × 10−4 | −2.261 | 0.209 | | | |
| hsa-miR-198 | 1.848 × 10−3 | 2.568 | 5.930 | 1.78 | | |
| hsa-miR-130b* | 2.965 × 10−3 | −2.076 | 0.237 | | miR-130b 1.41 | |
| | | | | | (7.13 × 10−3) | |
| hsa-miR-650 | 5.100 × 10−3 | 2.736 | 6.662 | | | |
| hsa-miR-219-1-3p | 0.021 | −2.621 | 0.163 | | | |
| hsa-miR-766 | 0.022 | 2.564 | 5.914 | | | |

*The difference of means (dm) column corresponds to the log2 fold change and is the difference between class means (1st tissue − 2nd tissue mean Ct values). If the value is positive, it means that the average Ct is higher in the 1st tissue class and thus, the miR is expressed at lower levels in the 1st class. The fold change is 2^{ΔΔCt} for qPCR data, i.e. if dm = −1, then the miR is 2 times up-regulated in the 2nd tissue compared to the 1st tissue while dm = −1 means that the miR is 2 times down-regulated in the 2nd tissue compared to the 1st tissue.
** Δh corresponds to the dm-value used in our study and the other study by Szafranska et al.

TABLE 2
Lasso classifier features and their stability in 1st 10-fold cross
validation dataset.

| Feature | Stability (1-10) |
|---|---|
| hsa-miR-122 | 6 |
| hsa-miR-135b | 10 |
| hsa-miR-135b* | 10 |
| hsa-miR-136* | 9 |
| hsa-miR-186 | 10 |
| hsa-miR-196b | 10 |
| hsa-miR-198 | 10 |
| hsa-miR-203 | 5 |
| hsa-miR-222 | 10 |
| hsa-miR-23a | 5 |
| hsa-miR-34c-5p | 8 |
| hsa-miR-451 | 10 |
| hsa-miR-490-3p | 5 |
| hsa-miR-492 | 10 |
| hsa-miR-509-5p | 9 |
| hsa-miR-571 | 10 |
| hsa-miR-614 | 10 |
| hsa-miR-622 | 10 |
| hsa-miR-939 | 8 |

TABLE 3
Differences in expressions of two microRNAs as biomarkers to
differentiate between pancreatic cancer and controls

| MicroRNA | | | |
|---|---|---|---|
| (difference) | PC vs. CH | PC vs. NP | PC + A-AC vs. CH + NP |
| mirR196b + miR217 | 3.52e−7 | 8.59e−20 | 4.35e−24 |
| mirR411 + miR198 | 2.63e−13 | 5.17e−43 | 4.64e−49 |
| mirR614 + miR122 | 7.76e−18 | 1.62e−14 | 8.64e−38 |
| mirR614 + miR93* | 9.01e−18 | 5.56e−24 | 2.64e−42 |

PC, pancreatic cancer;
A-AC, ampullary adenocarcinoma;
CH, chronic pancreatitis;
NP, normal pancreas.

TABLE 4
All microRNAs significantly differently expressed in PC, ampullary adenocarcinomas (A-AC),
chronic pancreatitis (CH) and normal pancreas. P-values in this table are not corrected
for multiple testing, but only miRs significantly expressed (p < 0.05) after Bonferroni
correction are included. P-values, dm and fold change for the miRs which are also found
significantly different expressed by Bloomston et al and Szafranska et al are listed.

| | | | | Bloomston et al | Szafranska et al | Szafranska et al |
|---|---|---|---|---|---|---|
| | p-value | dm | Fold change | fold change | ΔΔCt (p-value) | Δh (p) |
| Normal vs. PC | | | | | | |
| hsa-miR-198 | 1.222E−38 | 4.953 | 30.980 | | | |
| hsa-miR-34c-5p | 1.301E−37 | 3.114 | 8.655 | | | |
| hsa-miR-21 | 2.409E−27 | 2.134 | 4.390 | 3.08 | | |
| hsa-miR-708 | 1.205E−26 | 2.190 | 4.564 | | | |
| hsa-miR-614 | 3.421E−24 | 5.595 | 48.345 | | | |
| hsa-miR-196b | 5.861E−24 | 4.396 | 21.057 | | miR-196-a 6.59 | 2.67 |
| | | | | | (1.39 × 10−6) | (3.79E−04) |
| hsa-miR-939 | 3.690E−23 | 3.005 | 8.030 | | | |
| hsa-miR-148a | 3.785E−23 | −3.721 | 0.076 | 0.18 | −6.15 | −3.3 |
| | | | | | (9.79 × 10−10) | (1.91E−10) |
| hsa-miR-801 | 4.942E−22 | 2.949 | 7.721 | | | |
| hsa-miR-886-5p | 5.080E−22 | 2.208 | 4.620 | | | |
| hsa-miR-210 | 1.728E−21 | 2.310 | 4.958 | 2.97 | 2.31 | 2.82 |
| | | | | | (6.08 × 10−4) | (2.38E−08) |
| hsa-miR-190b | 3.789E−21 | −3.001 | 0.125 | | | |
| hsa-miR-142-3p | 9.153E−21 | 2.166 | 4.488 | | | |
| hsa-miR-130b* | 2.347E−20 | −3.987 | 0.063 | | | |
| hsa-miR-649 | 3.987E−20 | 3.727 | 13.246 | | | |
| hsa-miR-30a* | 3.459E−19 | −2.003 | 0.250 | | | |
| hsa-miR-650 | 1.272E−18 | 3.467 | 11.055 | | | |
| hsa-miR-492 | 6.125E−18 | 7.384 | 166.990 | | | |
| hsa-miR-922 | 7.592E−18 | 3.994 | 15.937 | | | |
| hsa-miR-31 | 1.992E−17 | 2.989 | 7.941 | | 2.48 | 2.79 |
| | | | | | (1.96 × 10−2) | (1.52E−01) |
| hsa-miR-219-1-3p | 1.036E−16 | −4.150 | 0.056 | | | |
| hsa-miR-432* | 1.201E−16 | −4.294 | 0.051 | | | |
| hsa-miR-130b | 1.739E−16 | −2.627 | 0.162 | | −3.86 | −2.43 |
| | | | | | (7.85 × 10−8) | (4.01E−08) |
| hsa-miR-100* | 3.505E−16 | 2.745 | 6.704 | 2.49 | | |
| hsa-miR-222* | 3.959E−16 | 2.101 | 4.291 | 2.70 | | miR-222 2.06 |
| | | | | | | (3.43E−11) |
| hsa-miR-375 | 2.059E−15 | −3.226 | 0.107 | 0.46 | −6.20 | −2.51 |
| | | | | | (2.81 × 10−7) | (1.41E−11) |
| hsa-miR-135b* | 2.364E−15 | 3.454 | 10.959 | | | |
| hsa-miR-592 | 2.783E−15 | −2.140 | 0.227 | | | |
| hsa-miR-494 | 3.180E−15 | 2.083 | 4.237 | | | |
| hsa-miR-148a* | 3.377E−15 | −5.166 | 0.028 | 0.18 | | |
| hsa-miR-635 | 5.813E−15 | 2.566 | 5.920 | | | |
| hsa-miR-598 | 5.931E−15 | −2.142 | 0.227 | | | |
| hsa-miR-622 | 2.206E−14 | 2.491 | 5.623 | | | |
| hsa-miR-877 | 4.509E−13 | 2.008 | 4.021 | | | |
| hsa-miR-875-5p | 1.180E−12 | 2.036 | 4.102 | | | |
| hsa-miR-451 | 1.863E−12 | 2.534 | 5.793 | | | |
| hsa-miR-891a | 2.297E−12 | −4.875 | 0.034 | | | |
| hsa-miR-509-5p | 3.820E−12 | 3.908 | 15.007 | | | |
| RNU48 | 1.086E−11 | −2.190 | 0.219 | | | |
| hsa-miR-518d-3p | 1.096E−11 | −3.938 | 0.065 | | | |
| hsa-miR-648 | 2.151E−11 | 3.110 | 8.635 | | | |
| hsa-miR-449b | 2.229E−11 | −2.725 | 0.151 | | | |
| hsa-miR-141* | 3.964E−11 | −2.640 | 0.160 | | | miR-141 −1.5 |
| | | | | | | (1.06E−05) |
| hsa-miR-643 | 4.665E−11 | 2.593 | 6.034 | | | |
| hsa-miR-575 | 6.107E−11 | 2.910 | 7.514 | | | |
| hsa-miR-193b* | 6.689E−11 | −3.044 | 0.121 | | | |
| hsa-miR-217 | 1.017E−10 | −5.372 | 0.024 | | −10.81 | −5.68 |
| | | | | | (5.85 × 10−10) | (3.81E−13) |
| hsa-miR-154* | 1.553E−10 | −3.492 | 0.089 | | | |
| hsa-miR-34b* | 1.927E−10 | 2.662 | 6.327 | | | |
| hsa-miR-7-2* | 3.445E−10 | −3.876 | 0.068 | | | |
| hsa-miR-147b | 9.704E−10 | 3.374 | 10.365 | | | |
| hsa-miR-584 | 1.092E−09 | 3.378 | 10.393 | | | |
| hsa-miR-449a | 1.253E−09 | −2.605 | 0.164 | | | |
| hsa-miR-411* | 1.629E−09 | −3.016 | 0.124 | | | |
| hsa-miR-589* | 1.692E−09 | −3.555 | 0.085 | | | |
| RNU6B | 3.546E−09 | −3.383 | 0.096 | | | |
| hsa-miR-216b | 5.230E−09 | −5.473 | 0.023 | | | miR-216 −5.45 |
| | | | | | | (4.56E−13) |
| hsa-miR-379* | 5.579E−09 | −2.937 | 0.131 | | | |
| hsa-miR-216a | 6.104E−09 | −4.670 | 0.038 | | | miR-216 −5.45 |
| | | | | | | (4.56E−13) |
| hsa-miR-219-5p | 7.613E−09 | −2.918 | 0.132 | | | |
| hsa-miR-486-3p | 1.286E−08 | −3.050 | 0.121 | | | |
| hsa-miR-153 | 1.457E−08 | −2.831 | 0.141 | | | |
| hsa-miR-143* | 3.768E−08 | 4.820 | 28.253 | miR-143 2.19 | miR-143 1.36 | miR-143 1.94 |
| | | | | | (9.12 × 10−3) | (<1E−11) |
| hsa-miR-542-5p | 5.920E−08 | 2.452 | 5.470 | | | |
| hsa-miR-644 | 6.572E−08 | 2.717 | 6.576 | | | |
| hsa-miR-944 | 7.423E−08 | 3.145 | 8.848 | | | |
| hsa-miR-129-5p | 7.576E−08 | −3.289 | 0.102 | | | |
| hsa-miR-19a* | 1.087E−07 | −2.691 | 0.155 | | | |
| hsa-miR-377* | 2.792E−07 | −2.720 | 0.152 | | | |
| hsa-miR-640 | 3.933E−07 | 2.853 | 7.224 | | | |
| hsa-miR-383 | 5.406E−07 | −2.662 | 0.158 | | | |
| hsa-miR-208 | 1.145E−06 | 2.935 | 7.650 | | | |
| hsa-miR-566 | 2.228E−06 | 2.965 | 7.809 | | | |
| hsa-miR-200c* | 2.716E−06 | −2.190 | 0.219 | | | |
| hsa-miR-147 | 2.961E−06 | 2.716 | 6.568 | | | |
| hsa-miR-374a* | 4.059E−06 | −2.279 | 0.206 | | | |
| hsa-miR-92b* | 5.483E−06 | 2.472 | 5.548 | | | |
| hsa-miR-888 | 6.067E−06 | −2.174 | 0.222 | | | |
| hsa-miR-205 | 1.496E−05 | 4.314 | 19.897 | 2.24 | 1.70 | 2.22 |
| | | | | | (4.29 × 10−1) | (3.47E−06) |
| hsa-miR-129-3p | 4.681E−05 | −3.066 | 0.119 | | | |
| hsa-miR-499-5p | 5.060E−05 | −2.042 | 0.243 | | | |
| hsa-miR-194* | 6.207E−05 | −3.055 | 0.120 | | | |
| hsa-miR-543 | 8.397E−05 | −2.198 | 0.218 | | | |
| hsa-miR-554 | 9.081E−05 | 2.182 | 4.537 | | | |
| CH vs. PC | | | | | | |
| hsa-miR-614 | 5.690E−19 | 5.058 | 33.305 | | | |
| hsa-miR-492 | 2.343E−15 | 7.377 | 166.230 | | | |
| hsa-miR-622 | 2.622E−15 | 2.777 | 6.855 | | | |
| hsa-miR-135b* | 1.635E−13 | 3.461 | 11.015 | | | |
| hsa-miR-196b | 9.787E−13 | 3.064 | 8.362 | | miR-196a 4.69 | 1.64 |
| | | | | | (8.00 × 10−5) | (2.22E−4) |
| hsa-miR-198 | 2.292E−12 | 2.385 | 5.224 | | | |
| hsa-miR-516a-3p | 2.349E−09 | 2.057 | 4.160 | | | |
| hsa-miR-122 | 5.639E−09 | −3.790 | 0.072 | | | |
| hsa-miR-509-5p | 1.198E−08 | 3.327 | 10.033 | | | |
| hsa-miR-147b | 7.185E−08 | 3.312 | 9.931 | | | |
| hsa-miR-148a | 1.331E−07 | −2.101 | 0.233 | 0.22 | −4.28 | −1.98 |
| | | | | | (2.01 × 10−10) | (2.83E−5) |
| hsa-miR-648 | 1.848E−07 | 2.572 | 5.947 | | | |
| hsa-miR-643 | 3.758E−07 | 2.034 | 4.097 | | | |
| hsa-miR-125b-2* | 6.382E−07 | −2.244 | 0.211 | | | |
| hsa-miR-432* | 7.176E−07 | −2.695 | 0.154 | | | |
| hsa-miR-575 | 1.937E−06 | 2.269 | 4.821 | | | |
| hsa-miR-520c-3p | 2.581E−06 | 2.214 | 4.639 | | | |
| hsa-miR-584 | 2.746E−06 | 2.769 | 6.816 | | | |
| hsa-miR-377* | 2.853E−06 | −2.694 | 0.154 | | | |
| hsa-miR-148a* | 3.135E−06 | −3.242 | 0.106 | | | |
| hsa-miR-891a | 3.482E−06 | −3.516 | 0.087 | | | |
| hsa-miR-337-3p | 4.109E−06 | −2.990 | 0.126 | | | |
| hsa-miR-154* | 7.751E−06 | −2.692 | 0.155 | | | |
| hsa-miR-379* | 9.203E−06 | −2.376 | 0.193 | | | |
| hsa-miR-411* | 1.129E−05 | −2.357 | 0.195 | | | |
| hsa-miR-205 | 1.739E−05 | 4.678 | 25.591 | | 3.34 | |
| | | | | | (1.30 × 10−1) | |
| hsa-miR-208 | 1.995E−05 | 2.851 | 7.215 | | | |
| RNU6B | 2.828E−05 | −2.571 | 0.168 | | | |
| hsa-miR-493* | 4.226E−05 | −2.568 | 0.169 | | | |
| hsa-miR-7-2* | 4.232E−05 | −2.693 | 0.155 | | | |
| hsa-miR-512-3p | 4.673E−05 | 2.136 | 4.394 | | | |
| hsa-miR-193b* | 5.576E−05 | −2.043 | 0.243 | | | |
| hsa-miR-374a* | 9.703E−05 | −2.105 | 0.232 | | | |
| A-AC vs. PC | | | | | | |
| hsa-miR-194* | 1.849E−24 | −5.467 | 0.023 | | | |
| hsa-miR-187 | 1.542E−13 | −4.726 | 0.038 | | | |
| hsa-miR-654-5p | 2.233E−09 | 2.094 | 4.270 | | | |
| hsa-miR-552 | 5.041E−09 | −3.112 | 0.116 | | | |
| hsa-miR-205 | 4.694E−05 | 2.624 | 6.163 | | | |
| Normal vs. A-AC | | | | | | |
| hsa-miR-198 | 1.071E−27 | 4.278 | 19.400 | | | |
| hsa-miR-10a | 2.530E−27 | 2.106 | 4.306 | | | |
| hsa-miR-650 | 3.280E−27 | 4.340 | 20.245 | | | |
| hsa-miR-34c-5p | 5.508E−26 | 2.692 | 6.462 | | | |
| hsa-miR-30a* | 1.434E−25 | −2.418 | 0.187 | | | |
| hsa-miR-492 | 8.001E−25 | 8.582 | 383.318 | | | |
| hsa-miR-148a | 8.210E−24 | −3.598 | 0.0826 | | | |
| hsa-miR-30e* | 1.480E−23 | −2.244 | 0.211 | | | |
| hsa-miR-801 | 2.752E−23 | 3.322 | 10.000 | | | |
| hsa-miR-614 | 8.103E−23 | 6.239 | 75.555 | | | |
| hsa-miR-649 | 4.450E−22 | 4.308 | 19.803 | | | |
| hsa-miR-143 | 9.547E−22 | 2.064 | 4.183 | | | |
| hsa-miR-323-3p | 2.533E−21 | −2.022 | 0.246 | | | |
| hsa-miR-939 | 3.711E−21 | 3.068 | 8.386 | | | |
| hsa-miR-130b* | 1.044E−20 | −3.519 | 0.087 | | | |
| hsa-miR-335 | 1.115E−20 | −2.264 | 0.208 | | | |
| hsa-miR-30c | 1.195E−20 | −2.069 | 0.238 | | | |
| hsa-miR-31 | 1.590E−20 | 3.194 | 9.151 | | | |
| hsa-miR-147b | 2.942E−20 | 4.436 | 21.649 | | | |
| hsa-miR-130b | 4.314E−20 | −2.820 | 0.142 | | | |
| hsa-miR-210 | 7.733E−20 | 2.163 | 4.477 | | | |
| hsa-miR-922 | 1.090E−19 | 4.376 | 20.760 | | | |
| hsa-miR-622 | 2.175E−19 | 3.230 | 9.382 | | | |
| hsa-miR-548b-5p | 2.684E−19 | 2.165 | 4.484 | | | |
| hsa-miR-142-3p | 2.896E−19 | 2.205 | 4.610 | | | |
| hsa-miR-891a | 2.009E−18 | −6.181 | 0.014 | | | |
| hsa-miR-196b | 6.028E−18 | 5.089 | 34.041 | | | |
| hsa-miR-135b* | 9.602E−18 | 4.064 | 16.730 | | | |
| hsa-miR-133b | 1.266E−17 | 2.641 | 6.238 | | | |
| hsa-miR-590-5p | 1.715E−17 | −2.138 | 0.227 | | | |
| hsa-miR-494 | 4.378E−17 | 2.366 | 5.156 | | | |
| hsa-miR-432* | 1.070E−16 | −4.516 | 0.044 | | | |
| hsa-miR-133a | 1.193E−16 | 2.463 | 5.512 | | | |
| hsa-miR-190b | 4.543E−16 | −2.881 | 0.136 | | | |
| hsa-miR-135b | 1.131E−15 | 2.139 | 4.490 | | | |
| hsa-miR-548d-5p | 1.330E−15 | 2.020 | 4.055 | | | |
| hsa-miR-598 | 1.460E−15 | −2.476 | 0.180 | | | |
| hsa-miR-923 | 3.897E−15 | 2.246 | 4.743 | | | |
| hsa-miR-143* | 9.515E−15 | 6.886 | 118.237 | | | |
| hsa-miR-604 | 1.098E−14 | 2.184 | 4.543 | | | |
| hsa-miR-148a* | 2.172E−14 | −4.359 | 0.048 | | | |
| hsa-miR-411* | 3.799E−14 | −4.100 | 0.058 | | | |
| hsa-miR-7-2* | 2.883E−13 | −5.065 | 0.023 | | | |
| hsa-miR-551b* | 5.218E−13 | 2.050 | 4.140 | | | |
| hsa-miR-644 | 6.766E−13 | 3.607 | 12.189 | | | |
| hsa-miR-379* | 1.225E−12 | −3.945 | 0.065 | | | |
| hsa-miR-639 | 3.556E−12 | 2.884 | 7.383 | | | |
| hsa-miR-643 | 4.604E−12 | 2.460 | 5.504 | | | |
| hsa-miR-487b | 5.427E−12 | −2.214 | 0.216 | | | |
| hsa-miR-575 | 5.462E−12 | 3.277 | 9.690 | | | |
| hsa-miR-375 | 6.319E−12 | −2.763 | 0.147 | | | |
| hsa-miR-635 | 7.068E−12 | 2.835 | 7.056 | | | |
| hsa-miR-187 | 8.352E−12 | 6.229 | 75.020 | | | |
| hsa-miR-875-5p | 2.504E−11 | 2.249 | 4.754 | | | |
| hsa-miR-154* | 1.064E−10 | −3.743 | 0.075 | | | |
| hsa-miR-888 | 1.60E−10 | −3.338 | 0.099 | | | |
| hsa-miR-937 | 2.445E−10 | 3.677 | 12.788 | | | |
| hsa-miR-203 | 3.011E−10 | 2.010 | 4.029 | | | |
| hsa-miR-449b | 5.703E−10 | −2.792 | 0.144 | | | |
| hsa-miR-640 | 6.305E−10 | 3.220 | 9.317 | | | |
| hsa-miR-147 | 9.659E−10 | 3.652 | 12.570 | | | |
| hsa-miR-518d-3p | 1.098E−09 | −3.712 | 0.076 | | | |
| hsa-miR-648 | 1.135E−09 | 2.947 | 7.713 | | | |
| hsa-miR-33a* | 1.502E−09 | −2.406 | 0.189 | | | |
| hsa-miR-656 | 1.778E−09 | −2.305 | 0.202 | | | |
| hsa-miR-129-3p | 2.077E−09 | −4.663 | 0.039 | | | |
| hsa-miR-217 | 2.684E−09 | −5.613 | 0.020 | | | |
| hsa-miR-153 | 2.810E−09 | −3.326 | 0.100 | | | |
| hsa-miR-654-5p | 6.200E−09 | −3.364 | 0.097 | | | |
| RNU6B | 1.180E−08 | −3.014 | 0.124 | | | |
| hsa-miR-193b* | 1.302E−08 | −3.659 | 0.079 | | | |
| hsa-miR-451 | 1.358E−08 | 2.231 | 4.695 | | | |
| hsa-miR-219-1-3p | 1.745E−08 | −2.993 | 0.126 | | | |
| hsa-miR-616* | 1.819E−08 | −2.233 | 0.213 | | | |
| hsa-miR-490-3p | 1.856E−08 | 2.092 | 4.264 | | | |
| hsa-miR-584 | 2.409E−08 | 3.201 | 9.195 | | | |
| hsa-miR-889 | 2.965E−08 | −2.205 | 0.217 | | | |
| hsa-miR-589* | 5.354E−08 | −3.283 | 0.103 | | | |
| hsa-miR-628-3p | 5.615E−08 | −2.455 | 0.182 | | | |
| hsa-miR-509-5p | 5.685E−08 | 3.065 | 8.368 | | | |
| hsa-miR-216a | 7.935E−08 | −5.256 | 0.026 | | | |
| hsa-miR-216b | 1.121E−07 | −6.207 | 0.014 | | | |
| hsa-miR-449a | 1.228E−07 | −2.646 | 0.160 | | | |
| hsa-miR-208 | 1.677E−07 | 3.206 | 9.226 | | | |
| hsa-miR-129-5p | 2.708E−07 | −3.465 | 0.091 | | | |
| hsa-miR-377* | 3.027E−07 | −2.725 | 0.151 | | | |
| hsa-miR-486-3p | 4.840E−07 | −3.005 | 0.123 | | | |
| hsa-miR-455-3p | 1.169E−06 | −2.320 | 0.200 | | | |
| hsa-miR-184 | 1.228E−06 | −2.415 | 0.187 | | | |
| hsa-miR-672 | 2.574E−06 | 2.770 | 6.821 | | | |
| hsa-miR-19a* | 3.149E−06 | −2.464 | 0.181 | | | |
| hsa-miR-219-5p | 3.540E−06 | −2.406 | 0.189 | | | |
| hsa-miR-154 | 5.347E−06 | −2.315 | 0.201 | | | |
| hsa-miR-518e | 5.547E−06 | 2.192 | 4.569 | | | |
| hsa-miR-374a* | 5.816E−06 | −2.487 | 0.178 | | | |
| hsa-miR-373 | 1.261E−05 | 2.739 | 6.676 | | | |
| hsa-miR-582-3p | 1.582E−05 | 2.340 | 5.063 | | | |
| hsa-miR-124 | 1.617E−05 | 2.033 | 4.091 | | | |
| hsa-let-7a* | 2.110E−05 | −2.005 | 0.249 | | | |
| hsa-miR-551b | 2.272E−05 | −2.133 | 0.228 | | | |
| hsa-miR-122 | 2.956E−05 | −2.334 | 0.198 | | | |
| hsa-miR-543 | 3.383E−05 | −2.498 | 0.177 | | | |
| hsa-miR-337-3p | 3.874E−05 | −2.433 | 0.185 | | | |
| hsa-miR-493* | 3.927E−05 | −2.520 | 0.174 | | | |
| hsa-miR-944 | 4.116E−05 | 2.408 | 5.308 | | | |
| hsa-miR-552 | 4.837E−05 | 4.260 | 19.154 | | | |
| hsa-miR-497* | 5.182E−05 | −2.278 | 0.206 | | | |
| hsa-miR-513-3p | 6.205E−05 | 2.330 | 5.028 | | | |
| hsa-miR-554 | 9.333E−05 | 2.400 | 5.275 | | | |
| hsa-miR-330-5p | 9.472E−05 | 2.180 | 4.530 | | | |
| CH vs. A-AC | | | | | | |
| hsa-miR-492 | 1.396E−21 | 8.576 | 383.421 | | | |
| hsa-miR-622 | 2.612E−20 | 3.516 | 11.437 | | | |
| hsa-miR-614 | 6.228E−19 | 5.702 | 52.049 | | | |
| hsa-miR-147b | 6.070E−16 | 4.375 | 20.743 | | | |
| hsa-miR-135b* | 6.091E−16 | 4.072 | 16.815 | | | |
| hsa-miR-215 | 6.331E−15 | 3.037 | 8.207 | | | |
| hsa-miR-194* | 1.470E−13 | 6.682 | 102.711 | | | |
| hsa-miR-135b | 8.533E−13 | 2.026 | 4.072 | | | |
| hsa-miR-203 | 4.425E−12 | 2.660 | 6.321 | | | |
| hsa-miR-194 | 5.837E−12 | 2.338 | 5.055 | | | |
| hsa-miR-192 | 9.373E−12 | 2.241 | 4.726 | | | |
| hsa-miR-516a-3p | 1.293E−11 | 2.050 | 4.141 | | | |
| hsa-miR-133a | 3.839E−11 | 2.064 | 4.181 | | | |
| hsa-miR-196b | 1.213E−10 | 3.757 | 13.518 | | | |
| hsa-miR-891a | 2.033E−10 | −4.822 | 0.035 | | | |
| hsa-miR-133b | 4.165E−10 | −2.069 | 4.195 | | | |
| hsa-miR-649 | 8.478E−10 | 2.410 | 5.313 | | | |
| hsa-miR-654-5p | 9.579E−10 | −3.947 | 0.065 | | | |
| hsa-miR-122 | 9.937E−10 | −3.955 | 0.064 | | | |
| hsa-miR-411* | 1.463E−09 | −3.442 | 0.092 | | | |
| hsa-miR-125b-2* | 2.103E−09 | −3.227 | 0.107 | | | |
| hsa-miR-490-3p | 3.391E−09 | 2.673 | 6.378 | | | |
| hsa-miR-379* | 4.442E−09 | −3.384 | 0.096 | | | |
| hsa-miR-187 | 5.383E−08 | 5.393 | 42.025 | | | |
| hsa-miR-450b-5p | 5.835E−08 | −3.243 | 0.106 | | | |
| hsa-miR-7-2* | 7.857E−08 | −3.882 | 0.068 | | | |
| hsa-miR-656 | 1.063E−07 | −2.252 | 0.210 | | | |
| hsa-miR-337-3p | 1.396E−07 | −3.524 | 0.087 | | | |
| hsa-miR-575 | 1.506E−07 | 2.636 | 6.217 | | | |
| hsa-miR-432* | 1.806E−07 | −2.917 | 0.132 | | | |
| hsa-miR-493* | 2.734E−07 | −3.468 | 0.090 | | | |
| hsa-miR-937 | 3.739E−07 | 3.213 | 9.273 | | | |
| hsa-miR-888 | 7.074E−07 | −2.742 | 0.150 | | | |
| hsa-miR-376b | 1.137E−06 | −3.028 | 0.123 | | | |
| hsa-miR-520c-3p | 1.138E−06 | 2.345 | 5.080 | | | |
| hsa-miR-497* | 1.304E−06 | −2.902 | 0.134 | | | |
| hsa-miR-518e | 1.643E−06 | 2.596 | 6.048 | | | |
| hsa-miR-129-3p | 1.679E−06 | −4.020 | 0.062 | | | |
| hsa-miR-512-3p | 1.764E−06 | 2.602 | 6.071 | | | |
| hsa-miR-648 | 1.974E−06 | 2.409 | 5.312 | | | |
| hsa-miR-639 | 2.346E−06 | 2.142 | 4.393 | | | |
| hsa-miR-377* | 2.522E−06 | −2.670 | 0.154 | | | |
| hsa-miR-154* | 4.373E−06 | −2.943 | 0.130 | | | |
| hsa-miR-208 | 4.534E−06 | 3.121 | 8.700 | | | |
| hsa-miR-143* | 4.735E−06 | 4.354 | 20.456 | | | |
| hsa-miR-635 | 5.627E−06 | 2.025 | 4.069 | | | |
| hsa-miR-644 | 6.208E−06 | 2.343 | 5.072 | | | |
| hsa-miR-147 | 9.783E−06 | 2.921 | 7.574 | | | |
| hsa-miR-509-5p | 1.257E−05 | 2.484 | 5.594 | | | |
| hsa-miR-518f | 1.312E−05 | 2.281 | 4.860 | | | |
| hsa-miR-922 | 1.542E−05 | 2.234 | 4.704 | | | |
| hsa-miR-584 | 1.695E−05 | 2.592 | 6.030 | | | |
| hsa-miR-148a* | 4.588E−05 | −2.435 | 0.185 | | | |
| hsa-miR-552 | 6.004E−05 | 4.619 | 24.579 | | | |
| RNU6B | 7.572E−05 | −2.202 | 0.217 | | | |
| hsa-miR-154 | 7.767E−05 | −2.191 | 0.219 | | | |
| hsa-miR-543 | 1.014 × 10−4 | −2.458 | 0.182 | | | |
| Normal vs. CH | | | | | | |
| hsa-miR-194* | 2.106E−07 | −4.270 | 0.052 | miR194 1.69 | | |
| hsa-miR-141* | 1.298E−06 | −2.261 | 0.209 | | | miR-141 |
| hsa-miR-198 | 3.891E−06 | 2.568 | 5.930 | 1.78 | | |
| hsa-miR-130b* | 6.243E−06 | −2.076 | 0.237 | | miR-130b 1.41 | miR-130b |
| | | | | | (7.13 × 10−3) | |
| hsa-miR-650 | 1.074E−05 | 2.736 | 6.662 | | | |
| hsa-miR-219-1-3p | 4.458E−05 | −2.621 | 0.163 | | | |
| hsa-miR-766 | 4.639E−05 | 2.564 | 5.914 | | | |

1.-78. (canceled)

79. A method for diagnosing if an individual has, or is at risk of developing, pancreatic carcinoma, comprising determining the expression level of at least one miRNA in a sample obtained from said individual, wherein the expression level of said at least one miRNA comprises:
a. miR-411 and miR-198; or
b. miR-614 and miR-122; or
c. miR-614 and miR-93*; or
d. miR-614; or
e. miR-122; or
f. miR-93*; or
g. miR-198;
wherein the miRNA expression level of said one or more miRNAs, and/or the difference in the miRNA expression level of at least two miRNAs, is indicative of said individual having, or being at risk of developing, pancreatic carcinoma.

80. The method according to claim 79, further comprising determining the expression level of at least one miRNA selected from the group consisting of:
a. miR-198, miR-34c-5p, miR-614, miR-492, miR-10a, miR-622, miR-196b, miR-210, miR-939, miR-649, miR-801, miR-135b*, miR-148a, miR-194*, miR-21, miR-708, miR-222, miR-30a* and miR-323-3p; or
b. miR-122, miR-135b, miR-135b*, miR-136*, miR-186, miR-196b, miR-198, miR-203, miR-222, miR-23a, miR-34c-5p, miR-451, miR-490-3p, miR-492, miR-509-5p, miR-571, miR-614, miR-622 and miR-939.

81. The method according to claim 79, said method further comprising the step of extracting RNA from a sample collected from an individual.

82. The method according to claim 79, said method further comprising the step of correlating the miRNA expression level of at least one of said miRNAs to the expression level in a control sample.

83. The method according to claim 82, wherein said control sample is obtained from an individual having a normal pancreas and/or chronic pancreatitis.

84. The method according to claim 79, said method further comprising the step of calculating the difference in expression level of at least two miRNAs.

85. The method according to claim 79, wherein said pancreatic carcinoma is selected from the group consisting of pancreatic adenocarcinoma and ampullary adenocarcinoma.

86. The method according to claim 79, said method further comprising the step of determining if said individual has, or is at risk of developing, pancreatic carcinoma; such as pancreatic adenocarcinoma and/or ampullary adenocarcinoma.

87. The method according to claim 79, wherein said method comprises the step of obtaining prediction probabilities of between 0-1 for said sample in order to determine if said sample is classified as pancreatic carcinoma, normal pancreas or chronic pancreatitis.

88. The method according to claim 79, wherein said sample obtained from an individual is a tissue sample, such as a tissue sample from the pancreas.

89. The method according to claim 79, wherein miR-198 up-regulation is indicative of pancreatic carcinoma; miR-614 up-regulation is indicative of pancreatic carcinoma; and/or miR-122 down-regulation is indicative of pancreatic carcinoma.

90. The method according to claim 79, wherein the expression level of said at least one miRNA is determined by the microarray technique, by the quantitative polymerase chain reaction (QPCR) technique, by the northern blot technique, or by Nuclease protection assay.

91. The method according to claim 79, wherein said expression level of at least one miRNA is determined by contacting the sample with at least one probe or probe set for
a. miR-411 and miR-198; or
b. miR-614 and miR-122; or
c. miR-614 and miR-93*; or
d. miR-614; or
e. miR-122; or
f. miR-93*; or
g. miR-198.

92. The method according to claim 79, wherein the sample is extracted from an individual by fine-needle aspiration, by coarse-needle aspiration, by pancreatic surgery or by pancreatic biopsy.

93. The method according to claim 79, wherein said method is used in combination with at least one additional diagnostic method, said additional diagnostic method being selected from the group consisting of CT (X-ray computed tomography), MRI (magnetic resonance imaging), Scintillation counting, Blood sample analysis, Ultrasound imaging, Cytology, Histology, Assessment of risk factors, and the Asuragen test.

94. A miRNA classifier for characterizing a sample obtained from an individual, wherein said miRNA classifier comprises or consists of one or more miRNAs selected from the group consisting of:
i) miR-198, miR-34c-5p, miR-614, miR-492, miR-10a, miR-622, miR-196b, miR-210, miR-939, miR-649, miR-801, miR-135b*, miR-148a, miR-194*, miR-21, miR-708, miR-222, miR-30a* and miR-323-3p; or
ii) miR-122, miR-135b, miR-135b*, miR-136*, miR-186, miR-196b, miR-198, miR-203, miR-222, miR-23a, miR-34c-5p, miR-451, miR-490-3p, miR-492, miR-509-5p, miR-571, miR-614, miR-622 and miR-939,
wherein said miRNA classifier distinguishes the combined class of pancreatic carcinoma and ampullary adenocarcinoma from the combined class of normal pancreas and chronic pancreatitis, and wherein said distinction is given as a prediction probability for said sample of belonging to either class, said probability being a number falling in the range of from 0 to 1.

95. A device for measuring the expression level of at least one miRNA in a sample, wherein said device comprises or consists of at least one probe or probe set for at least one miRNA selected from the group consisting of:
i) miR-411 and miR-198, or
ii) miR-614 and miR-122, or
iii) miR-614 and miR-93*, or
iv) miR-198, miR-34c-5p, miR-614, miR-492, miR-10a, miR-622, miR-196b, miR-210, miR-939, miR-649, miR-801, miR-135b*, miR-148a, miR-194*, miR-21, miR-708, miR-222, miR-30a* and miR-323-3p, or
v) miR-122, miR-135b, miR-135b*, miR-136*, miR-186, miR-196b, miR-198, miR-203, miR-222, miR-23a, miR-34c-5p, miR-451, miR-490-3p, miR-492, miR-509-5p, miR-571, miR-614, miR-622 and miR-939,
wherein said device is used for characterizing a sample.

96. The device according to claim 95, wherein said device comprises or consists of:
a. at least one probe or probe set for miR-411 and at least one probe or probe set for miR-198; or
b. at least one probe or probe set for miR-614 and at least one probe or probe set for miR-122; or
c. at least one probe or probe set for miR-614 and at least one probe or probe set for miR-93*.

97. The device according to claim 95, wherein said device may be used for distinguishing between pancreatic carcinoma; comprising pancreatic carcinoma and ampullary adenocarcinoma; and normal pancreas and/or chronic pancreatitis.

98. The device according to claim 95, wherein said device is selected from the group consisting of a microarray chip; a microarray chip comprising DNA probes; a microarray chip comprising antisense miRNA probes; a QPCR Microfluidic Card; QPCR tubes; QPCR tubes in a strip; a QPCR plate; probes on a solid support; probes on at least one bead; and probes in liquid form in a tube.


SEQUENCE LISTING (31 sequences)

SEQ ID NO: 1 - RNA, 23 nt, Homo sapiens
  misc_feature (1)..(23): hsa-miR-10a
uacccuguagauccgaauuugug

SEQ ID NO: 2 - RNA, 22 nt, Homo sapiens
  misc_feature (1)..(22): hsa-miR-21
uagcuuaucagacugauguuga

SEQ ID NO: 3 - RNA, 22 nt, Homo sapiens
  misc_feature (1)..(22): hsa-miR-30a
cuuucagucggauguuugcagc

SEQ ID NO: 4 - RNA, 23 nt, Homo sapiens
  misc_feature (1)..(23): hsa-miR-34-5p
aggcaguguaguuagcugauugc

SEQ ID NO: 5 - RNA, 23 nt, Homo sapiens
  misc_feature (1)..(23): hsa-miR-93
caaagugcuguucgugcagguag

SEQ ID NO: 6 - RNA, 22 nt, Homo sapiens
  misc_feature (1)..(22): hsa-miR-122
uggagugugacaaugguguuug

SEQ ID NO: 7 - RNA, 22 nt, Homo sapiens
  misc_feature: hsa-miR-135b*
auguagggcuaaaagccauggg

SEQ ID NO: 8 - RNA, 22 nt, Homo sapiens
  misc_feature (1)..(22): hsa-miR-148a
ucagugcacuacagaacuuugu

SEQ ID NO: 9 - RNA, 22 nt, Homo sapiens
  misc_feature: hsa-miR-194*
ccaguggggcugcuguuaucug

SEQ ID NO: 10 - RNA, 22 nt, Homo sapiens
  misc_feature (1)..(22): hsa-miR-196b
uagguaguuuccuguuguuggg

SEQ ID NO: 11 - RNA, 22 nt, Homo sapiens
  misc_feature (1)..(22): hsa-miR-198
gguccagaggggagauagguuc

SEQ ID NO: 12 - RNA, 22 nt, Homo sapiens
  misc_feature (1)..(22): hsa-miR-210
cugugcgugugacagcggcuga

SEQ ID NO: 13 - RNA, 21 nt, Homo sapiens
  misc_feature (1)..(21): hsa-miR-222
agcuacaucuggcuacugggu

SEQ ID NO: 14 - RNA, 21 nt, Homo sapiens
  misc_feature (1)..(21): hsa-miR-323-3p
cacauuacacggucgaccucu

SEQ ID NO: 15 - RNA, 21 nt, Homo sapiens
  misc_feature (1)..(21): hsa-miR-411
uaguagaccguauagcguacg

SEQ ID NO: 16 - RNA, 23 nt, Homo sapiens
  misc_feature (1)..(23): hsa-miR-492
aggaccugcgggacaagauucuu

SEQ ID NO: 17 - RNA, 23 nt, Homo sapiens
  misc_feature (1)..(23): hsa-miR-614
gaacgccuguucuugccaggugg

SEQ ID NO: 18 - RNA, 21 nt, Homo sapiens
  misc_feature (1)..(21): hsa-miR-622
acagucugcugagguuggagc

SEQ ID NO: 19 - RNA, 22 nt, Homo sapiens
  misc_feature (1)..(22): hsa-miR-649
aaaccuguguuguucaagaguc

SEQ ID NO: 20 - RNA, 23 nt, Homo sapiens
  misc_feature (1)..(23): hsa-miR-708
aaggagcuuacaaucuagcuggg

SEQ ID NO: 21 - RNA, 24 nt, Homo sapiens
  misc_feature (1)..(24): hsa-miR-939
uggggagcugaggcucugggggug

SEQ ID NO: 22 - RNA, 22 nt, Homo sapiens
  misc_feature (1)..(22): hsa-miR-93*
acugcugagcuagcacuucccg

SEQ ID NO: 23 - RNA, 23 nt, Homo sapiens
  misc_feature (1)..(23): hsa-miR-135b
uauggcuuuucauuccuauguga

SEQ ID NO: 24 - RNA, 22 nt, Homo sapiens
  misc_feature (1)..(22): hsa-miR-136*
caucaucgucucaaaugagucu

SEQ ID NO: 25 - RNA, 22 nt, Homo sapiens
  misc_feature (1)..(22): hsa-miR-186
caaagaauucuccuuuugggcu

SEQ ID NO: 26 - RNA, 22 nt, Homo sapiens
  misc_feature (1)..(22): hsa-miR-203
gugaaauguuuaggaccacuag

SEQ ID NO: 27 - RNA, 21 nt, Homo sapiens
  misc_feature (1)..(21): hsa-miR-23a
aucacauugccagggauuucc

SEQ ID NO: 28 - RNA, 22 nt, Homo sapiens
  misc_feature (1)..(22): hsa-miR-451
aaaccguuaccauuacugaguu

SEQ ID NO: 29 - RNA, 22 nt, Homo sapiens
  misc_feature (1)..(22): hsa-miR-490-3p
caaccuggaggacuccaugcug

SEQ ID NO: 30 - RNA, 21 nt, Homo sapiens
  misc_feature (1)..(21): hsa-miR-509-5p
uacugcagacaguggcaauca

SEQ ID NO: 31 - RNA, 21 nt, Homo sapiens
  misc_feature (1)..(21): hsa-miR-571
ugaguuggccaucugagugag